(19) United States
(12) Patent Application Publication
(10) Pub. No.: US 2002/0064816 A1
(43) Pub. Date: May 30, 2002

(54) Moss genes from physcomitrella patens encoding proteins involved in the synthesis of carbohydrates

(76) Inventors: Jens Lerchl, Ladenburg (DE); Andreas Renz, Limburgerhof (DE); Thomas Ehrhardt, Speyer (DE); Andreas Reindl, Birkenheide (DE); Petra Cirpus, Mannheim (DE); Friedrich Bischoff, Mannheim (DE); Markus Frank, Ludwigshafen (DE); Annette Freund, Limburgerhof (DE); Elke Duwenig, Freiburg (DE); Ralf-Michael Schmidt, Kirrweiler (DE); Ralf Reski, Oberried (DE)

Correspondence Address: Herbert B. Keil, KEIL & WEINKAUF, 1101 Connecticut Ave., N.W., Washington, DC, 20036, US

(21) Appl. No.: 09/734,569

(22) Filed: Dec. 13, 2000

Related U.S. Application Data
(60) Provisional application No. 60/171,101, filed on Dec. 16, 1999.

(51) Int. Cl. C12P 21/02; C07H 21/04; C12N 9/24; C12N 5/04

(52) U.S. Cl. 435/69.1; 435/410; 536/23.2; 435/200

(57) ABSTRACT

Isolated nucleic acid molecules, designated CMRP nucleic acid molecules, which encode novel CMRPs from Physcomitrella patens are described. The invention also provides antisense nucleic acid molecules, recombinant expression vectors containing CMRP nucleic acid molecules, and host cells and organisms into which the expression vectors have been introduced. The invention still further provides isolated CMRPs, mutated CMRPs, fusion proteins, antigenic peptides and methods for the improvement of production of a desired compound from transformed cells based on genetic engineering of CMRP genes in this organism.

### BACKGROUND OF THE INVENTION

Certain products and by-products of naturally-occurring metabolic processes in cells have utility in a wide array of industries, including the food, feed, cosmetics, and pharmaceutical industries. These molecules, collectively termed ‘fine chemicals’, include carbohydrates, cofactors and enzymes.

Their production is most conveniently performed through the large-scale culture of microorganisms developed to produce and secrete large quantities of one or more desired molecules. One particularly useful organism for this purpose is Corynebacterium glutamicum, a gram positive, nonpathogenic bacterium.

Further particularly useful organisms for this purpose are Escherichia coli, Acetobacter xylinum and Chlorella. Through strain selection, a number of mutant strains of the respective microorganisms have been developed which produce an array of desirable compounds. However, selection of strains improved for the production of a particular molecule is a time-consuming and difficult process.

Alternatively the production of fine chemicals can be most conveniently performed via the large scale production of plants developed to produce one of aforementioned fine chemicals. Particularly well suited plants for this purpose are carbohydrate storing plants containing high amounts of carbohydrates like potato, maize, barley, wheat, rye, sugar cane, sugar beet, cotton, flax, poplar. But also other crop plants containing carbohydrates are well suited as mentioned in the detailed description of this invention. Through conventional breeding, a number of mutant plants have been developed which produce an array of desirable carbohydrates, cofactors and enzymes. However, selection of new plant cultivars improved for the production of a particular molecule is a time-consuming and difficult process or even impossible if the compound does not naturally occur in the respective plant as in the case of sugars like trehalose or raffinose.

### SUMMARY OF THE INVENTION

This invention provides novel nucleic acid molecules which may be used to modify carbohydrates, cofactors and enzymes in microorganims and plants, especially and most preferred to produce carbohydrates like starch, cell wall polysaccharids and soluble sugars. Microorganisms like Escherichia coli and Corynebacterium, fungi, green algae like Chlorella and plants are commonly used in industry for the large-scale production of a variety of fine chemicals.

Given the availability of cloning vectors for use in Corynebacterium glutamicum, such as those disclosed in Sinskey et al., U.S. Pat. No. 4,649,119, and techniques for genetic manipulation of C. glutamicum and the related Brevibacterium species (e.g., lactofermentum) (Yoshihama et al, J. Bacteriol. 162: 591-597 (1985); Katsumata et al., J. Bacteriol. 159: 306-311 (1984); and Santamaria et al., J. Gen. Microbiol. 130: 2237-2246 (1984)), the nucleic acid molecules of the invention may be utilized in the genetic engineering of this organism to make it a better or more efficient producer of one or more fine chemicals. This improved production or efficiency of production of a fine chemical may be due to a direct effect of manipulation of a gene of the invention, or it may be due to an indirect effect of such manipulation.

Given the availability of cloning vectors and techniques of genetic manipulation of bacteria such as Acetobacter xylinum described in Hall et al., Plasmid 28: 194-200 (1992) and references therein the nucleic acid molecules of the invention may be utilized in the genetic engineering of this organism to make it a better or more efficient producer or efficiency of production of a fine chemical may be due to a direct effect of manipulation of a gene of the invention, or it may be due to an indirect effect of such manipulation.

Given the availability of cloning vectors and techniques for genetic manipulation of algae such as Chlorella described in El-Sheekh, Biologia Plantarum 42: 209-216 (1999) as well as in Chow and Tung, Plant Cell Reports 18: 778-780 (1999) and references therein the nucleic acid molecules of the invention may be utilized in the genetic engineering of this organism to make it a better or more efficient producer of one or more fine chemicals. This improved production or efficiency of production of a fine chemical may be due to a direct effect of manipulation of a gene of the invention, or it may be due to an indirect effect of such manipulation.

Mosses as well as some algae and higher plants produce considerable amounts of starch, different cell wall polysaccharides and soluble sugars like sucrose, trehalose and raffinose. Therefore nucleic acid molecules originating from a moss like Physcomitrella patens are suitable to modify the carbohydrate production system in a host, especially in microorganisms and plants. Furthermore nucleic acids from the moss Physcomitrella patens can be used to identify those DNA sequences and enzymes in other species which are useful to modify the biosynthesis of starch, cell wall polysaccharides and soluble sugars. Nucleic acid molecules from Physcomitrella are of special interest for the functional analysis of genes since directed gene knock-out by homologous recombination is established for this moss as described in Hofmann et al., Molecular and General Genetics 261: 92-99 (1999) as well as in Girke et al., Plant Journal 15: 39-48 (1998).

The moss Physcomitrella patens represents one member of the mosses. It is related to other mosses such as Ceratodon purpureus which is capable to grow in the absense of light. Mosses like Ceratodon and Physcomitrella share a high degree of homology on the DNA sequence and polypeptide level allowing the use of heterologous screening of DNA molecules with probes evolving from other mosses or organisms, thus enabling the derivation of a consensus sequence suitable for heterologous screening or functional annotation and prediction of gene functions in third species. The ability to identify such functions can therefore have significant relevance, e.g. prediction of substrate specificity of enzymes. Further, these nucleic acid molecules may serve as reference points for the mapping of moss genomes, or of genomes of related organisms.

These novel nucleic acid molecules encode proteins, referred to herein as Carbohydrate Metabolism Related Proteins_(CMRPs). These CMRPs are capable of, for example, performing a function involved in the metabolism (e.g., the biosynthesis or degradation) of compounds necessary for carbohydrate biosynthesis or of influencing the structural properties of the carbohydrate, or of assisting in the transmembrane transport of one or more carbohydrate compounds or its metabolits either into or out of the cell. Given the availability of cloning vectors for use in plants and plant transformation, such as those published in and cited therein: Plant Molecular Biology and Biotechnology (CRC Press, Boca Raton, Fla.), chapter 6/7, S.71-119 (1993); F. F. White, Vectors for Gene Transfer in Higher Plants; in: Transgenic Plants, Vol. 1, Engineering and Utilization, eds.: Kung und R. Wu, Academic Press, 1993, 15-38; B. Jenes et al., Techniques for Gene Transfer, in: Transgenic Plants, Vol. 1, Engineering and Utilization, eds.: Kung und R. Wu, Academic Press (1993), 128-143; Potrykus, Annu. Rev. Plant Physiol. Plant Molec. Biol. 42 (1991), 205-225)) the nucleic acid molecules of the invention may be utilized in the genetic engineering of a wide variety of plants to make it a better or more efficient producer of one or more fine chemicals. This improved production or efficiency of production of a fine chemical may be due to a direct effect of manipulation of a gene of the invention, or it may be due to an indirect effect of such manipulation.

There are a number of mechanisms by which the alteration of an CMRP of the invention may directly affect the yield, production, and/or efficiency of production of a fine chemical from a carbohydrate storing plant due to such an altered protein. The nucleic acid and protein molecules of the invention may directly improve the production or efficiency of production of one or more desired fine chemicals from Corynebacterium glutamicum, other microorganisms and plants. Using recombinant genetic techniques well known in the art, one or more of the biosynthetic or degradative enzymes of the invention for amino acids, vitamins, cofactors, nutraceuticals, nucleotides or nucleosides may be manipulated such that its function is modulated. For example, a biosynthetic enzyme may be improved in efficiency, or its allosteric control region destroyed such that feedback inhibition of production of the compound is prevented. Similarly, a degradative enzyme may be deleted or modified by substitution, deletion, or addition such that its degradative activity is lessened for the desired compound without impairing the viability of the cell. In each case, the overall yield or rate of production of the desired fine chemical may be increased.

It is also possible that such alterations in the protein and nucleotide molecules of the invention may improve the production of other fine chemicals besides the amino acids, vitamins, cofactors, nutraceuticals, nucleotide and nucleosides through indirect mechanisms. Metabolism of any one compound is necessarily interwined with other biosynthetic and degradative pathways within the cell, and necessary cofactors, intermediates, or substrates in one pathway are likely supplied or limited by another such pathway. Therefore, by modulating the activity of one or more of the proteins of the invention, the production or efficiency of activity of another fine chemical biosynthetic or degradative pathway may be impacted. For example, amino acids serve as the structural units of all proteins, yet may be present intracellularly in levels which are limiting for protein synthesis; therefore, by increasing the efficiency of production or the yields of one or more amino acids within the cell, proteins, such as biosynthetic or degradative proteins, may be more readily synthesized. Likewise, an alteration in a metabolic pathway enzyme such that a particular side reaction becomes more or less favored may result in the over- or under-production of one or more compounds which are utilized as intermediates or substrates for the production of a desired fine chemical.

Those CMRPs involved in the transport of fine chemical molecules from the cell may be increased in number or activity such that greater quantities of these compounds are allocated to different plant cell compartments or the cell exterior space from which they are more readily recovered and partitioned into the biosynthetic flux or deposited. Similarly, those CMRPs involved in the import of nutrients necessary for the biosynthesis of one or more fine chemicals (e.g., sugar phosphates and nucleotide sugars) may be increased in number or activity such that these precursors, cofactors, or intermediate compounds are increased in concentration within the cell or within the storing compartments. Further, carbohydrates themselves are desirable fine chemicals; by optimizing the activity or increasing the number of one or more CMRPs of the invention which participate in the biosynthesis of these compounds, or by impairing the activity of one or more CMRPs which are involved in the degradation of these compounds, it may be possible to increase the yield, production, and/or efficiency of production of carbohydrates from plants or microorganisms. Further, the invention pertains to an isolated nucleic acid molecule which encodes an CMRP or an isolated CMRP polypepetide involved in assisting in transmembrane transport.

The mutagenesis of one or more CMRPs of the invention may also result in CMRPs having altered activities which indirectly impact the production of one or more desired fine chemicals from plants. For example, CMRPs of the invention involved in the export of waste products may be increased in number or activity such that the normal metabolic wastes of the cell (possibly increased in quantity due to the overproduction of the desired fine chemical) are efficiently exported before they are able to damage nucleotides and proteins within the cell (which would decrease the viability of the cell) or to interfere with fine chemical biosynthetic pathways (which would decrease the yield, production, or efficiency of production of the desired fine chemical). Further, the relatively large intracellular quantities of the desired fine chemical may in itself be toxic to the cell or may interfere with enzyme feedback mechanisms such as allosteric regulation, so by increasing the activity or number of transporters able to export this compound from the compartment, one may increase the viability of seed cells, in turn leading to a greater number of cells in the culture producing the desired fine chemical. The CMRPs of the invention may also be manipulated such that the relative amounts of different carbohydrates molecules are produced. This may have a profound effect on the carbohydrate composition and structure. E.g. a manipulation of starch metabolism results in a structurally altered starch as described in Lloyd et al., 1999, Planta 209: 230-238 and in Lloyd et al., 1999, Biochemical J. 338: 515-521. Also the manipulation of cell wall biosynthesis leads to altered carbohydrate composition as described in Keller et al., 1999, The Plant J. 19: 131-141, or in an altered structure and physical property of the cell wall as described in Taylor et al., 1999, Plant Cell 11: 769-779. Changes in starch structure can influence its physical properties and also its digestibility. Changes in the cell wall structure can impact the integrity of the cell as well as the stability of plant organs and whole plants. This can in turn influence other characteristics like tolerance towards abiotic and biotic stress conditions.

The invention provides novel nucleic acid molecules which encode proteins, referred to herein as CMRPs, which are capable of, for example, participating in the metabolism of compounds necessary for the construction of carbohydrates. Nucleic acid molecules encoding an CMRP are referred to herein as CMRP nucleic acid molecules. In a preferred embodiment, the CMRP participates in the metabolism of compounds necessary for the construction of carbohydrates in plants. Examples of such proteins include those encoded by the genes set forth in Table 1.

As biotic and abiotic stress tolerance is a general trait wished to be inherited into a wide variety of plants like maize, wheat, rye, oat, triticale, rice, barley, soybean, peanut, cotton, flax, rapeseed and canola, manihot, pepper, sunflower and tagetes, solanaceaous plants like potato, tobacco, eggplant, and tomato, Vicia species, pea, alfalfa, bushy plants (coffee, cacao, tea), Salix species, trees (poplar, elm) and perennial grasses and forage crops, these crops plants are also preferred target plants for a genetic engineering as one futher embodiment of the present invention.

Accordingly, one aspect of the invention pertains to isolated nucleic acid molecules (e.g. cDNAs) comprising a nucleotide sequence encoding an CMRP or biologically active portions thereof, as well as nucleic acid fragments suitable as primers or hybridization probes for the detection or amplification of CMRP-encoding nucleic acid (e.g., DNA or mRNA). In another embodiment, the isolated nucleic acid molecule is at least 15 nucleotides in length and hybridizes under stringent conditions to a nucleic acid molecule comprising a nucleotide sequence of Appendix A (SEQ ID NO:1 to SEQ ID NO:177, odd integers). Preferably, the isolated nucleic acid molecule corresponds to a naturally-occurring nucleic acid molecule. More preferably, the isolated nucleic acid encodes a naturally-occurring Physcomitrella patens CMRP, or a biologically active portion thereof. In particularly preferred embodiments, the isolated nucleic acid molecule comprises one of the nucleotide sequences set forth in Appendix A (SEQ ID NO:1 to SEQ ID NO:177, odd integers) or the coding region or a complement thereof of one of these nucleotide sequences. In other particularly preferred embodiments, the isolated nucleic acid molecule of the invention comprises a nucleotide sequence which hybridizes to or is at least about 50%, preferably at least about 60%, more preferably at least about 70%, 80% or 90%, and even more preferably at least about 95%, 96%, 97%, 98%, 99% or more homologous to a nucleotide sequence set forth in Appendix A (SEQ ID NO:1 to SEQ ID NO:177, odd integers), or a portion thereof In other preferred embodiments, the isolated nucleic acid molecule encodes one of the amino acid sequences set forth in Appendix B (SEQ ID NO:2 to SEQ ID NO:178, even integers). The preferred CMRPs of the present invention also preferably possess at least one of the CMRP activities described herein.

In another embodiment, the isolated nucleic acid molecule encodes a protein or portion thereof wherein the protein or portion thereof includes an amino acid sequence which is sufficiently homologous to an amino acid sequence of Appendix B (SEQ ID NO:2 to SEQ ID NO:178, even integers), e.g., sufficiently homologous to an amino acid sequence of Appendix B (SEQ ID NO:2 to SEQ ID NO:178, even integers) such that the protein or portion thereof maintains an CMRP activity. Preferably, the protein or portion thereof encoded by the nucleic acid molecule maintains the ability to participate in the metabolism of compounds necessary for the construction of carbohydrates of plants. In one embodiment, the protein encoded by the nucleic acid molecule is at least about 50%, preferably at least about 60%, and more preferably at least about 70%, 80%, or 90% and most preferably at least about 95%, 96%, 97%, 98%, or 99% or more homologous to an amino acid sequence of Appendix B (SEQ ID NO:2 to SEQ ID NO:178, even integers) (e.g., an entire amino acid sequence selected from those sequences set forth in Appendix B). In another preferred embodiment, the protein is a full length Physcomitrella patens protein which is substantially homologous to an entire amino acid sequence of Appendix B (SEQ ID NO:2 to SEQ ID NO:178, even integers) (encoded by an open reading frame shown in Appendix A (SEQ ID NO:1 to SEQ ID NO:177, odd integers)).

In another preferred embodiment, the isolated nucleic acid molecule is derived from Physcomitrella patens and encodes a protein (e.g., an CMRP fusion protein) which includes a biologically active domain which is at least about 50% or more homologous to one of the amino acid sequences of Appendix B (SEQ ID NO:2 to SEQ ID NO:178, even integers) and is able to participate in the metabolism of compounds necessary for the construction of carbohydrates, or has one or more of the activities set forth in Table 1, and which also includes heterologous nucleic acid sequences encoding a heterologous polypeptide or regulatory regions.

Another aspect of the invention pertains to a CMRP whose amino acid sequence can be modulated with the help of art-known computer simulation programms resulting in an polypeptide with e.g. improved activity or altered regulation (molecular modelling). On the basis of this artificially generated polypeptide sequences, a corresponding nucleic acid molecule coding for such a modulated polypeptide can be synthesized in-vitro using the specific codon-usage of the desired host cell, e.g. of microorganisms, mosses, algae, ciliates, fungi or plants. In a preferred embodiment, even these artificial nucleic acid molecules coding for improved CMRPs are within the scope of this invention.

Another aspect of the invention pertains to vectors, e.g., recombinant expression vectors, containing the nucleic acid molecules of the invention, and host cells into which such vectors have been introduced, especially microorganims, plant cells, plant tissue, organs or whole plants. In one embodiment, such a host cell is a cell capable of storing fine chemical compounds in order to isolate the desired compound from harvested material. The compound or the CMRP can then be isolated from the medium or the host cell, which in plants are cells containing and storing fine chemical compounds, most preferably cells of storage tissues like tubers, roots or seeds. Preferred are also cells like phloem fibres and cotton fibres.

Yet another aspect of the invention pertains to a genetically altered Physcomitrella patens plant in which an CMRP gene has been introduced or altered. In one embodiment, the genome of the Physcomitrella patens plant has been altered by introduction of a nucleic acid molecule of the invention encoding wild-type or mutated CMRP sequence as a transgene. In another embodiment, an endogenous CMRP gene within the genome of the Physcomitrella patens plant has been altered, e.g., functionally disrupted, by homologous recombination with an altered CMRP gene. In a preferred embodiment, the plant organism belongs to the genus Physcomitrella or Ceratodon, with Physcomitrella being particularly preferred. In a preferred embodiment, the Physcomitrella patens plant is also utilized for the production of a desired compound, such as carbohydrates, with starch, cell wall carbohydrates, sucrose, trehalose and raffinose being particularly preferred.

Hence in another preferred embodiment, the moss Physcomitrella patens can be used to show the function of a moss gene using homologous recombination based on the nucleic acids described in this invention.

Still another aspect of the invention pertains to an isolated CMRP or a portion, e.g., a biologically active portion, thereof. In a preferred embodiment, the isolated CMRP or portion thereof can participate in the metabolism of compounds necessary for the construction of carbohydrates in a microorganism or a plant cell, or in the transport of sugar metabolites across its membranes. In another preferred embodiment, the isolated CMRP or portion thereof is sufficiently homologous to an amino acid sequence of Appendix B (SEQ ID NO:2 to SEQ ID NO:178, even integers) such that the protein or portion thereof maintains the ability to participate in the metabolism of compounds necessary for the construction of carbohydrates in microorganisms or plant cells.

The invention also provides an isolated preparation of an CMRP. In preferred embodiments, the CMRP comprises an amino acid sequence of Appendix B (SEQ ID NO:2 to SEQ ID NO:178, even integers). In another preferred embodiment, the invention pertains to an isolated full length protein which is substantially homologous to an entire amino acid sequence of Appendix B (SEQ ID NO:2 to SEQ ID NO:178, even integers) (encoded by an open reading frame set forth in Appendix A (SEQ ID NO:1 to SEQ ID NO:177, odd integers)). In yet another embodiment, the protein is at least about 50%, preferably at least about 60%, and more preferably at least about 70%, 80%, or 90%, and most preferably at least about 95%, 96%, 97%, 98%, or 99% or more homologous to an entire amino acid sequence of Appendix B (SEQ ID NO:2 to SEQ ID NO:178, even integers). In other embodiments, the isolated CMRP comprises an amino acid sequence which is at least about 50% or more homologous to one of the amino acid sequences of Appendix B (SEQ ID NO:2 to SEQ ID NO:178, even integers) and is able to participate in the metabolism of compounds necessary for the construction of carbohydrates in a microorganism or a plant cell, or has one or more of the activities set forth in Table 1.

Alternatively, the isolated CMRP can comprise an amino acid sequence which is encoded by a nucleotide sequence which hybridizes, e.g., hybridizes under stringent conditions, or is at least about 50%, preferably at least about 60%, more preferably at least about 70%, 80%, or 90%, and even more preferably at least about 95%, 96%, 97%, 98,%, or 99% or more homologous, to a nucleotide sequence of Appendix B (SEQ ID NO:2 to SEQ ID NO:178, even integers). It is also preferred that the preferred forms of CMRPs also have one or more of the CMRP activities described herein.

The CMRP polypeptide, or a biologically active portion thereof, can be operatively linked to a non-CMRP polypeptide to form a fusion protein. In preferred embodiments, this fusion protein has an activity which differs from that of the CMRP alone. In other preferred embodiments, this fusion protein participates in the metabolism of compounds necessary for the synthesis of carbohydrates, cofactors and enzymes and structural proteins in microorganisms or plants, or in the transport of sugar metabolites across the membranes of plants. In particularly preferred embodiments, integration of this fusion protein into a host cell modulates production of a desired compound from the cell. Further the instant invention pertains to an antibody specifically binding to an CMRP polypeptide mentioned before or to a portion thereof

Another aspect of the invention pertains to a test kit comprising a nucleic acid molecule encoding a CMRP protein, a portion and/or a complement of this nucleid acid molecule used as probe or primer for identifying and/or cloning further nucleic acid molecules involved in the synthesis of amino acids, vitamis, cofactors, nucloetides and/or nucleosides or assisting in transmembrane transport in other cell types or organisms. In another embodiment the test kit comprises a CMRP-antibody for identifying and/or purifying further CMRP molecules or fragments thereof in other cell types or organisms.

Another aspect of the invention pertains to a method for producing a fine chemical. This method involves either the culturing of a suitable microorganism or culturing plant cells tissues, organs or whole plants containing a vector directing the expression of an CMRP nucleic acid molecule of the invention, such that a fine chemical is produced. In a preferred embodiment, this method further includes the step of obtaining a cell containing such a vector, in which a cell is transformed with a vector directing the expression of an CMRP nucleic acid. In another preferred embodiment, this method further includes the step of recovering the fine chemical from the culture. In a particularly preferred embodiment, the cell is from the genus Escherichia, Corynebacterium, fungi, from carbohydrate storing plants or from fibre plants.

Another aspect of the invention pertains to a method for producing a fine chemical which involves the culturing of a suitable host cell whose genomic DNA has been altered by the inclusion of an CMRP nucleic acid molecule of the invention. Further, the invention pertains to a method for producing a fine chemical which involves the culturing of a suitable host cell whose membrane has been altered by the inclusion of an CMRP of the invention.

Another aspect of the invention pertains to methods for modulating production of a molecule from a microorganism. Such methods include contacting the cell with an agent which modulates CMRP activity or CMRP nucleic acid expression such that a cell associated activity is altered relative to this same activity in the absence of the agent. In a preferred embodiment, the cell is modulated for one or more metabolic pathways for carbohydrates, cofactors, enzymes or structural proteins or is modulated for the transport of sugar metabolites across such membranes, such that the yields or rate of production of a desired fine chemical by this microorganism is improved. The agent which modulates CMRP activity can be an agent which stimulates CMRP activity or CMRP nucleic acid expression. Examples of agents which stimulate CMRP activity or CMRP nucleic acid expression include small molecules, active CMRPs, and nucleic acids encoding CMRPs that have been introduced into the cell. Examples of agents which inhibit CMRP activity or expression include small molecules and antisense CMRP nucleic acid molecules.

Another aspect of the invention pertains to methods for modulating yields of a desired compound from a cell, involving the introduction of a wild-type or mutant CMRP gene into a cell, either maintained on a separate plasmid or integrated into the genome of the host cell. If integrated into the genome, such integration can be random, or it can take place by recombination such that the native gene is replaced by the introduced copy, causing the production of the desired compound from the cell to be modulated or by using a gene in trans such as the gene is functionally linked to a functional expression unit containing at least a sequence facilitating the expression of a gene and a sequence facilitating the polyadenylation of a functionally transcribed gene.

In a preferred embodiment, said yields are modified. In another preferred embodiment, said desired chemical is increased while unwanted disturbing compounds can be decreased. In a particularly preferred embodiment, said desired fine chemical is carbohydrate, cofactor, enzyme or structural protein. In especially preferred embodiments, said chemicals are starch, cell wall polysaccharides and soluble sugars.

Another aspect of the invention pertains to the fine chemicals produced by a method described before and the use of the fine chemical or a polypeptide of the invention for the production of another fine chemical.

### DETAILED DESCRIPTION OF THE INVENTION

The present invention provides CMRP nucleic acid and protein molecules which are involved in the metabolism of carbohydrates, cofactors, enzymes and structural proteins in the moss Physcomitrella patens. The molecules of the invention may be utilized in the modulation of production of fine chemicals from microorganisms, such as Corynebacterium, fungi, algae and plants like maize, wheat, rye, oat, triticale, rice, barley, soybean, sugar cane, sugar beet, cotton, flax, poplar, Brassica species like rapeseed, canola and turnip rape, pepper, sunflower and tagetes, solanaceaous plants like potato, tobacco, eggplant, and tomato, Vicia species, pea, manihot, alfalfa, bushy plants (coffee, cacao, tea), Salix species, trees (poplar, elm) and perennial grasses and forage crops either directly (e.g., where overexpression or optimization of a carbohydrate biosynthesis protein has a direct impact on the yield, production, and/or efficiency of production of the carbohydrate from modified organisms), or may have an indirect impact which nonetheless results in an increase of yield, production, and/or efficiency of production of the desired compound or decrease of undesired compounds (e.g., where modulation of the metabolism of carbohydrates, cofactors, enzymes or structural proteins results in alterations in the yield, production, and/or efficiency of production or the composition of desired compounds within the cells, which in turn may impact the production of one or more fine chemicals). Aspects of the invention are further explicated below.

Fine Chemicals

The term ‘fine chemical’ is art-recognized and includes molecules produced by an organism which have applications in various industries, such as, but not limited to, pharmaceutical, agriculture, and cosmetics industries. Such compounds include carbohydrates, cofactors, enzymes, structural proteins (as described e.g. in Kuninaka, A. (1996) and nucleotides and related compounds, p. 561-612, in Biotechnology vol. 6, Rehim et al., eds. VCH: Weinheim, and references contained therein), carbohydrates (e.g., starch, amylopectine, amylose, cellulose, hemicelluloses, pectins, sucrose, trehalose, raffinose) Encyclopedia of Industrial Chemistry, vol. A27; Chemicals by Fermentation, Noyes Data Corporation, ISBN: 0818805086 and references therein. The metabolism and uses of certain of these fine chemicals are further explicated below.

Carbohydrates

Carbohydrates can be divided into polymeric carbohydrates like starch, fructans and cell wall polysaccharides (cellulose, hemicelluloses and pectins) on the one hand and soluble mono- and oligosaccharides on the other hand.

Polysaccharides like starch serve as an energy reserve, either as transitory starch that is built up within the leaves during the day and is degraded during the night, or as reserve starch, that is deposited in storage organs like tubers, roots and seeds. More than 20 million tons of starch are isolated each year to serve for a wide range of industrial applications, such as the coating of textiles and paper, or as a thickening of gelling agent in the food industry (see Lillford, P. J. and Morrison, A, in ‘Starch—Structure and Functionality’, p. 1-8, edited by Frazier, P. J., Donald, A. M., Richmond, Cambridge: The Royal Society of Chemistry, 1997). Starch is constituted of 20-30% of the essentially linear polymer amylose in which the glucose is polymerized via alpha-1,4-glycosidic linkages. 70-80% of the starch is accounted for by amylopectin, which has a higher molecular weight than amylose and is much more frequently branched (via alpha-1,6-glycosidic linkages). These branchpoints are arranged in clusters, allowing the formation of alpha-helices and resulting in a semi-crystalline amylopectin phase (reviewed in Smith, A. M., Denyer, K., Martin, C. (1997) Annu. Rev. Plant Physiol. Plant Mol. Biol. 48: 67-87). Furthermore the glucose moieties of amylopectin can be phosphorylated at the C-3 or C-6 position, with an especially high phosphate content in the starch of tuberous plant species like potato (see Jane, J., Kasemsuwan, T., Chen, J. F., Juliano, B. O. (1996) Cereal Foods World 41: 827-832).

Cell wall polysaccharides fulfill structural, protective and growth regulating functions within the lifecycle of a plant cell and the whole plant. The cell wall contains different classes of polysaccharides. Cellulose, which consists of beta-1,4-linked glucose units, forms semi-crystalline microfibrills that imparts mechanical strength to the cell and represents the world's most abundant biopolymer, being an important raw material for the fibre and paper industry. The cellulose microfibrills are embedded in a matrix of hemicellulose and pectic polysaccharides. Hemicelluloses have a carbohydrate backbone structurally similar to celluose and are cross-linked to cellulose microfibrills via strong hydrogen-bond interactions. Xyloglucan is the predominant hemicellulose in the primary cell wall of most dicotyledonous plants. It consists of linear beta-1,4-glucan chains that contain xylosyl units. Hemicelluloses of monocotyledonous plants contain little xyloglucans and pectins, but high amounts of xylans and mixed-linked glucans (short blocks of beta-1,4-inked glucose molecules connected via beta-1,3-glycosidic bonds). Pectins are highly negatively charged polysaccharides, mainly consisting of polygalacturonic acid and rhamnogalacturonan I. They appear to form a three-dimensional network that is interwined with the cellulose-xyloglucan network. In addition to polysaccharides, plant cell walls contain structural proteins like hydroxyproline-rich glycoproteins (e.g. extensins) and enzymes (e.g. expansins and various glucan hydrolases) that are essential for cell expansion and fruit ripening by loosening the cellulose-hemicellulose connections. A model of the plant cell wall structure is reviewed in Carpita, N. C. and Gibeaut, D. M. (1993) The Plant J. 3: 1-30 and in Rose, J. K. C. and Bennett, A. B. (1999) Trends in Plant Science 4: 176-183.

Soluble mono- and oligosaccharides contain a wide variety of sugars that serve either as metabolites or as transport and storage forms of carbohydrates. Many monosaccharides are metabolites of the primary metabolism that are further converted to polysaccharides (such as glucose, fructose, fucose, ribose, xylose, xyluluse, galactose etc.) or other fine chemicals like amino acids by the formation of sugar phosphates and nucleotide sugars. Regulation and interaction of different pathways of the primary metabolism is reviewed in Siedow, J. N. and Stitt, M. (1998) Current Opinion in Plant Biology 1: 197-200. There are several reviews to date summarizing the efforts to modify carbohydrate metabolism and partitioning via soluble sugars and sugar phosphates (see including references therein: Sonnewald et al. 1994, Plant, Cell and Environment, 17:1-10; Frommer & Sonnewald 1995, J. Experim. Botany, Vol 46, 287:587-607). The disaccharide sucrose is the major transport form of carbohydrates in plants. In some species like sugar cane and sugar beet, however, sucrose is also the storage form. The cleavage of sucrose is crucial for development, growth and carbon partitioning in plants and is thus highly regulated (reviewed in Sturm, A. and Tang, G. -Q. (1999) Trends in Plant Science 4: 401-407). In some members of the Cucurbitaceae the trisaccharide raffinose serves as an alternative transport form of carbohydrates. Moreover, raffinose plays an important role in desiccation tolerance as described in Brenac, P., Smith, M. E., Obendorf, R. L. (1997) Planta 203: 222-228. Raffinose has many applications, e.g. in organ transplantation and preservation (reviewed in Southard, J. H. and Belzer, F. O. (1995) Annual Review of Medicine 46: 235-247). The disaccharide trehalose is composed of two glucose moieties. Its role in plants is not fully clarified, however, it is discussed to be a regulatory component in the control of glycolytic flux and in a variety of stress survival strategies (see Goddijn O. J. M. and van Dun, K. (1999) Trends in Plants Science 4: 315-319).

Starch

Starch metabolism is mainly localized in the plastids of plant cells. A prerequisite for efficient starch metabolism is therefore the transport of sugar phosphates from the cytosol into the plastids (reviewed in Pozueta-Romero, J. Perata, P. and Akazawa, T. (1999) Critical Reviews in Plant Sciences 18: 489-525). In photosynthetic tissues the phosphateltriose phosphate translocator plays a crucial role in the partitioning of photosynthetic assimilates (Flügge, U. I. (1999) Annual Review Plant Physiol. Plant Mol. Biol. 50: 27-45). Plastids of heterotrophic tissues contain ATP/ADP translocators (e.g. Neuhaus, H. E., Henrichs, G. and Scheibe, R. (1993) Plant Physiol. 101: 573-578) and are able to import glucose-1-phosphate and glucose-6-phosphate (e.g. Neuhaus, H. E., Batz, O., Thom, E. and Scheibe, R. (1993) Biochem. S. 196: 395-401). ADP-glucose is also imported into amyloplasts via a specific translocator (Shannon, J. C., Pien, F. -M., Cao, H. and Liu, K. -C. (1998) Plant Physiol. 117: 1235-1252). The initial step in starch biosynthesis within the plastids is the conversion of glucose-1-phosphate to ADP-glucose by ADP-glucose-pyrophosphorylase. ADP-glucose then serves as a substrate for starch synthases. These catalyze the chain elongation by transferring the glucose moiety from ADP-glucose to alpha-1,4-glucans. At least four different starch synthases are known. The different isoforms contribute in various degree to the incorporation of glucose into starch. One isoform, the granule bound starch synthase, is responsible for the synthesis of amylose. Starch from waxy mutants lacking granule bound starch synthase (known from maize, rice and potato) are essentially amylose free (see e.g. Hovenkamp-Hermelink et al. (1987) Theor. Appl. Genet. 75: 217-221). In the mutants dull1 in maize and rugosus5 in pea, other starch synthases are affected, leading to reduced starch yield and altered amylopectin structure (see Gao, M. et al. (1998) Plant Cell 10: 399-412 and Craig, J. et al. (1998) Plant Cell 10: 413-426). At least two branching enzyme isoforms are responsible for the introduction of branchpoints, i.e. for the production of amylopectin (see Martin, C. and Smith, A. M. (1995) Plant Cell 7:971-985 and literature cited therein). Debranching enzymes, originally known to be involved in starch breakdown (see below) are also involved in starch biosynthesis by ‘trimming’ highly branched glucans to amylopectin. This was shown by the analysis of sugary-1 mutants of rice that accumulate highly branched glucans and are reduced in the activity of both debranching enzymes (see Nakamura, Y. et al. (1999) Plant Physiol. 121: 399-409 and Smith, A. M. (1999) Current Opinion in Plant Biology 2: 223-229).

The mechanism and the function of starch phosphorylation is not yet fully understood. In potato, however, a granule bound protein was shown to be involved in starch phosphorylation (see Lorberth, R., Ritte, G., Willmitzer, L. and Kossmann, J. (1998) Nature Biotechnol. 16: 473-477). Antisense plants with strongly reduced expression levels of the corresponding gene produced essentially unphosphorylated starch and showed a so-called ‘starch excess phenotype’, i.e. the unphosphorylated starch was not amenable to the starch degrading enzyme system of the plant. Starch biosynthesis is reviewed in Smith, A. M. (1999) Current Opinion in Plant Biology 2: 223-229 and in Heyer, A. G., Lloyd, J. R., Kossmann, J. (1999) Current Opinion in Biotechnology 10: 169-174.

The hydrolytic starch degrading enzymes include alpha- and beta-amylases that hydrolyse alpha-1,4-linkages of starch. Several amylase-isoenzymes are present in plants, some of them being localized in the plastid, some outside of it. The function of extraplastidial isoenzymes is still unclear. Debranching enzymes hydrolyse the alpha-1,6-linkages of amylopectin. There are two classes of debranching enzymes in plants: isoamylase and pullulanase (r-enzyme), differing with respect to their substrate specificties and protein heterogeneity. The so-called disproportionating enzyme (d-enzyme) transfers short side chains within the starch molecule, thus producing longer glucan chains, that can be hydrolysed by amylases and debranching enzymes (Kakefuda, G. and Duke, S. H. (1989) Plant Physiol. 91: 136-143). Maltooligosaccharides and maltose are hydrolysed by alpha-gucosidase (maltase), producing glucose which is again phosphorylated by hexokinase. The resuling glucose-6-phosphate is part of the hexose phosphate pool that is part of various metabolic pathways. In the phosphorolytic starch degradation inorganic phosphate, instead of water, serves as a glucosyl-acceptor. In a reversible reaction, starch phosphorylase cleaves glucose from the non-reducing end of a glucan chain and transfers it to inorganic phosphate, thus producing glucose-1-phosphate. Several isoforms of starch phosphorylase are described in Duwenig, E., Steup, M., Willmitzer, L., Kossmann, J. (1999) Plant J. 12: 323-333 with the cytosolic form being involved in potato tuber sprouting and flower formation.

The biosynthesis of starch is a highly regulated pathway, e.g. ADP-glucose-pyrophosphorylase is an allosteric enzyme effected by various metabolites. Moreover, indirect evidence of protein-protein-interaction of starch biosynthetic enzymes exist, as the parallel antisense-inhibition of two or more starch biosynthetic enzymes often leads to dramatic effects on starch structure (see e.g. Lloyd, J. R., Landschuitze, V., Kossmann, J. (1999) Biochem. J. 338: 515-521).

The heterologous expression of starch biosynthetic enzymes may not only alter the amount of starch produced by a transformed organism, but may have a significant effect on the starch quality (e.g. amylose content, chains length distribution, physical properties, phosphate content, digestability). Moreover, a functional gene analysis (e.g. directed gene knock-out in the moss Physcomitrella patens) will give important informations about the function of various isoenzymes and thus far poorly characterized enzymes of starch metabolism.

Cell wall carbohydrates

The biosynthesis of semi-cristalline cellulose microfibrills starts with the enzyme sucrose synthase that cleaves sucrose into fructose and UDP-glucose. The latter is the substrate for the plasmalemma bound multienzyme complex of cellulose synthase which forms the so-called rosette complexes. The catalytic subunit of cellulose synthase, CelA, a transmembrane protein, was cloned from several plant species including cotton, poplar and Arabidopsis. Several CelA isoforms with tissue- and development specific expression are described (see Delmer, D. P. (1999) Annual Review Plant Physiol. Plant Mol. Biol. 50: 245-276). Although CelA belongs to a multigene family, the disruption of a single isoform (rsw1) results in the disassembly of rosette complexes, a dramatic reduction of the cellulose content and the accumulation of non-crystalline beta-1,4-glucans in the cell wall (Arioli, T. et al. (1998) Science 279: 717-720). Arabidopsis irx3 mutants show a severe deficiency in secondary cell wall cellulose deposition which leads to collapsed xylem cells. A close interaction between a membrane associated sucrose synthase and cellulose synthase was shown by Nakai, T. et al. (1999) Proc. Natl. Acad. Sci. U.S.A. 96: 14-18 who showed that cellulose biosynthesis in Acetobacter xylinurm is enhanced by the overexpression of sucrose synthase. Moreover several lines of evidence exist for a protein-protein interaction between sucrose synthase and cellulose synthase (Delmer, D. P. and Amor, Y. (1995) Plant Cell 7: 987-1000). All components of the cellulose synthase complex are characterized in Acetobacter and Agrobacterium (see e.g. Mattysse, A. G., White, S. and Lightfoot, R. (1995) J. Bacteriol. 177: 1069-1075). In plants, a membrane associated endo-beta-D-glucanase is discussed to be part of the cellulose synthase complex (Brummell, D. A., Catala, C., Lashbrook C. C., Bennett, A. B. (1997) Proc. Natl. Acad. Sci. U.S.A. 94: 4794-4799).

The biosynthesis of non-cellulosic cell wall polysaccharides can be devided into four stages: (i) Formation of activated monosaccharides via nucleotide sugar interconversion pathways. (ii) Translocation of these precursors from the cytosol into the lumen of the endomembrane system. (iii) synthesis of polysaccharides from the nucleotide sugars. (iv) Modification of the polysaccharides in the apoplastic space.

The enzymes involved in the nucleotide sugar interconversion pathway are described in detail by Feingold, D. S. and Barber, G. A. (1990) Nucleotide sugars. In: Dey, P. M. (ed.) Methods in Plant Biochemistry, vol. 2. Carbohydrates. Academic Press, London, pp. 39-78. Several plant genes involved in the nucleotide sugar interconversion have been described, e.g. UDP-D-glucose dehydrogenase (Tenhaken, R. and Thulke, O. (1996) Plant Physiol. 112: 1127-1134), UDP-D-glucose 4-epimerase (Dörrmann, P. and Benning, C. (1996) Arch. Biochem. Biophys. 327: 27-34), GDP-D-mannose 4,6-dehydratase (Bonin, C. P. et al. (1997) Proc. Natl. Acad. Sci. U.S.A. 94: 2085-2090) and GDP-D-mannose pyrophosphorylase (Keller, R., Springer, F., Renz, A. and Kossmann, J. (1999) Plant J. 19: 131-141 and Conklin, P. L. et al. (1999) Proc. Natl. Acad. Sci. U.S.A. 96: 4198-4203), with GDP-D-mannose 4,6-dehydratase and GDP-D-mannose pyrophosphorylase corresponding to known mutations in Arabidopsis (mur1 and vtc1, respectively). Besides murk, ten more, independent Arabidopsis mutants are known showing an altered cell wall monosaccharide composition (Reiter, W. D., Chapple, C. and Somerville, C. R. (1997) Plant J. 12: 335-345). Manipulation of genes involved in nucleotide sugar interconversions is of considerable interest, because they act at an early step in cell wall synthesis, and may therefore serve as important regulators. Moreover, nucleotide sugars are not only involved in cell wall biosynthesis, but also in pathways like protein glycosylation and vitamin c biosynthesis. E.g. Arabidopsis mur1 mutants do not only have reduced fucose contents in cell wall polysaccharides, but also show reduced fucose levels in N-linked glycans of glycoproteins (Rayon, C. et al. (1999) Plant Physiol. 119: 725-734). Transgenic potato plants with reduced GDP-D-mannose pyrophosphorylase activity do not only show reduced cell wall mannose contents, but also significantly reduced ascorbate levels, leading to a severe damage of the aerial part of the plants (Keller, R. et al. (1999) Plant J. 19: 131-141). These data imply, that genetic manipulation of the nucleotide interconversion pathway is a promising target in plant biotechnology.

The translocation of nucleotide sugar into the lumen of the endomembrane system is not well understood in plants. In biochemical studies it was shown that e.g. UDP-glucose and UDP-galacturonic acid are transported into the Golgi apparatus (Nunoz, P., Norambuena, L. and Orellana, A. (1996) Plant Physiol. 112: 1585-1594 and Orellana, A., Mohnen, D. (1999) Analyt. Biochem. 272: 224-231, respectively). Several nucleotide sugar transporters are known from animals and yeast (reviewed in Kawakita, M. et al. (1998) J. Biochem. 123: 777-785). Thus, it should be possible to isolate plant homologs in the near future.

Non-cellulosic polysaccharides are synthesized from nucleotide sugar precursors by glycosyltransferases that are localized in the Golgi apparatus (for xylosyl- and glucuronyltransferases see e.g. Baydoun, E. A. -H. and Brett, C. T. (1997) J. Exp. Bot. 48: 1209-1214). The so-called cellulose synthase-like (Csl) genes, that form a multigene family of about 17 members, are discussed to code for glycosyltransferases, e.g. xyloglucan synthases (Cutler, S. and Somerville, C. (1997) Current Biology 7: R108-R111). Csl genes could be characterized by functional genomic approaches like gene disruption and heterologous gene expression. The correct targeting of a foreign glycosyltransferase gene into the plant golgi apparatus was shown by Wee, E. G. T., Sherrier, D. J., Prime, T. A. and Dupree, P. (1998) Plant Cell 10: 1759-1768.

Different glycosyltransferases involved in pectin biosynthesis are biochemically characterised, e.g. galacturonosyltransferases (see Doong, R. L. et al. (1998) Plant J. 13: 363-374). Others are reviewed in Gibeaut, D. M. and Carpita, N. C. (1994) FASEB J. 8: 904-915. Plant enzymes involved in pectin degradation, e.g. pectin methylesterase, polygalacturonases and pectate lyases are biochemically and genetically characterised. Pectin degradation plays an important role in fruit ripening (reviewed in Hadfield, K. A. and Bennett, A. B. (1997) Cell Death and Differentiation 4: 662-670) and cell adhesion (see e.g. Rhee, S. Y. and Somerville, C. R. (1998) Plant J. 15: 79-88). The manipulation of pectin degradation was applied for the production of plants with delayed senescence or modified pectins (reviewed in Tucker, G. A., Simons, H. and Errington, N. (1999) Biotech. Genet. Engin. Rev. 16: 293-308).

The modification of cell wall polysaccharides in the apoplastic space involves a variety of enzymes as well as structural proteins. Xyloglucan endotransglycosylases have been cloned from various plants and are proposed to catalyse the intramolecular cleavage of xyloglucans and transfer the newly generated, potentially reducing end, to another xyloglucan chain. They form a multigene family and are involved in cell elongation and differentiation as well as in fruit ripening (reviewed in Campbell, P. and Braam, J. (1999) Trends in Plant Sci. 4: 361-366). Expansins and endoglucanases, together with xyloglucan endotransglycosyltases, play important roles during cell wall growth (reviewed in Cosgrove, D. J. (1999) Annual Review Plant Physiol. Plant Mol. Biol. 50: 391-417 and McQueen-Mason, S. J., Rochange, F. (1999) Plant Biology 1: 19-25).

Extensin is certainly the best studied plant cell wall structural protein. It forms a multigene family, with different isoforms localized in different cell wall types and connected to different components of the cell wall. The function of extensins is not yet clear, however, some isoforms play a significant role in development, wound healing, and plant defense (reviewed in Cassab, G. I. (1998) Annu. Rev. Plant Physiol. Plant Mol. Biol. 49: 281-309).

Soluble sugars

The synthesis of soluble sugars starts with the assimilation of carbon in the reductive pentose phosphate cycle (Calvin-Benson Cycle) localized in the plastids. For reference about Calvin-Benson Cycle see e.g. Woodrow, I. E. and Berry, J. A. (1988) Ann. Rev. Plant Physiol. Plant Mol. Biol. 39: 533-594. It has to be dated that many of the sugar metabolism pathways are linked and interconnected. For review and description of such cycles suchas the tricarbonic acid cycle, glycolysis and respiration see in: Plant Physiology, Biochemistry and Molecular Biology, eds.: Dennis & Turpin; Longman Scientific & Technical, Longman House, Burnt Mill, Harlow UK, 2^{nd} edition: the whole book.

C4-plants utilize a distinctive feature to increase the CO2 concentration in the plastids: the maltate/pyruvate shuttle system (see e.g. Furbank, R. T., Taylor, W. C. (1995) Plant Cell 7: 797-807; Schnarrenberger (1997) Curr. Genet. 32: 1-18). Genetic manipulation of enzymes of the Calvin-Benson as well as of the tricarboxylic acid cycle may be used to increase productivity of the photosynthetic machinery.

The intermediate of the Calvin-Benson Cycle fructose-1,6-bisphosphate is dephosphorylated to fructose-6-phosphate by the enzyme fructose-1,6-bisphosphate phosphatase (FBPase). Antisense inhibition of FBPase activity in potato plants leads to a dramatic reduction of the photosynthetic capacity resulting in altered metabolite levels (Kossmann, J. et al. (1992) Planta 188: 7-12). Fructose-6-phosphate is then converted into glucose-6-phosphate by phosphoglucose isomerase (hexose isomerase) and finally to glucose-1-phosphate by phosphoglucomutase (see Fridlyand, L. E., Scheibe, R. (1999), Biosystems 51: 79-93). Glucose-phosphate is utilized for starch synthesis or is transported into the cytosol via glucose-phosphate translocators.

Starch degradation results in the formation of hexose phosphates and glucose. While glucose can be exported into the cytosol via a glucose translocator (Herold et al 1981, Plant Physiol., 67:85-88; Trethewey & apRees, 1994, Biochem J. 301:449-454), hexose phosphates are converted to triose phosphates and exported into the cytosol via the triose phosphate translocator. Here glucose can be metabolized to pyruvate via the glycolytic pathway or can be converted to di- and oligosaccharides, mainly sucrose. Sucrose is the major form in which carbohydrates are translocated form source tissue to sink organs (described e.g. in Heldt, H. W. (1996) Pflanzenbiochemie, Spektrum Akademischer Verlag, Heidelberg).

The first step of sucrose biosynthesis is the formation of UDP-glucose by the enzyme UDP-glucose pyrophosphorylase (also named glucose-1-phosphate uridylyltransferase) reaction. Sucrose-6-phosphate is formed in an irreversible translocation of the glucose residue to fructose-6-phospate by the sucrose-phosphate synthase (or UDP-glucose-fructosephosphate glucosyltransferase). Sucrose is formed in the irreversible sucrose phosphate phosphorylase reaction.

Fructose-1,6-bisphosphate is synthesized in the fructose-bisphosphate-aldolase reaction from triosephosphate mainly dihydroxyacetone-phosphate. Dihydroxyacetonephosphate is translocated from plastids into the cytosol via an exchange reaction of the triosephosphate-translocator, transporting inorganic phosphate into the plastids. Fructose-1,6-phosphate is dephosphorylated into fructose-6-phosphate. Fructose-6-phosphate can be converted into glucose-6-phosphate by the hexosephosphate isomerase (or phosphogluco mutase) reversible reaction or it can be utilized for sucrose synthesis as described above.

The sucrose biosynthesis pathway is highly regulated. The first committed step is the fructose-1,6-bisphosphatase reaction. This enzyme controls the flux of triosephosphate, used in the Calvin-Benson Cycle, into sucrose. An important regulator of this reaction is fructose-2,6-bisphosphate that differs from fructose-1,6-phosphate just in the position of one phosphate group. To control triosephosphate flux into sucrose synthesis, fructose-2,6-bisphosphate inhibits the synthesis of fructose-6-phosphate when the triosephosphate concentration is low (for review see: Okar D A, Lange A J. (1999) Biofactors 10: 1-14).

Another regulatory step of the sucrose synthesis is the sucrose phosphate synthase reaction. Two regulatory mechanisms are active: first the enzyme is activated by glucose-6-phosphate and inhibited by phosphate. Secondly the enzyme is phosphorylated and thereby inhibited by the sucrose-phosphate-synthase kinase and dephosphorylated by the sucrose-phosphate-synthase (further details are described by: Huber et al. (1994) International Reviews of Cytology 149: 47-98).

Sucrose is degraded in sink tissue where sucrose is utilized as an energy source or for the formation of cell walls. Cleavage of the o-glycosidic bond of sucrose is catalyzed in plants by two enzymes with entirely different properties: different isoforms of invertases and sucrose synthases. Invertases are hydrolases which cleave sucrose into fructose and glucose, whereas the sucrose synthase is a glycosyl transferase, which converts sucrose into UDP-glucose and fructose in the presence of UDP.

Another disaccharide found in plants is trehalose. Because trehalose is a stabilizing agent, it can be utilized to confer dessication and cold tolerance to plants (Hohnstöm et al. (1996) Nature 379: 683-684; Romero et al. (1997) Planta 201: 293-297). The synthesis of trehalose is very similar to that of sucrose.

Trehalose-6-phosphate is formed from UDP-glucose and glucose-6-phosphate by the enzyme trehalose-6-phosphate synthase Trehalose-phosphate phosphatase than forms trehalose (Goddijn O. J. M. and van Dun, K. (1999) Trends in Plant Science 4: 315-319). Trehalose is cleaved into two glucose molecules by the enzyme alpha,alpha-Trehalase. Beside sucrose and trehalose, raffinose, stachyose and verbascose as well as sugar-alcohol's are important transport-forms of carbohydrates (Zimmermann et al (1975) Encyclopedia of Plant Physiology, Vol I, Süringer Verlag Heidelberg: pp. 480-503). Raffinose is synthesized by the enzymes galactiol synthase and raffinose synthase. Raffinose and stachyose synthetic enzymes have been described from several plants (see e.g. Peterbauer, T. and Richter, A. (1998) Plant Physiol. 117: 165-172.

Elements and Methods of the Invention

The present invention is based, at least in part, on the discovery of novel molecules, referred to herein as CMRP nucleic acid and protein molecules, which control the construction of carbohydrates in Physcomitrella patens and Ceratodon purpureus. In one embodiment, the CMRP molecules participate in the metabolism of compounds necessary for the construction of carbohydrates in microorganisms and plants. In a preferred embodiment, the activity of the CMRP molecules of the present invention to regulate carbohydrate production has an impact on the production of a desired fine chemical by this organism. In a particularly preferred embodiment, the CMRP molecules of the invention are modulated in activity, such that the microorganisms or plants metabolic pathways which the CMRPs of the invention regulate are modulated in yield, production, and/or efficiency of production and the transport of compounds through the membranes is altered in efficiency, which either directly or indirectly modulates the yield, production, and/or efficiency of production of a desired fine chemical by microorganisms and plants.

The language, CMRP or CMRP polypeptide includes proteins which participate in the metabolism of compounds necessary for the construction of carbohydrate in microorganisms and plants. Examples of CMRPs include those encoded by the CMRP genes set forth in Table 1 and Appendix A (SEQ ID NO:1 to SEQ ID NO:177. odd integers). The terms CMRP gene or CMRP nucleic acid sequence include nucleic acid sequences encoding an CMRP, which consist of a coding region and also corresponding untranslated 5′ and 3′ sequence regions. Examples of CMRP genes include those set forth in Table 1. The terms production or productivity are art-recognized and include the concentration of the fermentation product (for example, the desired fine chemical) formed within a given time and a given fermentation volume (e.g., kg product per hour per liter). The term efficiency of production includes the time required for a particular level of production to be achieved (for example, how long it takes for the cell to attain a particular rate of output of a fine chemical). The term yield or product/carbon yield is art-recognized and includes the efficiency of the conversion of the carbon source into the product (i.e., fine chemical). This is generally written as, for example, kg product per kg carbon source. By increasing the yield or production of the compound, the quantity of recovered molecules, or of useful recovered molecules of that compound in a given amount of culture over a given amount of time is increased. The terms biosynthesis or a biosynthetic pathway are art-recognized and include the synthesis of a compound, preferably an organic compound, by a cell from intermediate compounds in what may be a multistep and highly regulated process. The terms degradation or a degradation pathway are art-recognized and include the breakdown of a compound, preferably an organic compound, by a cell to degradation products (generally speaking, smaller or less complex molecules) in what may be a multistep and highly regulated process. The language metabolism is art-recognized and includes the totality of the biochemical reactions that take place in an organism. The metabolism of a particular compound, then, (e.g., the metabolism of a carbohydrate) comprises the overall biosynthetic, modification, and degradation pathways in the cell related to this compound.

In another embodiment, the CMRP molecules of the invention are capable of modulating the production of a desired molecule, such as a fine chemical, in a microorganisms and plants. There are a number of mechanisms by which the alteration of an CMRP of the invention may directly affect the yield, production, and/or efficiency of production of a fine chemical from a microorganisms or plant strain incorporating such an altered protein. Those CMRPs involved in the transport of fine chemical molecules within or from the cell may be increased in number or activity such that greater quantities of these compounds are transported across mebranes, from which they are more readily recovered and interconverted. Similarly, those CMRPs involved in the import of nutrients necessary for the biosynthesis of one or more fine chemicals may be increased in number or activity such that these precursor, cofactor, or intermediate compounds are increased in concentration within a desired cell. Further, carbohydrates themselves are desirable fine chemicals; by optimizing the activity or increasing the number of one or more CMRPs of the invention which participate in the biosynthesis of these compounds, or by impairing the activity of one or more CMRPs which are involved in the degradation of these compounds, it may be possible to increase the yield, production, and/or efficiency of production of carbohydrates from microorganisms or plants.

The mutagenesis of one or more CMRP genes of the invention may also result in CMRPs having altered activities which indirectly impact the production of one or more desired fine chemicals from microorganisms and plants. For example, CMRPs of the invention involved in the export of waste products may be increased in number or activity such that the normal metabolic wastes of the cell (possibly increased in quantity due to the overproduction of the desired fine chemical) are efficiently exported before they are able to damage nucleotides and proteins within the cell (which would decrease the viability of the cell) or to interfere with fine chemical biosynthetic pathways (which would decrease the yield, production, or efficiency of production of the desired fine chemical). Further, the relatively large intracellular quantities of the desired fine chemical may in itself be toxic to the cell, so by increasing the activity or number of transporters able to export this compound from the cell, one may increase the viability of the cell in culture, in turn leading to a greater number of cells in the culture producing the desired fine chemical. The CMRPs of the invention may also be manipulated such that the relative amounts of different carbohydrate molecules are produced. This may have a profound effect on the sugar composition of the polysaccharides of the cell (e.g. starch and cell wall polysaccharides). Since each type of polysaccharide has different physical properties, an alteration in the sugar composition or in the chain length of a polysaccharide may significantly alter its physical properties. In the case of cell wall polysaccharides this can impact the stability and flexibility of the cell wall which in turn may result in altered growth and yield as well as in altered tolerance towards salt, drought, heat, cold, pathogens like bacteria and fungi. An altered tolerance towards drought can also be expected by altering the content of oligosaccharides like trehalose and raffinose in plants. Modulating plant carbohydrates therefore can have a profound effect on the plants fitness to survive under aforementioned stress parameters. This can happen either via the changed content, composition and structure of carbohydrates (e.g. see Arioli, T. et al. (1998) Science 279: 717-720 and Reiter, W. -D. (1998) Trends in Plants Sciene 3: 27-32) or via an altered carbohydrate partitioning (reviewed in Siedow, J. N. and Stitt, M. (1998) Current Opinion in Plant Biology 1: 197-200 and in Sturm, A. and Tang, G. -Q. (1999) Trends in Plant Science 4: 401-407).

The isolated nucleic acid sequences of the invention are contained within the genome of a Physcomitrella patens strain available through the moss collection of the University of Hamburg. The nucleotide sequence of the isolated Physcomitrella patens CMRP cDNAs and the predicted amino acid sequences of the Physcomitrella patens CMRPs are shown in Appendices A and B, respectively.

The present invention also pertains to proteins which have an amino acid sequence which is substantially homologous to an amino acid sequence of Appendix B (SEQ ID NO:2 to SEQ ID NO:178, even integers). As used herein, a protein which has an amino acid sequence which is substantially homologous to a selected amino acid sequence is least about 50% homologous to the selected amino acid sequence, e.g., the entire selected amino acid sequence. A protein which has an amino acid sequence which is substantially homologous to a selected amino acid sequence can also be at least about 50-60%, preferably at least about 60-70%, and more preferably at least about 70-80%, 80-90%, or 90-95%, and most preferably at least about 96%, 97%, 98%, 99% or more homologous to the selected amino acid sequence.

The CMRP or a biologically active portion or fragment thereof of the invention can participate in the metabolism of compounds necessary for the construction of carbohydrates in microorganisms or plants, or in the transport of sugar metabolites across these membranes, or have one or more of the activities set forth in Table 1.

Various aspects of the invention are described in further detail in the following subsections:

A. Isolated Nucleic Acid Molecules

One aspect of the invention pertains to isolated nucleic acid molecules that encode CMRP polypeptides or biologically active portions thereof, as well as nucleic acid fragments sufficient for use as hybridization probes or primers for the identification or amplification of CMRP-encoding nucleic acid (e.g., CMRP DNA). As used herein, the term “nucleic acid molecule” is intended to include DNA molecules (e.g., cDNA or genomic DNA) and RNA molecules (e.g., mRNA) and analogs of the DNA or RNA generated using nucleotide analogs. This term also encompasses untranslated sequence located at both the 3′ and 5′ ends of the coding region of the gene: at least about 100 nucleotides of sequence upstream from the 5′ end of the coding region and at least about 20 nucleotides of sequence downstream from the 3′ end of the coding region of the gene. The nucleic acid molecule can be single-stranded or double-stranded, but preferably is double-stranded DNA. An “isolated” nucleic acid molecule is one which is separated from other nucleic acid molecules which are present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated CMRP nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived (e.g., a Physcomitrella patens cell). Moreover, an “isolated” nucleic acid molecule, such as a cDNA molecule, can be substantially free of other cellular material, or culture medium when produced by recombinant techniques, or chemical precursors or other chemicals when chemically synthesized.

A nucleic acid molecule of the present invention, e.g., a nucleic acid molecule having a nucleotide sequence of Appendix A (SEQ ID NO:1 to SEQ ID NO:177, odd integers), or a portion thereof, can be isolated using standard molecular biology techniques and the sequence information provided herein. For example, a P. patens CMRP cDNA can be isolated from a P. patens library using all or portion of one of the sequences of Appendix A (SEQ ID NO:1 to SEQ ID NO:177, odd integers) as a hybridization probe and standard hybridization techniques (e.g., as described in Sambrook et al., Molecular Cloning: A Laboratory Manual. 2nd, ed., Cold Spring Harbor Laboratory, Cold Spring Harbor Laboratory Press, Cold Spring Harbor, N.Y., 1989). Moreover, a nucleic acid molecule encompassing all or a portion of one of the sequences of Appendix A (SEQ ID NO:1 to SEQ ID NO:177, odd integers) can be isolated by the polymerase chain reaction using oligonucleotide primers designed based upon this sequence (e.g., a nucleic acid molecule encompassing all or a portion of one of the sequences of Appendix A (SEQ ID NO:1 to SEQ ID NO:177, odd integers) can be isolated by the polymerase chain reaction using oligonucleotide primers designed based upon this same sequence of Appendix A (SEQ ID NO:1 to SEQ ID NO:177, odd integers)). For example, mRNA can be isolated from plant cells (e.g., by the guanidinium-thiocyanate extraction procedure of Chirgwin et al. (1979) Biochemistry 18: 5294-5299) and cDNA can be prepared using reverse transcriptase (e.g., Moloney MLV reverse transcriptase, available from Gibco/BRL, Bethesda, Md.; or AMV reverse transcriptase, available from Seikagaku America, Inc., St. Petersburg, Fla.). Synthetic oligonucleotide primers for polymerase chain reaction amplification can be designed based upon one of the nucleotide sequences shown in Appendix A (SEQ ID NO:1 to SEQ ID NO:177, odd integers). A nucleic acid of the invention can be amplified using cDNA or, alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to an CMRP nucleotide sequence can be prepared by standard synthetic techniques, e.g., using an automated DNA synthesizer.

In a preferred embodiment, an isolated nucleic acid molecule of the invention comprises one of the nucleotide sequences shown in Appendix A (SEQ ID NO:1 to SEQ ID NO:177, odd integers). The sequences of Appendix A (SEQ ID NO:1 to SEQ ID NO:177, odd integers) correspond to the Physcomitrella patens CMRP cDNAs of the invention. This cDNA comprises sequences encoding CMRPs (i.e., the “coding region”, indicated in each sequence in Appendix A (SEQ ID NO:1 to SEQ ID NO:177, odd integers)), as well as 5′ untranslated sequences and 3′ untranslated sequences. Alternatively, the nucleic acid molecule can comprise only the coding region of any of the sequences in Appendix A (SEQ ID NO:1 to SEQ ID NO:177, odd integers) or can contain whole genomic fragments isolated from genomic DNA.

For the purposes of this application, it will be understood that each of the sequences set forth in Appendix A (SEQ ID NO:1 to SEQ ID NO:177, odd integers) has an identifying entry number. Each of these sequences comprises up to three parts: a 5′ upstream region, a coding region, and a downstream region. Each of these three regions is identified by the same entry number designation to eliminate confusion. The recitation of one of the sequences in Appendix A (SEQ ID NO:1 to SEQ ID NO:177, odd integers), then, refers to any of the sequences in Appendix A (SEQ ID NO:1 to SEQ ID NO:177, odd integers), which may be distinguished by their differing entry number designations. The coding region of each of these sequences is translated into a corresponding amino acid sequence, which is set forth in Appendix B (SEQ ID NO:2 to SEQ ID NO:178, even integers). The sequences of Appendix B (SEQ ID NO:2 to SEQ ID NO:178, even integers) are identified by the same entry numbers designations as Appendix A (SEQ ID NO:1 to SEQ ID NO:177, odd integers), such that they can be readily correlated. For example, the amino acid sequence in Appendix B (SEQ ID NO:2 to SEQ ID NO:178, even integers) designated 19_ck1_d01fwd (SEQ ID NO:56) is a translation of the coding region of the nucleotide sequence of nucleic acid molecule 19_ck1_d01fwd (SEQ ID NO:55). Table 1 gives the function and utility of the respective clones as 19_ck1_d01fwd is identified as a cytosolic phosphoglucomutase.

In another preferred embodiment, an isolated nucleic acid molecule of the invention comprises a nucleic acid molecule which is a complement of one of the nucleotide sequences shown in Appendix A (SEQ I) NO:1 to SEQ ID NO:177, odd integers), or a portion thereof A nucleic acid molecule which is complementary to one of the nucleotide sequences shown in Appendix A (SEQ ID NO:1 to SEQ ID NO:177, odd integers) is one which is sufficiently complementary to one of the nucleotide sequences shown in Appendix A (SEQ ID NO:1 to SEQ ID NO:177, odd integers) such that it can hybridize to one of the nucleotide sequences shown in Appendix A (SEQ ID NO:1 to SEQ ID NO:177, odd integers), thereby forming a stable duplex.

In still another preferred embodiment, an isolated nucleic acid molecule of the invention comprises a nucleotide sequence which is at least about 50-60%, preferably at least about 60-70%, more preferably at least about 70-80%, 80-90%, or 90-95%, and even more preferably at least about 95%, 96%, 97%, 98%, 99% or more homologous to a nucleotide sequence shown in Appendix A (SEQ ID NO:1 to SEQ ID NO:177, odd integers), or a portion thereof. In an additional preferred embodiment, an isolated nucleic acid molecule of the invention comprises a nucleotide sequence which hybridizes, e.g., hybridizes under stringent conditions, to one of the nucleotide sequences shown in Appendix A (SEQ ID NO:1 to SEQ ID NO:177, odd integers), or a portion thereof

Moreover, the nucleic acid molecule of the invention can comprise only a portion of the coding region of one of the sequences in Appendix A (SEQ ID NO:1 to SEQ ID NO:177, odd integers), for example a fragment which can be used as a probe or primer or a fragment encoding a biologically active portion of an CMRP. The nucleotide sequences determined from the cloning of the CMRP genes from P. patens allows for the generation of probes and primers designed for use in identifying and/or cloning CMRP homologues in other cell types and organisms, as well as CMRP homologues from other mosses or related species. The probe/primer typically comprises substantially purified oligonucleotide. The oligonucleotide typically comprises a region of nucleotide sequence that hybridizes under stringent conditions to at least about 12, preferably about 25, more preferably about 40, 50 or 75 consecutive nucleotides of a sense strand of one of the sequences set forth in Appendix A (SEQ ID NO:1 to SEQ ID NO:177, odd integers), an anti-sense sequence of one of the sequences set forth in Appendix A (SEQ ID NO:1 to SEQ ID NO:177, odd integers), or naturally occurring mutants thereof. Primers based on a nucleotide sequence of Appendix A (SEQ ID NO:1 to SEQ ID NO:177, odd integers) can be used in PCR reactions to clone CMRP homologues. Probes based on the CMRP nucleotide sequences can be used to detect transcripts or genomic sequences encoding the same or homologous proteins. In preferred embodiments, the probe further comprises a label group attached thereto, e.g. the label group can be a radioisotope, a fluorescent compound, an enzyme, or an enzyme co-factor. Such probes can be used as a part of a genomic marker test kit for identifying cells which misexpress an CMRP, such as by measuring a level of an CMRP-encoding nucleic acid in a sample of cells, e.g., detecting CMRP mRNA levels or determining whether a genomic CMRP gene has been mutated or deleted.

In one embodiment, the nucleic acid molecule of the invention encodes a protein or portion thereof which includes an amino acid sequence which is sufficiently homologous to an amino acid sequence of Appendix B (SEQ ID NO:2 to SEQ ID NO:178, even integers) such that the protein or portion thereof maintains the ability to participate in the metabolism of compounds necessary for the construction of carbohydrates in microorganisms or plants. As used herein, the language “sufficiently homologous” refers to proteins or portions thereof which have amino acid sequences which include a minimum number of identical or equivalent (e.g., an amino acid residue which has a similar side chain as an amino acid residue in one of the sequences of Appendix B (SEQ ID NO:2 to SEQ ID NO:178, even integers)) amino acid residues to an amino acid sequence of Appendix B (SEQ ID NO:2 to SEQ ID NO:178, even integers) such that the protein or portion thereof is able to participate in the metabolism of compounds necessary for the construction of carbohydrates in microorganisms or plants, or in the transport of sugar metabolites across membranes. Protein members of such membrane component metabolic pathways or membrane transport systems, as described herein, may play a role in the production and secretion of one or more fine chemicals. Examples of such activities are also described herein. Thus, the function of an CMRP contributes either directly or indirectly to the yield, production, and/or efficiency of production of one or more fine chemicals. Examples of CMRP activities are set forth in Table 1.

In another embodiment, the protein is at least about 50-60%, preferably at least about 60-70%, and more preferably at least about 70-80%, 80-90%, 90-95%, and most preferably at least about 96%, 97%, 98%, 99% or more homologous to an entire amino acid sequence of Appendix B (SEQ ID NO:2 to SEQ ID NO:178, even integers).

Portions of proteins encoded by the CMRP nucleic acid molecules of the invention are preferably biologically active portions of one of the CMRPs. As used herein, the term “biologically active portion of an CMRP” is intended to include a portion, e.g., a domain/motif, of an CMRP that participates in the metabolism of compounds necessary for the construction of carbohydrates in microorganisms or plants, or has an activity as set forth in Table 1. To determine whether an CMRP or a biologically active portion thereof can participate in the metabolism of compounds necessary for the construction of carbohydrates in microorganisms or plants, an assay of enzymatic activity may be performed. Such assay methods are well known to those skilled in the art, as detailed in Example 8 of the Exemplification.

Additional nucleic acid fragments encoding biologically active portions of an CMRP can be prepared by isolating a portion of one of the sequences in Appendix B (SEQ ID NO:2 to SEQ ID NO:178, even integers), expressing the encoded portion of the CMRP or peptide (e.g., by recombinant expression in vitro) and assessing the activity of the encoded portion of the CMRP or peptide.

The invention further encompasses nucleic acid molecules that differ from one of the nucleotide sequences shown in Appendix A (SEQ ID NO: I to SEQ ID NO:177, odd integers) (and portions thereof) due to degeneracy of the genetic code and thus encode the same CMRP as that encoded by the nucleotide sequences shown in Appendix A (SEQ ID NO:1 to SEQ ID NO:177, odd integers). In another embodiment, an isolated nucleic acid molecule of the invention has a nucleotide sequence encoding a protein having an amino acid sequence shown in Appendix B (SEQ ID NO:2 to SEQ ID NO:178, even integers). In a still further embodiment, the nucleic acid molecule of the invention encodes a full length Physcomitrella patens protein which is substantially homologous to an amino acid sequence of Appendix B (SEQ ID NO:2 to SEQ ID NO:178, even integers) (encoded by an open reading frame shown in Appendix A (SEQ ID NO:1 to SEQ ID NO:177, odd integers)).

In addition to the Physcomitrella patens CMRP nucleotide sequences shown in Appendix A (SEQ ID NO:1 to SEQ ID NO:177, odd integers), it will be appreciated by those skilled in the art that DNA sequence polymorphisms that lead to changes in the amino acid sequences of CMRPs may exist within a population (e.g., the Physcomitrella patens population). Such genetic polymorphism in the CMRP gene may exist among individuals within a population due to natural variation. As used herein, the terms “gene” and “recombinant gene” refer to nucleic acid molecules comprising an open reading frame encoding an CMRP, preferably a Physcomitrella patens CMRP. Such natural variations can typically result in 1-5% variance in the nucleotide sequence of the CMRP gene. Any and all such nucleotide variations and resulting amino acid polymorphisms in CMRP that are the result of natural variation and that do not alter the functional activity of CMRPs are intended to be within the scope of the invention.

Nucleic acid molecules corresponding to natural variants and non-Physcomitrella patens homologues of the Physcomitrella patens CMRP cDNA of the invention can be isolated based on their homology to Physcomitrella patens CMRP nucleic acid disclosed herein using the Physcomitrella patens cDNA, or a portion thereof, as a hybridization probe according to standard hybridization techniques under stringent hybridization conditions. Accordingly, in another embodiment, an isolated nucleic acid molecule of the invention is at least 15 nucleotides in length and hybridizes under stringent conditions to the nucleic acid molecule comprising a nucleotide sequence of Appendix A (SEQ ID NO:1 to SEQ ID NO:177, odd integers). In other embodiments, the nucleic acid is at least 30, 50, 100, 250 or more nucleotides in length. As used herein, the term “hybridizes under stringent conditions” is intended to describe conditions for hybridization and washing under which nucleotide sequences at least 60% homologous to each other typically remain hybridized to each other. Preferably, the conditions are such that sequences at least about 65%, more preferably at least about 70%, and even more preferably at least about 75% or more homologous to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6×sodium chloride/sodium citrate (SSC) at about 45° C., followed by one or more washes in 0.2×SSC, 0.1% SDS at 50-65° C. Preferably, an isolated nucleic acid molecule of the invention that hybridizes under stringent conditions to a sequence of Appendix A (SEQ ID NO:1 to SEQ ID NO:177, odd integers) corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein). In one embodiment, the nucleic acid encodes a natural Physcomitrella patens CMRP.

In addition to naturally-occurring variants of the CMRP sequence that may exist in the population, the skilled artisan will further appreciate that changes can be introduced by mutation into a nucleotide sequence of Appendix A (SEQ ID NO:1 to SEQ ID NO:177, odd integers), thereby leading to changes in the amino acid sequence of the encoded CMRP, without altering the functional ability of the CMRP. For example, nucleotide substitutions leading to amino acid substitutions at “non-essential” amino acid residues can be made in a sequence of Appendix A (SEQ ID NO:1 to SEQ ID NO:177, odd integers). A “non-essential” amino acid residue is a residue that can be altered from the wild-type sequence of one of the CMRPs (Appendix B (SEQ ID NO:2 to SEQ ID NO:178, even integers)) without altering the activity of said CMRP, whereas an “essential” amino acid residue is required for CMRP activity. Other amino acid residues, however, (e.g., those that are not conserved or only semi-conserved in the domain having CMRP activity) may not be essential for activity and thus are likely to be amenable to alteration without altering CMRP activity.

Accordingly, another aspect of the invention pertains to nucleic acid molecules encoding CMRPs that contain changes in amino acid residues that are not essential for CMRP activity. Such CMRPs differ in amino acid sequence from a sequence contained in Appendix B (SEQ ID NO:2 to SEQ ID NO:178, even integers) yet retain at least one of the CMRP activities described herein. In one embodiment, the isolated nucleic acid molecule comprises a nucleotide sequence encoding a protein, wherein the protein comprises an amino acid sequence at least about 50% homologous to an amino acid sequence of Appendix B (SEQ ID NO:2 to SEQ ID NO:178, even integers) and is capable of participation in the metabolism of compounds necessary for the construction of carbohydrates in P. patens, or has one or more activities set forth in Table 1. Preferably, the protein encoded by the nucleic acid molecule is at least about 50-60% homologous to one of the sequences in Appendix B (SEQ ID NO:2 to SEQ ID NO:178, even integers), more preferably at least about 60-70% homologous to one of the sequences in Appendix B (SEQ ID NO:2 to SEQ ID NO:178, even integers), even more preferably at least about 70-80%, 80-90%, 90-95% homologous to one of the sequences in Appendix B (SEQ ID NO:2 to SEQ ID NO:178, even integers), and most preferably at least about 96%, 97%, 98%, or 99% homologous to one of the sequences in Appendix B (SEQ ID NO:2 to SEQ ID NO:178, even integers).

To determine the percent homology of two amino acid sequences (e.g., one of the sequences of Appendix B (SEQ ID NO:2 to SEQ ID NO:178, even integers) and a mutant form thereof) or of two nucleic acids, the sequences are aligned for optimal comparison purposes (e.g., gaps can be introduced in the sequence of one protein or nucleic acid for optimal alignment with the other protein or nucleic acid). The amino acid residues or nucleotides at corresponding amino acid positions or nucleotide positions are then compared. When a position in one sequence (e.g., one of the sequences of Appendix B (SEQ ID NO:2 to SEQ ID NO:178, even integers)) is occupied by the same amino acid residue or nucleotide as the corresponding position in the other sequence (e.g., a mutant form of the sequence selected from Appendix B (SEQ ID NO:2 to SEQ ID NO:178, even integers)), then the molecules are homologous at that position (i.e., as used herein amino acid or nucleic acid “homology” is equivalent to amino acid or nucleic acid “identity”). The percent homology between the two sequences is a function of the number of identical positions shared by the sequences (i.e., % homology=numbers of identical positions/total numbers of positions×100).

An isolated nucleic acid molecule encoding an CMRP homologous to a protein sequence of Appendix B (SEQ ID NO:2 to SEQ ID NO:178, even integers) can be created by introducing one or more nucleotide substitutions, additions or deletions into a nucleotide sequence of Appendix A (SEQ ID NO:1 to SEQ ID NO:177, odd integers) such that one or more amino acid substitutions, additions or deletions are introduced into the encoded protein. Mutations can be introduced into one of the sequences of Appendix A (SEQ ID NO:1 to SEQ ID NO:177, odd integers) by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in an CMRP is preferably replaced with another amino acid residue from the same side chain family. Alternatively, in another embodiment, mutations can be introduced randomly along all or part of an CMRP coding sequence, such as by saturation mutagenesis, and the resultant mutants can be screened for an CMRP activity described herein to identify mutants that retain CMRP activity. Following mutagenesis of one of the sequences of Appendix A (SEQ ID NO:1 to SEQ ID NO:177, odd integers), the encoded protein can be expressed recombinantly and the activity of the protein can be determined using, for example, assays described herein (see Example 8 of the Exemplification).

In addition to the nucleic acid molecules encoding CMRPs described above, another aspect of the invention pertains to isolated nucleic acid molecules which are antisense thereto. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire CMRP coding strand, or to only a portion thereof In one embodiment, an antisense nucleic acid molecule is antisense to a “coding region” of the coding strand of a nucleotide sequence encoding an CMRP. The term “coding region” refers to the region of the nucleotide sequence comprising codons which are translated into amino acid residues (e.g., the entire coding region of ,,,,, comprises nucleotides 1 to . . . ). In another embodiment, the antisense nucleic acid molecule is antisense to a “noncoding region” of the coding strand of a nucleotide sequence encoding CMRP. The term “noncoding region” refers to 5′ and 3′ sequences which flank the coding region that are not translated into amino acids (i.e., also referred to as 5′ and 3′ untranslated regions).

Given the coding strand sequences encoding CMRP disclosed herein (e.g., the sequences set forth in Appendix A (SEQ ID NO:1 to SEQ ID NO:177, odd integers)), antisense nucleic acids of the invention can be designed according to the rules of Watson and Crick base pairing. The antisense nucleic acid molecule can be complementary to the entire coding region of CMRP mRNA, but more preferably is an oligonucleotide which is antisense to only a portion of the coding or noncoding region of CMRP mRNA. For example, the antisense oligonucleotide can be complementary to the region surrounding the translation start site of CMRP mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a cell or generated in situ such that they hybridize with or bind to cellular mRNA and/or genomic DNA encoding an CMRP to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. The antisense molecule can be modified such that it specifically binds to a receptor or an antigen expressed on a selected cell surface, e.g., by linking the antisense nucleic acid molecule to a peptide or an antibody which binds to a cell surface receptor or antigen. The antisense nucleic acid molecule can also be delivered to cells using the vectors described herein. To achieve sufficient intracellular concentrations of the antisense molecules, vector constructs in which the antisense nucleic acid molecule is placed under the control of a strong prokaryotic, viral, or eukaryotic including plant promoters are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave CMRP mRNA transcripts to thereby inhibit translation of CMRP mRNA. A ribozyme having specificity for an CMRP-encoding nucleic acid can be designed based upon the nucleotide sequence of an CMRP cDNA disclosed herein (for example 19_ck1_d01fwd (SEQ ID NO:55) in Appendix A (SEQ ID NO:1 to SEQ ID NO:177, odd integers)) or on the basis of a heterologous sequence to be isolated according to methods taught in this invention. For example, a derivative of a Tetrahymena L-19 IVS RNA can be constructed in which the nucleotide sequence of the active site is complementary to the nucleotide sequence to be cleaved in an CMRP-encoding mRNA. See, e.g., Cech et al. U.S. Pat. No. 4,987,071 and Cech et al. U.S. Pat. No. 5,116,742. Alternatively, CMRP mRNA can be used to select a catalytic RNA having a specific ribonuclease activity from a pool of RNA molecules. See, e.g., Bartel, D. and Szostak, J. W. (1993) Science 261:1411-1418.

Alternatively, CMRP gene expression can be inhibited by targeting nucleotide sequences complementary to the regulatory region of an CMRP nucleotide sequence (e.g., an CMRP promoter and/or enhancers) to form triple helical structures that prevent transcription of an CMRP gene in target cells. See generally, Helene, C. (1991) Anticancer Drug Des. 6(6):569-84; Helene, C. et al. (1992) Ann. N. Y. Acad. Sci. 660:27-36; and Maher, L. J. (1992) Bioassays 14(12):807-15.

B. Recombinant Expression Vectors and Host Cells

Another aspect of the invention pertains to vectors, preferably expression vectors, containing a nucleic acid encoding an CMRP (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence are fused to each other so that both sequences fulfil the proposed function addicted to the sequence used. (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990) or see: Gruber and Crosby, in: Methods in Plant Molecular Biology and Biotechnolgy, CRC Press, Boca Raton, Fla., eds.:Glick and Thompson, Chapter 7, 89-108 including the references therein. Regulatory sequences include those which direct constitutive expression of a nucleotide sequence in many types of host cell and those which direct expression of the nucleotide sequence only in certain host cells or under certain conditions. It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., CMRPs, mutant forms of CMRPs, fusion proteins, etc.).

The recombinant expression vectors of the invention can be designed for expression of CMRPs in prokaryotic or eukaryotic cells. For example, CMRP genes can be expressed in bacterial cells such as C. glutamicum, insect cells (using baculovirus expression vectors), yeast and other fungal cells (see Romanos, M. A. et al. (1992) Foreign gene expression in yeast: a review, Yeast 8: 423-488; van den Hondel, C. A. M. J. J. et al. (1991) Heterologous gene expression in filamentous fungi, in: More Gene Manipulations in Fungi, J. W. Bennet & L. L. Lasure, eds., p. 396-428: Academic Press: San Diego; and van den Hondel, C. A. M. J. J. & Punt, P. J. (1991) Gene transfer systems and vector development for filamentous fungi, in: Applied Molecular Genetics of Fungi, Peberdy, J. F. et al., eds., p. 1-28, Cambridge University Press: Cambridge), algae (Falciatore et al., 1999, Marine Biotechnology.1, 3:239-251), ciliates of the types: Holotrichia, Peritrichia, Spirotrichia, Suctoria, Tetrahymena, Paramecium, Colpidium, Glaucoma, Platyophrya, Potomacus, Pseudocohnilembus, Euplotes, Engelmaniella, and Stylonychia, especially of the genus Stylonychia lemnae with vectors following a transformation method as described in WO9801572 and multicellular plant cells (see Schmidt, R. and Willlmitzer, L. (1988), High efficiency Agrobacterium tumefaciens-mediated transformation of Arabidopsis thaliana leaf and cotyledon explants, Plant Cell Rep.: 583-586); Plant Molecular Biology and Biotechnology, C Press, Boca Raton, Fla., chapter 6/7, S.71-119 (1993); F. F. White, B. Jenes et al., Techniques for Gene Transfer, in: Transgenic Plants, Vol. 1, Engineering and Utilization, eds.:Kung und R. Wu, Academic Press (1993), 128-43; Potrykus, Annu. Rev. Plant Physiol. Plant Molec. Biol. 42 (1991), 205-225 (and references cited therein) or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out with vectors containing constitutive or inducible promoters directing the expression of either fusion or non-fusion proteins. Fusion vectors add a number of amino acids to a protein encoded therein, usually to the amino terminus of the recombinant protein but also to the C-terminus or fused within suitable regions in the proteins. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase.

Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein. In one embodiment, the coding sequence of the CMRP is cloned into a pGEX expression vector to create a vector encoding a fusion protein comprising, from the N-terminus to the C-terminus, GST-thrombin cleavage site-X protein. The fusion protein can be purified by affinity chromatography using glutathione-agarose resin. Recombinant CMRP unfused to GST can be recovered by cleavage of the fusion protein with thrombin.

Examples of suitable inducible non-fusion E. coli expression vectors include pTrc (Amann et al., (1988) Gene 69:301-315) and pET lid (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMB174(DE3) from a resident λ prophage harboring a T7 gn1 gene under the transcriptional control of the lacUV 5 promoter.

One strategy to maximize recombinant protein expression is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in the bacterium chosen for expression, such as C. glutamicum (Wada et al. (1992) Nucleic Acids Res. 20:2111-2118). Such alteration of nucleic acid sequences of the invention can be carried out by standard DNA synthesis techniques.

In another embodiment, the CMRP expression vector is a yeast expression vector. Examples of vectors for expression in yeast S. cerivisae include pYepSec1 (Baldari, et al., (1987) Embo J. 6:229-234), pMFa (Kuijan and Herskowitz, (1982) Cell 30:933-943), pJRY88 (Schultz et al., (1987) Gene 54:113-123), and pYES2 (Invitrogen Corporation, San Diego, Calif.). Vectors and methods for the construction of vectors appropriate for use in other fungi, such as the filamentous fungi, include those detailed in: van den Hondel, C. A. M. J. J. & Punt, P. J. (1991) “Gene transfer systems and vector development for filamentous fungi, in: Applied Molecular Genetics of Fungi, J. F. Peberdy, et al., eds., p. 1-28, Cambridge University Press: Cambridge.

Alternatively, the CMRPs of the invention can be expressed in insect cells using baculovirus expression vectors. Baculovirus vectors available for expression of proteins in cultured insect cells (e.g., Sf9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simnian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) PNAS 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the fetoprotein promoter (Campes and Tilghman (1989) Genes Dev. 3:537-546).

In another embodiment, the CMRPs of the invention may be expressed in unicellular plant cells (such as algae) see Falciatore et al., 1999, Marine Biotechnology. 1 (3):239-251 and references therein and plant cells from higher plants (e.g., the spermatophytes, such as crop plants). Examples of plant expression vectors include those detailed in: Becker, D., Kemper, E., Schell, J. and Masterson, R. (1992) “New plant binary vectors with selectable markers located proximal to the left border”, Plant Mol. Biol. 20: 1195-1197; and Bevan, M. W. (1984) “Binary Agrobacterium vectors for plant transformation, Nucl. Acid Res. 12: 8711-8721; Vectors for Gene Transfer in Higher Plants; in: Transgenic Plants, Vol. 1, Engineering and Utilization, eds.: Kung und R. Wu, Academic Press, 1993, S. 15-38.

A plant expression cassette preferably contains regulatory sequences capable to drive gene expression in plants cells and which are operably linked so that each sequence can fulfil its function such as termination of transcription such as polyadenylation signals. Preferred polyadenylation signals are those originating from Agrobacterium tumefaciens t-DNA such as the gene 3 known as octopine synthase of the Ti-plasmid pTiACH5 (Gielen et al., EMBO J. 3 (1984), 835 ff) or functional equivalents therof but also all other terminators functionally active in plants are suitable.

As plant gene expression is very often not limited on transcriptional levels a plant expression cassette preferably contains other operably linked sequences like translational enhancers such as the overdrive-sequence containing the 5′-untranlated leader sequence from tobacco mosaic virus enhancing the protein per RNA ratio (Gallie et al 1987, Nuel. Acids Research 15:8693-8711).

Plant gene expression has to be operably linked to an appropriate promoter conferring gene expression in a timely , cell or tissue specific manner. Preferrred are promoters driving constitutitive expression (Benfey et al., EMBO J. 8 (1989) 2195-2202) like those derived from plant viruses like the 35S CAMV (Franck et al., Cell 21(1980) 285-294), the 19S CaMV (see also U.S. Pat. No. 5,352,605 and WO8402913) or plant promoters like those from Rubisco small subunit described in U.S. Pat. No. 4,962,028.

Other preferred sequences for use operable linkage in plant gene expression cassettes are targeting-sequences necessary to direct the gene-product in its appropriate cell compartment (for review see Kermode, Crit. Rev. Plant Sci. 15, 4 (1996), 285-423 and references cited therin) such as the vacuole, the nucleus, all types of plastids like amyloplasts, chloroplasts, chromoplasts, the extracellular space, mitochondria, the endoplasmic reticulum, oil bodies, peroxisomes and other compartments of plant cells.

Plant gene expression can also be facilitated via a chemically inducible promoter (for rewiew see Gatz 1997, Annu. Rev. Plant Physiol. Plant Mol. Biol., 48:89-108). Chemically inducible promoters are especially suitable if gene expression is wanted to occur in a time specific manner. Examples for such promoters are a salicylic acid inducible promoter (WO 95/19443), a tetracycline inducible promoter (Gatz et al., (1992) Plant J. 2, 397-404) and an ethanol inducible promoter (WO 93/21334).

Also promoters responding to biotic or abiotic stress conditions are suitable promoters such as the pathogen inducible PRP 1-gene promoter (Ward et al., Plant. Mol. Biol. 22 (1993), 361-366), the heat inducible hsp8o-promoter from tomato (U.S. Pat. No. 5,187,267), cold inducible alpha-amylase promoter from potato (WO9612814) or the wound-inducible pinII-promoter (EP375091).

Especially those promoters are preferred which confer gene expression in tissues and organs where lipid and oil biosynthesis occurs in seed cells such as cells of the endosperm and the developing embryo. Suitable promoters are the napin-gene promoter from rapeseed (U.S. Pat. No. 5,608,152), the USP-promoter from Vicia faba (Baeumlein et al., Mol Gen Genet, 1991, 225 (3):459-67), the oleosin-promoter from Arabidopsis (WO9845461), the phaseolin-promoter from Phaseolus vulgaris (U.S. Pat. No. 5,504,200), the Bce4-promoter from Brassica (WO9113980) or the legumin B4 promoter (LeB4; Baeumlein et al., 1992, Plant Journal, 2 (2):233-9) as well as promoters conferring seed specific expression in monocot plants like maize, barley, wheat, rye, rice etc. Suitable promoters to note are the 1pt2 or 1pt1-gene promoter from barley (WO9515389 and WO9523230) or those desribed in WO9916890 (promoters from the barley hordein-gene, the rice glutelin gene, the rice oryzin gene, the rice prolamin gene, the wheat gliadin gene, wheat glutelin gene, the maize zein gene, the oat glutelin gene, the Sorghum kasirin-gene, the rye secalin gene).

Also especially suited are promoters that confer plastid-specific gene expression as plastids are the compartment where precursors and some end products of lipid biosynthesis are synthesized. Suitable promoters such as the viral RNA-polymerase promoter are described in WO9516783 and WO9706250 and the clpP-promoter from Arabidopsis described in WO9946394.

The invention farther provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to CMRP mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986 and Mol et al., 1990, FEBS Letters 268:427-430.

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, an CMRP can be expressed in bacterial cells such as C. glutamicum, insect cells, fungal cells or mammalian cells (such as Chinese hamster ovary cells (CHO) or COS cells), algae, ciliates, plant cells, fungi or other microorganims like C. glutamicum. Other suitable host cells are known to those skilled in the art.

Vector DNA can be introduced into prokaryotic or eukaryotic cells via conventional transformation or transfection techniques. As used herein, the terms “transformation” and “transfection”, conjugation and transduction are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, natural competence, chemical-mediated transfer, or electroporation. Suitable methods for transforming or transfecting host cells including plant cells can be found in Sambrook, et al. (Molecular Cloning: A Laboratory Manual. 2nd, ed, Cold Spring Harbor Laboratory, Cold Spring Harbor Laboratory Press, Cold Spring Harbor, N.Y., 1989) and other laboratory manuals such as Methods in Molecular Biology, 1995, Vol. 44, Agrobacterium protocols, ed: Gartland and Davey, Humana Press, Totowa, N.J.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate or in plants that confer resistance towards a herbicide such as glyphosate or glufosinate. Nucleic acid encoding a selectable marker can be introduced into a host cell on the same vector as that encoding an CMRP or can be introduced on a separate vector. Cells stably transfected with the introduced nucleic acid can be identified by, for example, drug selection (e.g., cells that have incorporated the selectable marker gene will survive, while the other cells die).

To create a homologous recombinant microorganism, a vector is prepared which contains at least a portion of an CMRP gene into which a deletion, addition or substitution has been introduced to thereby alter, e.g., functionally disrupt, the CMRP gene. Preferably, this CMRP gene is a Physcomitrella patens CMRP gene, but it can be a homologue from a related plant or even from a mammalian, yeast, or insect source. In a preferred embodiment, the vector is designed such that, upon homologous recombination, the endogenous CMRP gene is functionally disrupted (i.e., no longer encodes a functional protein; also referred to as a knock-out vector). Alternatively, the vector can be designed such that, upon homologous recombination, the endogenous CMRP gene is mutated or otherwise altered but still encodes functional protein (e.g., the upstream regulatory region can be altered to thereby alter the expression of the endogenous CMRP). To create a point mutation via homologous recombination also DNA-RNA hybrids can be used known as chimeraplasty known from Cole-Strauss et al. 1999, Nucleic Acids Research 27(5):1323-1330 and Kmiec Gene therapy. 19999, American Scientist. 87(3):240-247.

Whereas in the homologous recombination vector, the altered portion of the CMRP gene is flanked at its 5′ and 3′ ends by additional nucleic acid of the CMRP gene to allow for homologous recombination to occur between the exogenous CMRP gene carried by the vector and an endogenous CMRP gene in a microorganism or plant. The additional flanking CMRP nucleic acid is of sufficient length for successful homologous recombination with the endogenous gene. Typically, several hundreds of basepairs up to kilobases of flanking DNA (both at the 5′ and 3′ ends) are included in the vector (see e.g., Thomas, K. R., and Capecchi, M. R. (1987) Cell 51: 503 for a description of homologous recombination vectors or Strepp et al., 1998, PNAS, 95 (8):4368-4373 for cDNA based recombination in Physcomitrella patens). The vector is introduced into a microorganism or plant cell (e.g., via polyethyleneglycol mediated DNA) and cells in which the introduced CMRP gene has homologously recombined with the endogenous CMRP gene are selected, using art-known techniques.

In another embodiment, recombinant microorganisms can be produced which contain selected systems which allow for regulated expression of the introduced gene. For example, inclusion of an CMRP gene on a vector placing it under control of the lac operon permits expression of the CMRP gene only in the presence of IPTG. Such regulatory systems are well known in the art.

A host cell of the invention, such as a prokaryotic or eukaryotic host cell in culture, can be used to produce (i.e., express) an CMRP. An alternate method can be applied in addition in plants by the direct transfer of DNA into developing flowers via electroporation or Agrobacterium medium gene transfer. Accordingly, the invention further provides methods for producing CMRPs using the host cells of the invention. In one embodiment, the method comprises culturing the host cell of invention (into which a recombinant expression vector encoding an CMRP has been introduced, or into which genome has been introduced a gene encoding a wild-type or altered CMRP) in a suitable medium until CMRP is produced. In another embodiment, the method further comprises isolating CMRPs from the medium or the host cell.

C. Isolated CMRPs

Another aspect of the invention pertains to isolated CMRPs, and biologically active portions thereof. An “isolated” or “purified” protein or biologically active portion thereof is substantially free of cellular material when produced by recombinant DNA techniques, or chemical precursors or other chemicals when chemically synthesized. The language “substantially free of cellular material” includes preparations of CMRP in which the protein is separated from cellular components of the cells in which it is naturally or recombinantly produced. In one embodiment, the language “substantially free of cellular material” includes preparations of CMRP having less than about 30% (by dry weight) of non-CMRP (also referred to herein as a “contaminating protein”), more preferably less than about 20% of non-CMRP, still more preferably less than about 10% of non-CMRP, and most preferably less than about 5% non-CMRP. When the CMRP or biologically active portion thereof is recombinantly produced, it is also preferably substantially free of culture medium, i.e., culture medium represents less than about 20%, more preferably less than about 10%, and most preferably less than about 5% of the volume of the protein preparation. The language “substantially free of chemical precursors or other chemicals” includes preparations of CMRP in which the protein is separated from chemical precursors or other chemicals which are involved in the synthesis of the protein. In one embodiment, the language “substantially free of chemical precursors or other chemicals” includes preparations of CMRP having less than about 30% (by dry weight) of chemical precursors or non-CMRP chemicals, more preferably less than about 20% chemical precursors or non-CMRP chemicals, still more preferably less than about 10% chemical precursors or non-CMRP chemicals, and most preferably less than about 5% chemical precursors or non-CMRP chemicals. In preferred embodiments, isolated proteins or biologically active portions thereof lack contaminating proteins from the same organism from which the CMRP is derived. Typically, such proteins are produced by recombinant expression of, for example, a Physcomitrella patens CMRP in other plants than Physcomitrella patens or microorganisms such as C. glutamicum or ciliates, algae or fungi.

An isolated CMRP or a portion thereof of the invention can participate in the metabolism of compounds necessary for the construction of carbohydrates in Physcomitrella patens, has one or more of the activities set forth in Table 1. In preferred embodiments, the protein or portion thereof comprises an amino acid sequence which is sufficiently homologous to an amino acid sequence of Appendix B (SEQ ID NO:2 to SEQ ID NO:178, even integers) such that the protein or portion thereof maintains the ability participate in the metabolism of compounds necessary for the construction of carbohydrates in Physcomitrella patens. The portion of the protein is preferably a biologically active portion as described herein. In another preferred embodiment, an CMRP of the invention has an amino acid sequence shown in Appendix B (SEQ ID NO:2 to SEQ ID NO:178, even integers). In yet another preferred embodiment, the CMRP has an amino acid sequence which is encoded by a nucleotide sequence which hybridizes, e.g., hybridizes under stringent conditions, to a nucleotide sequence of Appendix A (SEQ ID NO:1 to SEQ ID NO:177, odd integers). In still another preferred embodiment, the CMRP has an amino acid sequence which is encoded by a nucleotide sequence that is at least about 50-60%, preferably at least about 60-70%, more preferably at least about 70-80%, 80-90%, 90-95%, and even more preferably at least about 96%, 97%, 98%, 99% or more homologous to one of the amino acid sequences of Appendix B (SEQ ID NO:2 to SEQ ID NO:178, even integers). The preferred CMRPs of the present invention also preferably possess at least one of the CMRP activities described herein. For example, a preferred CMRP of the present invention includes an amino acid sequence encoded by a nucleotide sequence which hybridizes, e.g., hybridizes under stringent conditions, to a nucleotide sequence of Appendix A (SEQ ID NO:1 to SEQ ID NO:177, odd integers), and which can participate in the metabolism of compounds necessary for the construction of carbohydrates in Physcomitrella patens, or which has one or more of the activities set forth in Table 1.

In other embodiments, the CMRP is substantially homologous to an amino acid sequence of Appendix B (SEQ ID NO:2 to SEQ ID NO:178, even integers) and retains the functional activity of the protein of one of the sequences of Appendix B (SEQ ID NO:2 to SEQ ID NO:178, even integers) yet differs in amino acid sequence due to natural variation or mutagenesis, as described in detail in subsection I above. Accordingly, in another embodiment, the CMRP is a protein which comprises an amino acid sequence which is at least about 50-60%, preferably at least about 60-70%, and more preferably at least about 70-80, 80-90, 90-95%, and most preferably at least about 96%, 97%, 98%, 99% or more homologous to an entire amino acid sequence of Appendix B (SEQ ID NO:2 to SEQ ID NO:178, even integers) and which has at least one of the CMRP activities described herein. In another embodiment, the invention pertains to a full Physcomitrella patens protein which is substantially homologous to an entire amino acid sequence of Appendix B (SEQ ID NO:2 to SEQ ID NO:178, even integers).

Biologically active portions of an CMRP include peptides comprising amino acid sequences derived from the amino acid sequence of an CMRP, e.g., the an amino acid sequence shown in Appendix B (SEQ ID NO:2 to SEQ ID NO:178, even integers) or the amino acid sequence of a protein homologous to an CMRP, which include fewer amino acids than a full length CMRP or the full length protein which is homologous to an CMRP, and exhibit at least one activity of an CMRP. Typically, biologically active portions (peptides, e.g., peptides which are, for example, 5, 10, 15, 20, 30, 35, 36, 37, 38, 39, 40, 50, 100 or more amino acids in length) comprise a domain or motif with at least one activity of an CMRP. Moreover, other biologically active portions, in which other regions of the protein are deleted, can be prepared by recombinant techniques and evaluated for one or more of the activities described herein. Preferably, the biologically active portions of an CMRP include one or more selected domains/motifs or portions thereof having biological activity.

CMRPs are preferably produced by recombinant DNA techniques. For example, a nucleic acid molecule encoding the protein is cloned into an expression vector (as described above), the expression vector is introduced into a host cell (as described above) and the CMRP is expressed in the host cell. The CMRP can then be isolated from the cells by an appropriate purification scheme using standard protein purification techniques. Alternative to recombinant expression, an CMRP, polypeptide, or peptide can be synthesized chemically using standard peptide synthesis techniques. Moreover, native CMRP can be isolated from cells (e.g., endothelial cells), for example using an anti-CMRP antibody, which can be produced by standard techniques utilizing an CMRP or fragment thereof of this invention.

The invention also provides CMRP chimeric or fusion proteins. As used herein, an CMRP “chimeric protein” or “fusion protein” comprises an CMRP polypeptide operatively linked to a non-CMRP polypeptide. An “CMRP polypeptide” refers to a polypeptide having an amino acid sequence corresponding to an CMRP, whereas a “non-CMRP polypeptide” refers to a polypeptide having an amino acid sequence corresponding to a protein which is not substantially homologous to the CMRP, e.g., a protein which is different from the CMRP and which is derived from the same or a different organism. Within the fusion protein, the term “operatively linked” is intended to indicate that the CMRP polypeptide and the non-CMRP polypeptide are fused to each other so that both sequences fulfil the proposed function addicted to the sequence used. The non-CMRP polypeptide can be fused to the N-terminus or C-terminus of the CMRP polypeptide. For example, in one embodiment the fusion protein is a GST-CMRP fusion protein in which the CMRP sequences are fused to the C-terminus of the GST sequences. Such fusion proteins can facilitate the purification of recombinant CMRPs. In another embodiment, the fusion protein is an CMRP containing a heterologous signal sequence at its N-terminus. In certain host cells (e.g., mammalian host cells), expression and/or secretion of an CMRP can be increased through use of a heterologous signal sequence.

Preferably, an CMRP chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filing-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). An CMRP-encoding nucleic acid can be cloned into such an expression vector such that the fusion moiety is linked in-frame to the CMRP.

Homologues of the CMRP can be generated by mutagenesis, e.g., discrete point mutation or truncation of the CMRP. As used herein, the term “homologue” refers to a variant form of the CMRP which acts as an agonist or antagonist of the activity of the CMRP. An agonist of the CMRP can retain substantially the same, or a subset, of the biological activities of the CMRP. An antagonist of the CMRP can inhibit one or more of the activities of the naturally occurring form of the CMRP, by, for example, competitively binding to a downstream or upstream member of the cell membrane component metabolic cascade which includes the CMRP, or by binding to an CMRP which mediates transport of compounds across such membranes, thereby preventing translocation from taking place.

In an alternative embodiment, homologues of the CMRP can be identified by screening combinatorial libraries of mutants, e.g., truncation mutants, of the CMRP for CMRP agonist or antagonist activity. In one embodiment, a variegated library of CMRP variants is generated by combinatorial mutagenesis at the nucleic acid level and is encoded by a variegated gene library. A variegated library of CMRP variants can be produced by, for example, enzymatically ligating a mixture of synthetic oligonucleotides into gene sequences such that a degenerate set of potential CMRP sequences is expressible as individual polypeptides, or alternatively, as a set of larger fusion proteins (e.g., for phage display) containing the set of CMRP sequences therein. There are a variety of methods which can be used to produce libraries of potential CMRP homologues from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential CMRP sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of the CMRP coding can be used to generate a variegated population of CMRP fragments for screening and subsequent selection of homologues of an CMRP. In one embodiment, a library of coding sequence fragments can be generated by treating a double stranded PCR fragment of an CMRP coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the CMRP.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of CMRP homologues. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify CMRP homologues (Arkin and Yourvan (1992) PNAS 89:7811-7815; Delgrave et al. (1993) Protein Engineering 6(3):327-331).

In another embodiment, cell based assays can be exploited to analyze a variegated CMRP library, using methods well known in the art.

D. Uses and Methods of the Invention

The nucleic acid molecules, proteins, protein homologues, fusion proteins, primers, vectors, and host cells described herein can be used in one or more of the following methods: identification of Physcomitrella patens and related organisms; mapping of genomes of organisms related to Physcomitrella patens; identification and localization of Physcomitrella patens sequences of interest; evolutionary studies; determination of CMRP regions required for function; modulation of an CMRP activity; modulation of the metabolism of one or more carbohydrate components; modulation of the transmembrane transport of one or more compounds; and modulation of cellular production of a desired compound, such as a fine chemical.

The CMRP nucleic acid molecules of the invention have a variety of uses. First, they may be used to identify an organism as being Physcomitrella patens or a close relative thereof. Also, they may be used to identify the presence of Physcomitrella patens or a relative thereof in a mixed population of microorganisms. The invention provides the nucleic acid sequences of a number of Physcomitrella patens genes; by probing the extracted genomic DNA of a culture of a unique or mixed population of microorganisms under stringent conditions with a probe spanning a region of a Physcomitrella patens gene which is unique to this organism, one can ascertain whether this organism is present. Although Physcomitrella patens itself is not used for the commercial construction of carbohydrates, mosses are capable of synthesizing carbohydrates like monosaccharides, sucrose, trehalose, raffinose, starch, cellulose, hemicelluloses and pectins. Therefore DNA sequences related to CMRPs are especially suited to be used for carbohydrate production and modification in other organisms.

Further, the nucleic acid and protein molecules of the invention may serve as markers for specific regions of the genome. This has utility not only in the mapping of the genome, but also for functional studies of Physcomitrella patens proteins. For example, to identify the region of the genome to which a particular Physcomitrella patens DNA-binding protein binds, the Physcomitrella patens genome could be digested, and the fragments incubated with the DNA-binding protein. Those which bind the protein may be additionally probed with the nucleic acid molecules of the invention, preferably with readily detectable labels; binding of such a nucleic acid molecule to the genome fragment enables the localization of the fragment to the genome map of Physcomitrella patens, and, when performed multiple times with different enzymes, facilitates a rapid determination of the nucleic acid sequence to which the protein binds. Further, the nucleic acid molecules of the invention may be sufficiently homologous to the sequences of related species such that these nucleic acid molecules may serve as markers for the construction of a genomic map in related mosses, such as Physcomitrium piriforme or Ceratodon purpureus.

The CMRP nucleic acid molecules of the invention are also useful for evolutionary and protein structural studies. The metabolic and transport processes in which the molecules of the invention participate are utilized by a wide variety of prokaryotic and eukaryotic cells; by comparing the sequences of the nucleic acid molecules of the present invention to those encoding similar enzymes from other organisms, the evolutionary relatedness of the organisms can be assessed. Similarly, such a comparison permits an assessment of which regions of the sequence are conserved and which are not, which may aid in determining those regions of the protein which are essential for the functioning of the enzyme. This type of determination is of value for protein engineering studies and may give an indication of what the protein can tolerate in terms of mutagenesis without losing function.

Manipulation of the CMRP nucleic acid molecules of the invention may result in the production of CMRPs having functional differences from the wild-type CMRPs. These proteins may be improved in efficiency or activity, may be present in greater numbers in the cell than is usual, or may be decreased in efficiency or activity.

There are a number of mechanisms by which the alteration of an CMRP of the invention may directly affect the yield, production, and/or efficiency of production of a fine chemical incorporating such an altered protein. Recovery of fine chemical compounds from large-scale cultures of C. glutamicum, algae or fungi is significantly improved if the cell secrets the desired compounds, since such compounds may be readily purified from the culture medium (as opposed to extracted from the mass of cultured cells). In the case of plants expressing CMRPs increased transport can lead to improved partitioning within the plant tissue and organs. By either increasing the number or the activity of transporter molecules which export fine chemicals from the cell, it may be possible to increase the amount of the produced fine chemical which is present in the extracellular medium, thus permitting greater ease of harvesting and purification or in case of plants mor efficient partitioning. Conversely, in order to efficiently overproduce one or more fine chemicals, increased amounts of the cofactors, precursor molecules, and intermediate compounds for the appropriate biosynthetic pathways are required. Thereforee, by increasing the number and/or activity of transporter proteins involved in the import of nutrients, such as carbon sources (i.e., sugars), nitrogen sources (i.e., amino acids, ammonium salts), phosphate, and sulfur, it may be possible to improve the production of a fine chemical, due to the removal of any nutrient supply limitations on the biosynthetic process. Further, carbohydrates are themselves desirable fine chemicals, so by optimizing the activity or increasing the number of one or more CMRPs of the invention which participate in the biosynthesis of these compounds, or by impairing the activity of one or more CMRPs which are involved in the degradation of these compounds, it may be possible to increase the yield, production, and/or efficiency of production of carbohydrates in algae, plants, fungi or other microorganims like C. glutamicum.

The engineering of one or more CMRP genes of the invention may also result in CMRPs having altered activities which indirectly impact the production of one or more desired fine chemicals from algae, plants or fungi or other microorganims like C. glutamicum. For example, the normal biochemical processes of metabolism result in the production of a variety of waste products (e.g., hydrogen peroxide and other reactive oxygen species) which may actively interfere with these same metabolic processes (for example, peroxynitrite is known to nitrate tyrosine side chains, thereby inactivating some enzymes having tyrosine in the active site (Groves, J. T. (1999) Curr. Opin. Chem. Biol. 3(2): 226-235). While these waste products are typically excreted, cells utilized for large-scale fermentative production are optimized for the overproduction of one or more fine chemicals, and thus may produce more waste products than is typical for a wild-type cell. By optimizing the activity of one or more CMRPs of the invention which are involved in the export of waste molecules, it may be possible to improve the viability of the cell and to maintain efficient metabolic activity. Also, the presence of high intracellular levels of the desired fine chemical may actually be toxic to the cell, so by increasing the ability of the cell to secrete these compounds, one may improve the viability of the cell.

Further, the CMRPs of the invention may be manipulated such that the relative amounts of various carbohydrate molecules produced are altered. Especially in the case of polysaccharides this may have a profound effect on the stability and flexibility of the cell. Since each type of polysaccharide has different physical properties, and some polysaccharides are connected with each another, an alteration in the composition and of the chain length may significantly alter cell stability. By manipulating CMRPs involved in the production of carbohydrates such that the resulting carbohydrates has a sugar composition and physical property more amenable to the environmental conditions, a greater proportion of the cells should survive and multiply. Greater numbers of producing cells should translate into greater yields, production, or efficiency of production of the fine chemical from the culture.

The aforementioned mutagenesis strategies for CMRPs to result in increased yields of a fine chemical are not meant to be limiting; variations on these strategies will be readily apparent to one skilled in the art. Using such strategies, and incorporating the mechanisms disclosed herein, the nucleic acid and protein molecules of the invention may be utilized to generate algae, plants, fungi or other microorganims like C. glutamicum expressing mutated CMRP nucleic acid and protein molecules such that the yield, production, and/or efficiency of production of a desired compound is improved. This desired compound may be any natural product of algae, plants, fungi or C. glutamicum, which includes the final products of biosynthesis pathways and intermediates of naturally-occurring metabolic pathways, as well as molecules which do not naturally occur in the metabolism of said cells, but which are produced by a said cells of the invention.

This invention is further illustrated by the following examples which should not be construed as limiting. The contents of all references, patent applications, patents, and published patent applications cited throughout this application are hereby incorporated by reference.

### EXAMPLIFICATION

### Example 1

General processes

a) General cloning processes:

Cloning processes such as, for example, restriction cleavages, agarose gel electrophoresis, purification of DNA fragments, transfer of nucleic acids to nitrocellulose and nylon membranes, linkage of DNA fragments, transformation of Escherichia coli and yeast cells, growth of bacteria and sequence analysis of recombinant DNA were carried out as described in Sambrook et al. (1989) (Cold Spring Harbor Laboratory Press: ISBN 0-87969-309-6) or Kaiser, Michaelis and Mitchell (1994) ,Methods in Yeast Genetics” (Cold Spring Harbor Laboratory Press: ISBN 0-87969-451-3). Transformation and cultivation of bacteria such as Acetobacter xylimum and algae such as Chlorella are performed as described by Hall et al., Plasmid 28: 194-200 (1992) and El-Sheekh (1999) Biologia Plantarum 42: 209-216, respectively.

b)Chemicals:

The chemicals used were obtained, if not mentioned otherwise in the text, in p.a. quality from the companies Fluka (Neu-Ulm), Merck (Darmstadt), Roth (Karlsruhe), Serva (Heidelberg) and Sigma (Deisenhofen). Solutions were prepared using purified, pyrogen-free water, designated as H₂O in the following text, from a Milli-Q water system water purification plant (Millipore, Eschborn). Restriction endonucleases, DNA-modifying enzymes and molecular biology kits were obtained from the companies AGS (Heidelberg), Amersham (Braunschweig), Biometra (Gottingen), Boehringer Iannheim), Genomed (Bad Oeynnhausen), New England Biolabs (Schwalbach/Taunus), Novagen (Madison, Wis., USA), Perkin-Elmer (Weiterstadt), Pharmacia (Freiburg), Qiagen (Rilden) and Stratagene (Amsterdam, Netherlands). They were used, if not mentioned otherwise, according to the manufacturer's instructions.

c)Plant material

For this study, plants of the species Physcomitrella patens (Hedw.) B. S. G. from the collection of the genetic studies section of the University of Hamburg were used. They originate from the strain 16/14 collected by H. L. K. Whitehouse in Gransden Wood, Huntingdonshire (England), which was subcultured from a spore by Engel (1968, Am J Bot 55, 438-446). Proliferation of the plants was carried out by means of spores and by means of regeneration of the gametophytes. The protonema developed from the haploid spore as a chloroplast-rich chloronema and chloroplast-low caulonema, on which buds formed after approximately 12 days. These grew to give gametophores bearing antheridia and archegonia. After fertilization, the diploid sporophyte with a short seta and the spore capsule resulted, in which the meiospores mature.

d) Plant growth

Culturing was carried out in a climatic chamber at an air temperature of 25° C. and light intensity of 55 micromol s⁻¹ m⁻² (white light; Philips TL 65W/25 fluorescent tube) and a light/dark change of 16/8 hours. The moss was either modified in liquid culture using Knop medium according to Reski and Abel (1985, Planta 165, 354-358) or cultured on Knop solid medium using 1% oxoid agar (Unipath, Basingstoke, England).

The protonemas used for RNA and DNA isolation were cultured in aerated liquid cultures. The protonemas were comminuted every 9 days and transferred to fresh culture medium.

### Example 2

Total DNA isolation from plants

The details for the isolation of total DNA relate to the working up of one grain fresh weight of plant material.

CTAB buffer: 2% (w/v) N-cethyl-N,N,N-trimethylammonium bromide
(CTAB); 100 mM Tris HCl pH 8.0; 1.4 M NaCl; 20 mM EDTA.

N-Laurylsarcosine buffer: 10% (w/v) N-laurylsarcosine; 100 MM Tris HCl pH 8.0; 20 mM EDTA.

The plant material was triturated under liquid nitrogen in a mortar to give a fine powder and transferred to 2 ml Eppendorf vessels. The frozen plant material was then covered with a layer of 1 ml of decomposition buffer (1 ml CTAB buffer, 100 ml of N-laurylsarcosine buffer, 20 ml of b-mercaptoethanol and 10 ml of proteinase K solution, 10 mg/ml) and incubated at 60° C. for one hour with continuous shaking. The homogenate obtained was distributed into two Eppendorf vessels (2 ml) and extracted twice by shaking with the same volume of chloroform/isoamyl alcohol (24:1). For phase separation, centrifugation was carried out at 8000×g and RT for 15 min in each case.

The DNA was then precipitated at −70° C. for 30 min using ice-cold isopropanol. The precipitated DNA was sedimented at 4° C. and 10,000 g for 30 min and resuspended in 180 ml of TE buffer (Sambrook et al., 1989, Cold Spring Harbor Laboratory Press: ISBN 0-87969-309-6). For further purification, the DNA was treated with NaCl (1.2 M final concentration) and precipitated again at −70° C. for 30 min using twice the volume of absolute ethanol. After a washing step with 70% ethanol, the DNA was dried and subsequently taken up in 50 ml of H₂O+RNAse A (50 mg/ml final concentration). The DNA was dissolved overnight at 4° C. and the RNAse digestion was subsequently carried out at 37° C. for 1 h. Storage of the DNA took place at 4° C.

### Example 3

Isolation of total RNA and poly-(A)⁺ RNA from plants

For the investigation of transcripts, both total RNA and poly-(A)⁺ RNA were isolated. The total RNA was obtained from wild-type 9d old protonemata following the GTC-method (Reski et al. 1994, Mol. Gen. Genet., 244:352-359).

Isolation of poly(A)+RNA was isolated using Dyna Beads^{R} (Dynal, Oslo). Following the instructions of the manufacturers protocol.

After determination of the concentration of the RNA or of the poly(A)+RNA, the RNA was precipitated by addition of 1/10 volumes of 3 M sodium acetate pH 4.6 and 2 volumes of ehanol and stored at −70° C.

### Example 4

cDNA library construction

For cDNA library construction first strand synthesis was achieved using Murine Leukemia Virus reverse transcriptase (Roche, Mannheim, Germany) and olido-d(T)-primers, second strand synthesis by incubation with DNA polymerase I, Klenow enzyme and RNAseH digestion at 12° C. (2 h), 16° C. (1 h)) and 22° C. (1 h). The reaction was stopped by incubation at 65° C. (10 min) and subsequently transferred to ice. Double stranded DNA molecules were blunted by T4-DNA-polymerase (Roche, Mannheim) at 37° C. (30 min). Nucleotides were removed by phenol/chloroform extraction and Sephadex G50 spin columns. EcoRI adapters (Pharmacia, Freiburg, Germany) were ligated to the cDNA ends by T4-DNA-ligase (Roche, 12° C., overnight) and phosphorylated by incubation with polynucleotide kinase (Roche, 37° C., 30 min). This mixture was subjected to separation on a low melting agarose gel. DNA molecules larger than 300 basepairs were eluted from the gel, phenol extracted, concentrated on Elutip-D-columns (Schleicher and Schuell, Dassel, Germany) and were ligated to vector arms and packed into lambda ZAPII phages or lambda ZAP-Express phages using the Gigapack Gold Kit (Stratagene, Amsterdam, Netherlands) using material and following the instructions of the manufacturer.

### Example 5

Identification of genes of interest

Gene sequences can be used to identify homologous or heterologous genes from cDNA or genomic libraries.

Homologous genes (e. g. full length cDNA clones) can be isolated via nucleic acid hybridization using for example cDNA libraries: Depended on the abundance of the gene of interest 100,000 up to 1,000,000 recombinant bacteriophages are plated and transferred to a nylon membrane. After denaturation with alkali, DNA is immobilized on the membrane by e. g. UV cross linking. Hybridization is carried out at high stringency conditions. In aqueous solution hybridization and washing is performed at an ionic strength of 1 M NaCl and a temperature of 68° C. Hybridization probes are generated by e. g. radioactive (³²P) nick transcription labeling (Amersham Ready Prime). Signals are detected by exposure to x-ray films.

Partially homologous or heterologous genes that are related but not identical can be identified analog to the above described procedure using low stringency hybridization and washing conditions. For aqueous hybridization the ionic strength is normally kept at 1 M NaCl while the temperature is progressively lowered from 68 to 42° C.

Isolation of gene sequences with homologies only in a distinct domain of (for example 20 aminoacids) can be carried out by using synthetic radioactively labeled oligonucleotide probes. Radioactively labeled oligonucleotides are prepared by phosphorylalation of the 5′-prime end of two complementary oligonucleotides with T4 polynucleotede kinase. The complementary oligonucleotides are annealed and ligated to form concatemers. The double stranded concatemers are than radiolabled by for example nick transcription. Hybridization is normally performed at low stringency conditions using high oligonucleotide concentrations.

Oligonucleotide hybridization solution:

6×SSC

0.01 M sodium phosphate

1 mM EDTA (pH 8)

0.5% SDS

100 μg/ml denaturated salmon sperm DNA

0.1% nonfat dried milk

During hybridization temperature is lowered stepwise to 5-10° C. below the estimated oligonucleotid Tm.

Further details are described by Sambrook, J. et al. (1989), “Molecular Cloning: A Laboratory Manual”, Cold Spring Harbor Laboratory Press or Ausubel, F. M. et al. (1994) “Current Protocols in Molecular Biology”, John Wiley & Sons.

### Example 6

Identification of genes of interest by screening expression libraries with antibodies

cDNA sequences can be used to produce recombinant protein for example in E. coli (e. g. Qiagen QIAexpress pQE system). Recombinant proteins are than normally affinity purified via Ni—NTA affinity chromatoraphy (Qiagen). Recombinant proteins are than used to produce specific antibodies for example by using standard techniques for rabbit immunization. Antibodies are affinity purified using a Ni—NTA column saturated with the recombinant antigen as described by Gu et al., (1994)BioTechniques 17: 257-262. The antibody can than be used to screen expression cDNA libraries to identify homologous or heterologous genes via an immunological screening (Sambrook, J. et al. (1989), “Molecular Cloning: A Laboratory Manual”, Cold Spring Harbor Laboratory Press or Ausubel, F. M. et al. (1994) “Current Protocols in Molecular Biology”, John Wiley & Sons).

### Example 7

Northern-hybridization

For RNA hybridization, 20 mg of total RNA or 1 mg of poly-(A)⁺ RNA were separated by gel electrophoresis in 1.25% strength agarose gels using formaldehyde as described in Amasino (1986, Anal. Biochem. 152, 304), transferred by capillary attraction using 10×SSC to positively charged nylon membranes (Hybond N+, Amersham, Braunschweig), immobilized by UV light and prehybridized for 3 hours at 68° C. using hybridization buffer (10% dextran sulfate w/v, 1 M NaCl, 1% SDS, 100 mg of herring sperm DNA). The labeling of the DNA probe with the “Highprime DNA labeling kit” (Roche, Mannheim, Germany) was carried out during the prehybridization using alpha-³²P dCTP (Amersham, Braunschweig, germany). Hybridization was carried out after addition of the labeled DNA probe in the same buffer at 68° C. overnight. The washing steps were carried out twice for 15 min using 2×SSC and twice for 30 min using 1×SSC, 1% SDS at 68° C. The exposure of the sealed-in filters was carried out at −70° C. for a period of 1 to 4 d.

### Example 8

DNA Sequencing and Computational Functional Analysis

CDNA libraries libraries as described in Example 4 were used for DNA sequencing according to standard methods, in particular by the chain termination method using the ABI PRISM Big Dye Terminator Cycle Sequencing Ready Reaction Kit (Perkin-Ehner, Weiterstadt, germany). Random Sequencing was carried out subsequent to preparative plasmid recovery from cDNA libraries via in vivo mass excision and retransformation of DH10B on agar plates (material and protocol details from Stratagene, Amsterdam, Netherlands. Plasmid DNA was prepared from overnight grown E. coli cultures grown in Luria-Broth medium containing ampicillin (see Sambrook et al. (1989) (Cold Spring Harbor Laboratory Press: ISBN 0-87969-309-6)) on a Qiagene DNA preparation robot (Qiagen, Hilden) according to the manufacturers protocols. Sequencing primers with the following nucleotide sequences were used:

| | |
|---|---|
| 5′-CAGGAAACAGCTATGACC-3′ | (SEQ ID NO:179) |
| 5′-CTAAAGGGAACAAAAGCTG-3′ | (SEQ ID NO:180) |
| 5′-TGTAAAACGACGGCCAGT-3′ | (SEQ ID NO:181) |

### Example 9

Plasmids for plant transformation

For plant transformation binary vectors such as pBinAR can be used (Höfgen and Willmitzer (1990) Plant Science 66: 221-230). Construction of the binary vectors can be performed by ligation of the cDNA in sense or antisense orientation into the T-DNA. 5′ to the cDNA a plant promotor activates transcription of the cDNA. A polyadenylation sequence is located 3′ to the cDNA.

Tissue specific expression can be archived by using a tissue specific promotor. For example seed specific expression can be achived by cloning the napin or USP promotor 5′ to the cDNA. Also any other seed specific promotor element can be used. For constitutive expression within the whole plant the CaMV 35S promotor can be used. The expressed protein can be targeted to a cellular compartment using a signal peptide, for example for plastids, mitochondria or endoplasmatic reticulum (Kermode (1996) Crit. Rev. Plant Sci. 15: 285-423). The signal peptide is cloned 5′ in frame to the cDNA to achive subcellular localization of the fusion protein.

Nucleic acid molecules from Physomitrella patens are used for a direct gene knock-out by homologous recombination. Therefore Physcomitrella patens sequences are useful for functional genomic approaches. The technique is described by Strepp et al. (1998) Proc. Natl. Acad. Sci. USA 95: 4369-4373; Girke et al. (1998) Plant J. 15: 39-48; Hofmann et al. (1999) Molecular and General Genetics 261: 92-99.

### Example 10

Transformation of Agrobacterium

Agrobacterium mediated plant transformation can be performed using for example the GV3101(pMP90) (Koncz and Schell (1986) Mol. Gen. Genet. 204: 383-396) or LBA4404 (Clontech) Agrobacterium tumefaciens strain. Transformation can be performed by standard transformation techniques (Deblaere et al. (1984) Nucl. Acids 13: 4777-4788).

### Example 11

Plant transformation

Agrobacterium mediated plant transformation can be performed using standard transformation and regeneration techniques (Gelvin, S. B.; Schilperoort, R. A., “Plant Molecular Biology Manual”, 2nd Ed.—Dordrecht : Kluwer Academic Publ., 1995 and Glick, B. R., Thompson, J. E., “Methods in Plant Molecular Biology and Biotechnology”, Boca Raton: CRC Press, 1993.

For example, rapeseed can be transformed via cotyledon or hypocotyl transformation (Moloney et al.(1989) Plant Cell Report 8: 238-242; De Block et al. (1989) Plant Physiol. 91: 694-701). Use of antibiotica for Agrobacterium and plant selection depends on the binary vector and the agrobacterium strain used for transformation. Rapeseed selection is normally performed using kanamycin as selectable plant marker.

Agrobacterium mediated gene transfer to flax can be performed using for example a technique described by Mlynarova et al. (1994) Plant Cell Report 13: 282-285.

Transformation of soybean can be performed using for example a technique described in EP 0424 047, U.S. Pat. No. 322,783 (Pioneer Hi-Bred International) or in EP 0397 687, U.S. Pat. No. 5,376,543, U.S. Pat. No. 5,169,770 (University Toledo).

Plant transformation using particle bombardment, Polyethylene Glycol mediated DNA uptake or via the Silicon Carbide Fiber technique is for example described by Freeling and Walbot “The maize handbook” (1993) ISBN 3-540-97826-7, Springer Verlag New York).

### Example 12

In vivo Mutagenesis

In vivo mutagenesis of microorganisms can be performed by passage of plasmid (or other vector) DNA through E. coli or other microorganisms (e.g. Bacillus spp. or yeasts such as Saccharomyces cerevisiae) which are impaired in their capabilities to maintain the integrity of their genetic information. Typical mutator strains have mutations in the genes for the DNA repair system (e.g., mutBLS, mutD, mutT, etc.; for reference, see Rupp, W. D. (1996) DNA repair mechanisms, in: Escherichia coli and Salmonella, p. 2277-2294, ASM: Washington.) Such strains are well known to those skilled in the art. The use of such strains is illustrated, for example, in Greener, A. and Callahan, M. (1994) Strategies 7: 32-34. Transfer of mutated DNA molecules into plants is preferably done after selection and testing in microorganisms. Transgenic plants are generated according to various examples within the exemplification of this document.

### Example 13

DNA Transfer between Escherichia coli and Corynebacterium glutamicum

Several Corynebacterium and Brevibacterium species contain endogenous plasmids (as e.g., pHM1519 or pBL1) which replicate autonomously (for review see, e.g., Martin, J. F. et al. (1987) Biotechnology, 5:137-146). Shuttle vectors for Escherichia coli and Corynebacterium glutamicum can be readily constructed by using standard vectors for E. coli (Sambrook, J. et al. (1989), “Molecular Cloning: A Laboratory Manual”, Cold Spring Harbor Laboratory Press or Ausubel, F. M. et al. (1994) “Current Protocols in Molecular Biology”, John Wiley & Sons) to which a origin or replication for and a suitable marker from Corynebacterium glutamicum is added. Such origins of replication are preferably taken from endogenous plasmids isolated from Corynebacterium and Brevibacterium species. Of particular use as transformation markers for these species are genes for kanamycin resistance (such as those derived from the Tn5 or Tn903 transposons) or chloramphenicol (Winnacker, E. L. (1987) “From Genes to Clones—Introduction to Gene Technology, VCH, Weinheim). There are numerous examples in the literature of the construction of a wide variety of shuttle vectors which replicate in both E. coli and C. glutamicum, and which can be used for several purposes, including gene over-expression (for reference, see e.g., Yoshihama, M. et al. (1985) J. Bacteriol. 162:591-597, Martin J. F. et al. (1987) Biotechnology, 5:137-146 and Eikmanns, B. J. et al. (1991) Gene, 102:93-98). Using standard methods, it is possible to clone a gene of interest into one of the shuttle vectors described above and to introduce such a hybrid vectors into strains of Corynebacterium glutamicum. Transformation of C. glutamicum can be achieved by protoplast transformation (Kastsumata, R. et al. (1984) J. Bacteriol. 159306-311), electroporation (Liebl, E. et al. (1989) FEMS Microbiol. Letters, 53:399-303) and in cases where special vectors are used, also by conjugation (as described e.g. in Schäfer, A et al. (1990) J. Bacteriol. 172:1663-1666). It is also possible to transfer the shuttle vectors for C. glutamicum to E. coli by preparing plasmid DNA from C. glutamicum (using standard methods well-known in the art) and transforming it into E. coli. This transformation step can be performed using standard methods, but it is advantageous to use an Mcr-deficient E. coli strain, such as NM522 (Gough & Murray (1983) J. Mol. Biol. 166:1-19).

### Example 14

Assessment of the recombinant gene product in a transformed organism

The activity of a recombinant gene product in the transformed host organism can be measured on the transcriptional or/and on the translational level. A useful method to analyse the level of transcription of the transformed gene (an indicator of the amount of mRNA available for translation to the gene product) is to perform a Northern blot (for reference see, for example, Ausubel et al. (1988) Current Protocols in Molecular Biology, Wiley: New York), in which a primer designed to bind to the gene of interest is labeled with a detectable tag (usually radioactive or chemiluminescent), such that when the total RNA of a culture of the organism is extracted, run on gel, transferred to a stable matrix and incubated with this probe, the binding and quantity of binding of the probe indicates the presence and also the quantity of MnRNA for this gene. This information is evidence of the degree of transcription of the transformed gene. Total cellular RNA can be prepared from cells, tissues or organs by several methods, all well-known in the art, such as that described in Bormann, E. R. et al. (1992) Mol. Microbiol. 6: 317-326.

To assess the presence or relative quantity of protein translated from this mRNA, standard techniques, such as a Western blot, may be employed (see, for example, Ausubel et al. (1988) Current Protocols in Molecular Biology, Wiley: New York). In this process, total cellular proteins are extracted, separated by gel electrophoresis, transferred to a matrix such as nitrocellulose, and incubated with a probe, such as an antibody, which specifically binds to the desired protein. This probe is generally tagged with a chemiluminescent or calorimetric label which may be readily detected. The presence and quantity of label observed indicates the presence and quantity of the desired mutant protein present in the cell.

### Example 15

Growth of Genetically Modified Corynebacterium glutamicum—Media and Culture Conditions

Genetically modified Corynebacteria are cultured in synthetic or natural growth media. A number of different growth media for Corynebacteria are both well-known and readily available (Lieb et al. (1989) Appl. Microbiol. Biotechnol., 32:205-210; von der Osten et al. (1998) Biotechnology Letters, 11:11-16; Patent DE 4,120,867; Liebl (1992) “The Genus Corynebacterium, in: The Procaryotes, Volume II, Balows, A. et al., eds. Springer-Verlag). These media consist of one or more carbon sources, nitrogen sources, inorganic salts, vitamins and trace elements. Preferred carbon sources are sugars, such as mono-, di-, or polysaccharides. For example, glucose, fructose, mannose, galactose, ribose, sorbose, ribulose, lactose, maltose, sucrose, raffinose, starch or cellulose serve as very good carbon sources. It is also possible to supply sugar to the media via complex compounds such as molasses or other by-products from sugar refinement. It can also be advantageous to supply mixtures of different carbon sources. Other possible carbon sources are alcohols and organic acids, such as methanol, ethanol, acetic acid or lactic acid. Nitrogen sources are usually organic or inorganic nitrogen compounds, or materials which contain these compounds. Exemplary nitrogen sources include ammonia gas or ammonia salts, such as NH₄Cl or (NH₄)₂SO₄, NH₄OH, nitrates, urea, amino acids or complex nitrogen sources like corn steep liquor, soy bean flour, soy bean protein, yeast extract, meat extract and others.

Inorganic salt compounds which may be included in the media include the chloride-, phosphorous- or sulfate- salts of calcium, magnesium, sodium, cobalt, molybdenum, potassium, manganese, zinc, copper and iron. Chelating compounds can be added to the medium to keep the metal ions in solution. Particularly useful chelating compounds include dihydroxyphenols, like catechol or protocatechuate, or organic acids, such as citric acid. It is typical for the media to also contain other growth factors, such as vitamins or growth promoters, examples of which include biotin, riboflavin, thiamin, folic acid, nicotinic acid, pantothenate and pyridoxin. Growth factors and salts frequently originate from complex media components such as yeast extract, molasses, corn steep liquor and others. The exact composition of the media compounds depends strongly on the immediate experiment and is individually decided for each specific case. Information about media optimization is available in the textbook “Applied Microbiol. Physiology, A Practical Approach (eds. P. M. Rhodes, P. F. Stanbury, IRL Press (1997) pp. 53-73, ISBN 0 19 963577 3). It is also possible to select growth media from commercial suppliers, like standard 1 (Merck) or BHI (grain heart infusion, DIFC) or others.

All medium components are sterilized, either by heat (20 minutes at 1.5 bar and 121° C.) or by sterile filtration. The components can either be sterilized together or, if necessary, separately. All media components can be present at the beginning of growth, or they can optionally be added continuously or batchwise.

Culture conditions are defined separately for each experiment. The temperature should be in a range between 15° C. and 45° C. The temperature can be kept constant or can be altered during the experiment. The pH of the medium should be in the range of 5 to 8.5, preferably around 7.0, and can be maintained by the addition of buffers to the media. An exemplary buffer for this purpose is a potassium phosphate buffer. Synthetic buffers such as MOPS, AEPES, ACES and others can alternatively or simultaneously be used. It is also possible to maintain a constant culture pH through the addition of NaOH or NH₄OH during growth. If complex medium components such as yeast extract are utilized, the necessity for additional buffers may be reduced, due to the fact that many complex compounds have high buffer capacities. If a fermentor is utilized for culturing the micro-organisms, the pH can also be controlled using gaseous ammonia.

The incubation time is usually in a range from several hours to several days. This time is selected in order to permit the maximal amount of product to accumulate in the broth. The disclosed growth experiments can be carried out in a variety of vessels, such as microtiter plates, glass tubes, glass flasks or glass or metal fermentors of different sizes. For screening a large number of clones, the microorganisms should be cultured in microtiter plates, glass tubes or shake flasks, either with or without baffles. Preferably 100 ml shake flasks are used, filled with 10% (by volume) of the required growth medium. The flasks should be shaken on a rotary shaker (amplitude 25 mm) using a speed-range of 100-300 rpm. Evaporation losses can be diminished by the maintenance of a humid atmosphere; alternatively, a mathematical correction for evaporation losses should be performed.

If genetically modified clones are tested, an unmodified control clone or a control clone containing the basic plasmid without any insert should also be tested. The medium is inoculated to an OD₆₀₀ of 0.5-1.5 using cells grown on agar plates, such as CM plates (10 g/l glucose, 2,5 g/l NaCl, 2 g/l urea, 10 g/l polypeptone, 5 g/l yeast extract, 5 g/l meat extract, 22 g/l NaCl, 2 g/l urea, 10 g/l polypeptone, 5 g/l yeast extract, 5 g/l meat extract, 22 g/l agar, pH 6.8 with 2M NaOH) that had been incubated at 30° C. Inoculation of the media is accomplished by either introduction of a saline suspension of C. glutamicum cells from CM plates or addition of a liquid preculture of this bacterium.

### Example 16

In vitro Analysis of the Function of Physcomitrella Genes in Transgenic Organisms

The determination of activities and kinetic parameters of enzymes is well established in the art. Experiments to determine the activity of any given altered enzyme must be tailored to the specific activity of the wild-type enzyme, which is well within the ability of one skilled in the art. Overviews about enzymes in general, as well as specific details concerning structure, kinetics, principles, methods, applications and examples for the determination of many enzyme activities may be found, for example, in the following references: Dixon, M., and Webb, E. C., (1979) Enzymes. Longmans: London; Fersht, (1985) Enzyme Structure and Mechanism. Freeman: New York; Walsh, (1979) Enzymatic Reaction Mechanisms. Freeman: San Francisco; Price, N. C., Stevens, L. (1982) Fundamentals of Enzymology. Oxford Univ. Press: Oxford; Boyer, P. D., ed. (1983) The Enzymes, 3^{rd} ed. Academic Press: New York; Bisswanger, H., (1994) Enzymiinetik, 2^{nd} ed. VCH: Weinheim (ISBN 3527300325); Bergmeyer, H. U., Bergmeyer, J., Grall, M., eds. (1983-1986) Methods of Enzymatic Analysis, 3^{rd} ed., vol. I-XHI, Verlag Chemie: Weinheim; and Ullmann's Encyclopedia of Industrial Chemistry (1987) vol. A9, “Enzymes”. VCH: Weinheim, p. 352-363.

The activity of proteins which bind to DNA can be measured by several well-established methods, such as DNA band-shift assays (also called gel retardation assays; described in Mikami, K., Takase, H. and Iwabuchi, M. (1995) Gel mobility shift assay, in ‘Plant Molecular Biology Manual’, Second edition, Gelvin, S. B. and Schilperoort, R. A. (eds.), Kluwer Academic Publishers, section I1, pp. 1-14). The effect of such proteins on the expression of other molecules can be measured using reporter gene assays (such as that described in Kolmar, H. et al. (1995) EMBO J. 14: 3895-3904 and references cited therein). Reporter gene test systems are well known and established for applications in both pro- and eukaryotic cells, using enzymes such as beta-galactosidase, green fluorescent protein, and several others.

The determination of activity of membrane-transport proteins can be performed according to techniques such as those described in Gennis, R. B. (1989) “Pores, Channels and Transporters”, in Biomembranes, Molecular Structure and Function, Springer: Heidelberg, p. 85-137; 199-234; and 270-322.

### Example 17

Analysis of Impact of Recombinant Proteins on the Production of the Desired Product

The effect of the genetic modification in higher plants, C. glutamicum, other bacteria, fungi or algae on production of a desired compound (such as carbohydrates) can be assessed by growing the modified microorganism or plant under suitable conditions (such as those described above) and analyzing the medium and/or the cellular component for increased production of the desired product (i.e., carbohydrates). Such analysis techniques are well known to one skilled in the art, and include spectroscopy, thin layer chromatography, staining methods of various kinds, enzymatic and microbiological methods, and analytical chromatography such as high performance liquid chromatography (see, for example, Ullman, Encyclopedia of Industrial Chemistry, vol. A2, p. 89-90 and p. 443-613, VCH: Weinheim (1985); Fallon, A. et al., (1987) “Applications of HPLC in Biochemistry” in: Laboratory Techniques in Biochemistry and Molecular Biology, vol. 17; Rehm et al. (1993) Biotechnology, vol. 3, Chapter III: “Product recovery and purification”, page 469-714, VCH: Weinheim; Belter, P. A. et al. (1988) Bioseparations: downstream processing for biotechnology, John Wiley and Sons; Kennedy, J. F. and Cabral, J. M. S. (1992) Recovery processes for biological materials, John Wiley and Sons; Shaeiwitz, J. A. and Henry, J. D. (1988) Biochemical separations, in: Ulmann's Encyclopedia of Industrial Chemistry, vol. B3, Chapter 11, page 1-27, VCH: Weinheim; and Dechow, F. J. (1989) Separation and purification techniques in biotechnology, Noyes Publications.)

In addition to the measurement of the final product of plant growth or fermentation, it is also possible to analyze other components of the metabolic pathways utilized for the production of the desired compound, such as intermediates and side-products, to determine the overall efficiency of production of the compound. Analysis methods include measurements of nutrient levels in the medium (e.g., sugars, hydrocarbons, nitrogen sources, phosphate, and other ions), measurements of biomass composition and growth, analysis of the production of common metabolites of biosynthetic pathways, and measurement of gasses produced during fermentation. Standard methods for these measurements are outlined in Applied Microbial Physiology, A Practical Approach, P. M. Rhodes and P. F. Stanbury, eds., IRL Press, p. 103-129; 131-163; and 165-192 (ISBN: 0199635773) and references cited therein.

One example for the analysis of final products and its constituents is the analysis of starch and starch compounds:

Starch is extracted from plant material e.g. as described by Zeeman, S. C., Northrop, F., Smith, A. M. and ap Rees, T. (1998) Plant J. 15: 357-365 or by Edwards, A., Marshall J., Sidebottom, D., Visser, R. G. F., Smith, A. M., Martin, C. (1995). This involves grinding up plant samples in a mechanical blender with 50 mM Tris-HCl (pH 7.0), 1 mM EDTA, 1 mM DTT, 10 mg 1-1 Na-metabisulfate before allowing the starch to sediment at 4° C. The starch is resuspended in buffer and filtered through two layers of Miracloth (Calbiochem, La Jolla, Calif., USA) before being centrifuged at 2000×g and 4° C. for 10 min. This step is repeated four more times. The starch is washed three times with cooled acetone (−20° C.) before being allowed to air dry, and is then stored at −20° C. before use. The amylose content of starch can be measured e.g. by a spectralphotometric method that is described in Hovenkamp-Hermelink J. H. M., De Vries, J. N., Adamse, P., Jacobsen, E., Witholt, B., Feenstra, W. J. (1988) Potato Research 31: 241-246. Amylopectin can be isolated from purified starch e.g. by selectively precipitating the amylose fraction using the chemical thymol, according to Tomlinson, K. L., Lloyd, J. R., Smith, A. M. (1997) Plant J. 11: 31-43. To study the constituent chains of the amylopectin, the purified amylopectin can be digested with Pseudomonas isoamylase as described in Lloyd, J. R., Springer, F., Buleon, A., Müller-Röber, B., Willmitzer, L. and Kossmann, J. (1999). Size exclusion HPLC can be used for the analysis of the amylose/amylopectin ratio. HPAEC is a preferred method for the determination of the amylopectin chain length (see Zeeman, S. C., Umemoto, T., Lue, W. -L., Pui, A. -Y., Martin, C., Smith, A. M. and Chen, J. (1998) Plant Cell 10: 1699-1711.

A protocol for the determination of starch contents and glucose-6-phosphate contents of the starch is described in Nielsen, T. H., Wischmann, B., Enevoldsen, K., Moller, B. L. (1994) Plant Physiol. 105: 111-117. The starch is digested to glucose either by using amyloglucosidase or by hydrolysis in 0.7 N HCl at 95° C. The glucose as well as the glucose-6-phosphate content can be determined via enzymatic assays.

Another example for the analysis of final products and its constituents is the analysis of cell wall carbohydrates:

Cellulose can be quantified e.g. as described by Updegraff, D. M. (1969) Analytical Biochem. 32: 420-424. This method involves the extraction of cellulose from organic material with acetic/nitric acid and the hydrolysis with concentrated sulfuric acid. The resulting glucose is then quantified via the spectralphotometrical anthron assay. Moreover cellulose microfibrills can be detected by staining with calcofluor white (see e.g. Haigler, C. H., Brown, R. M. Jr., Benziman, M. (1980) Science 210: 903-906. The monosaccharide composition of the matrix polysaccharides (i.e. hemicelluloses and pectins) can be analysed as described in Keller, R., Springer, F., Renz, A. and Kossmann, J. (1999). This method involves an phenol/acetic acid/chloroform extraction and the hydrolysis of non-cellulosic polysaccharides in 1 M TFA. The resulting monosaccharides can be separated by anion-exchange HPLC and are detected by pulsed amperometry after a post column derivatization step. Alternatively the monosaccharide composition can be analysed via gas-liquid chromatography of alditol acetates as described by Reiter, W. D., Chapple, C. C. S. and Somerville, C. R. (1993) Science 261: 1032-1035 or by other chromatographic methods.

Another suitable method for the analysis of cell wall carbohydrates is given by immunolocalisation using antibodies raised against specific cell wall compounds. E.g. JIM 5 and JIM 7 monoclonal antibodies can be used for the detection of unesterified and esterified pectins, respectively (see Dolan, L., Linstead, P. and Roberts, K. (1997) J. Exp. Bot. 308: 713-720, and Steele, N. M., McCann, M. C. and Roberts, K. (1997) Plant Physiol. 114: 373-381).

A method for the quantification of uronic acids in pectins is described e.g. in Blumenkrantz, N. and Asboe-Hansen, G. (1973) Anal. Biochem. 54: 484-489.

The impact of an altered cell wall polysaccharide composition on the mechanical properties of a plant can be analysed by testing the physical stability of stem segments as described in Turner, S. R. and Somerville, C. R. (1997) Plant Cell 9: 689-701.

Another example for the analysis of final products and its constituents is the analysis of soluble sugars:

Glucose, fructose and sucrose can be extracted with ethanol and measured using spectralphotometrical assays as described by Stitt, M., Lilley, McC., Gerhardt, R., Heldt, H. W. (1989) In: Methods in Enzymology Vol. 174, Fleischer, S., Fleischer, R. (eds.), Academic Press Ltd., London, UK, pp. 518-552). In the same reference protocols for the extraction and measurement of hexose-phosphates, fructose-1,6-bisphosphate and triose-phosphates are described. Sucrose can also be quantified by the anthron test as described in Geigenberger, P., Hajirezaei, M., Geiger, M., Deiting, U., Sonnewald, U. and Stitt, M. (1998) Planta 205: 428-437 and in the references therein.

The extraction and analysis of trehalose and its metabolite trehalose-6-phosphate from plant materails are described in Goddijn, O. J. M. et al. (1997) Plant Physiol. 113: 181-190 and in Drennan, P. M. et al. (1993) J. Plant Physiol. 142: 493-496.

The trisaccharide raffinose can be analysed by TLC, GC or other chromatographic methods as described in Muzquiz, M. Burbano, C., Pedrosa, M. M., Folkman, W. and Gulewicz, K. (1999) Industrial Crops and Products 9: 183-188 and references cited therein.

### Example 18

Purification of the Desired Product from Transformed Organisms

Recovery of the desired product from plant materials or fungi, algae and bacteria like Acetobacter xylinum or C. glutamicum cells or supernatant of the above-described culture can be performed by various methods well known in the art. If the desired product is not secreted from the cells, the cells, can be harvested from the culture by low-speed centrifugation, the cells can be lysed by standard techniques, such as mechanical force or sonication. Organs of plants can be separated mechanically from other tissues or organs. Following homogenization, cellular debris is removed by centrifugation, and the supernatant fraction containing the soluble proteins is retained for further purification of the desired compound. If the product is secreted from desired cells, then the cells are removed from the culture by low-speed centrifugation, and the supernatant fraction is retained for further purification.

The supernatant fraction from either purification method is subjected to chromatography with a suitable resin, in which the desired molecule is either retained on a chromatography resin while many of the impurities in the sample are not, or where the impurities are retained by the resin while the sample is not. Such chromatography steps may be repeated as necessary, using the same or different chromatography resins. One skilled in the art would be well-versed in the selection of appropriate chromatography resins and in their most efficacious application for a particular molecule to be purified. The purified product may be concentrated by filtration or ultrafiltration, and stored at a temperature at which the stability of the product is maximized.

There are a wide array of purification methods known to the art and the preceding method of purification is not meant to be limiting. Such purification techniques are described, for example, in Bailey, J. E. & Ollis, D. F. Biochemical Engineering Fundamentals, McGraw-Hill: New York (1986).

The identity and purity of the isolated compounds may be assessed by techniques standard in the art. These include high-performance liquid chromatography (HPLC), spectroscopic methods, staining methods, thin layer chromatography, NIRS, enzymatic assay, or microbiologically. Such analysis methods are reviewed in: Patek et al. (1994) Appl. Environ. Microbiol. 60: 133-140; Malakhova et al. (1996) Biotekhnologiya 11: 27-32; and Schmidt et al. (1998) Bioprocess Engineer. 19: 67-70. Ulmann's Encyclopedia of Industrial Chemistry, (1996) vol. A27, VCH: Weinheim, p. 89-90, p. 521-540, p. 540-547, p. 559-566, 575-581 and p. 581-587; Michal, G. (1999) Biochemical Pathways: An Atlas of Biochemistry and Molecular Biology, John Wiley and Sons; Fallon, A. et al. (1987) Applications of BPLC in Biochemistry in: Laboratory Techniques in Biochemistry and Molecular Biology, vol. 17.

One example for the preparation of desired products from plants is the isolation of starch. Various wet-milling and other starch extraction techniques are described in the literature, depending on the crop plant and on the industrial application (e.g. see in Ellis, R. P. et al. (1998) Journal of the Science of Food and Agriculture 77: 289-311; Singh, S. K. et al. (1997) Cereal Chemistry 74 and references cited therin).

Equivalents

Those skilled in the art will recognize, or will be able to ascertain using no more than routine experimentation, many equivalents to the specific embodiments of the invention described herein. Such equivalents are intended to be encompassed by the following claims.

Legends to the Figures:

Table 1: Enzymes involved in production of carbohydrates, the accession/entry number of the corresponding partial nucleic acid molecules, the entry number of longest clones corresponding to partial nucleic acid molecules and the position of open reading frame.

Appendix A: Nucleic acid sequences encoding for CMR (Carbohydrate Metabolism Related) polypeptides (SEQ ID NO:1 to SEQ ID NO:177, odd integers)

Appendix B: CMR polypeptide sequences (SEQ ID NO:2 to SEQ ID NO:178, even integers)

TABLE 1

| | | Start of open | Stop of open |
|---|---|---|---|
| Enzyme encoded | Acc. no./Entry no. | reading frame | reading frame |
| Hemicellulose metabolism | | | |
| UDP-glucose dehydrogenase | 18_ck32_c09fwd | 1-3 | 547-549 |
| | (SEQ ID NO: 1, SEQ ID NO:2) | | |
| UDP-N-acetylglucosamine O-acyltransferase- | 21_ppprot1_047_d02 | 1-3 | 544-546 |
| like protein | (SEQ ID NO: 3, SEQ ID NO: 4) | | |
| GDP-D-mannose dehydratase | 91_ppprot1_055_h04 | 2-4 | 161-163 |
| | (SEQ ID NO: 5, SEQ ID NO: 6) | | |
| GDP-D-mannose dehydratase | 51_ppprot1_056_a05 | 3-5 | 282-284 |
| | (SEQ ID NO: 7, SEQ ID NO: 8) | | |
| GPD-D-mannose dehydratase | 05_ppprot1_090_a03 | 1-3 | 139-141 |
| | (SEQ ID NO: 9, SEQ ID NO: 10) | | |
| GDP-D-mannose dehydratase | 15_ppprot1_080_c02 | 3-5 | 192-194 |
| | (SEQ ID NO: 11, SEQ ID NO: 12) | | |
| GDP-D-mannose dehydratase | 80_ppprot1_092_f10 | 1-3 | 316-318 |
| | (SEQ ID NO: 13, SEQ ID NO: 14) | | |
| GDP-4-keto-6-deoxy-D-mannose 3,5- | 20_ppprot1_064_d07 | 2-4 | 485-487 |
| epimerase-4-reductase | (SEQ ID NO: 15, SEQ ID NO: 16) | | |
| Xyloglucan endotransglycosylase | 41_ppprot_069_g03 | 2-4 | 338-340 |
| | (SEQ ID NO: 17, SEQ ID NO: 18) | | |
| Xyloglucan endotransglycosylase | 48_ck10_h09fwd | 104-106 | 500-502 |
| | (SEQ ID NO: 19, SEQ ID NO: 20) | | |
| Xyloglucan endotransglycosylase | 18_ppprot1_055_c09 | 2-4 | 392-394 |
| | (SEQ ID NO: 21, SEQ ID NO: 22) | | |
| Xyloglucan endotransglycosylase | 90_ppprot1_056_g12 | 3-5 | 429-431 |
| | (SEQ ID NO: 23, SEQ ID NO: 24) | | |
| Endoxyloglucan transferase | 37_ppprot1_051_g01 | 237-239 | 618-620 |
| | (SEQ ID NO: 25, SEQ ID NO: 26) | | |
| Endoxyloglucan transferase | 35_mm14_f03rev | 102-104 | 567-569 |
| | (SEQ ID NO: 27, SEQ ID NO: 28) | | |
| Endoxyloglucan transferase | 96_ppprot1_081_h12 | 1-3 | 430-432 |
| | (SEQ ID NO: 29, SEQ ID NO: 30) | | |
| Beta-1,3-glucanase | 96_ck7_h12fwd | 2-4 | 515-517 |
| | (SEQ ID NO: 31, SEQ ID NO: 32) | | |
| Beta-D-glucan exohydrolase | 37_mm21_g01rev | 3-5 | 513-515 |
| | (SEQ ID NO: 33, SEQ ID NO: 34) | | |
| Pectine metabolism | | | |
| polygalacturonase | 10_ppprot1_085_b08 | 1-3 | 238-240 |
| | (SEQ ID NO: 35, SEQ ID NO: 36) | | |
| Cellulose metabolism | | | |
| Cellulose synthase catalytic subunit | 16_mm6 | 2-4 | 494-496 |
| | (SEQ ID NO: 37, SEQ ID NO: 38) | | |
| Cellulose synthase catalytic subunit | 83_mm10_f06rev | 3-5 | 477-479 |
| | (SEQ ID NO: 39, SEQ ID NO: 40) | | |
| Cellulose synthase catalytic subunit | 09_mm10_b02rev | 1-3 | 343-345 |
| | (SEQ ID NO: 41, SEQ ID NO: 42) | | |
| Beta-glucosidase | 67_mm22_d04rev | 3-5 | 519-521 |
| | (SEQ ID NO: 43, SEQ ID NO: 44) | | |
| Sugar metabolism | | | |
| Trehalose-6-phosphate phosphatase | 73_ck12_e04fwd | 1-3 | 304-306 |
| | (SEQ ID NO: 45, SEQ ID NO: 46) | | |
| Trehalose-6-phosphate synthase | 63_ck23_c05fwd | 1-3 | 517-519 |
| | (SEQ ID NO: 47, SEQ ID NO: 48) | | |
| Trehalose-6-phosphate synthase | 80_ck30_f10fwd | 3-5 | 537-539 |
| | (SEQ ID NO: 49, SEQ ID NO: 50) | | |
| Plastidic triosephosphate isomerase | 46_ck2_h08fwd (SEQ | 187-189 | 469-471 |
| | ID NO: 51, SEQ ID NO: 52) | | |
| Plastidic triosephosphate isomerase | 83_bd06_f06rev (SEQ | 1-3 | 364-366 |
| | ID NO: 53, SEQ ID NO: 54) | | |
| Cytosolic phosphoglucomutase | 19_ck1_d01fwd (SEQ | 1-3 | 331-333 |
| | ID NO: 55, SEQ ID NO: 56) | | |
| Fructokinase | 11_ck_19_b03 (SEQ | 1-3 | 208-210 |
| | ID NO: 57, SEQ ID NO: 58) | | |
| Hexokinase | 56_ppprot1_061_b10 | 2-4 | 392-394 |
| | (SEQ ID NO: 59, SEQ ID NO: 60) | | |
| UDP-glucose pyrophosphorylase | 18_ppprot1_064_c09 | 1-3 | 346-348 |
| | (SEQ ID NO: 61, SEQ ID NO: 62) | | |
| Sucrose synthase | 76_ck27_e11fwd | 2-4 | 494-496 |
| | (SEQ ID NO: 63, SEQ ID NO: 64) | | |
| Invertase | 71_ck18_d06fwd | 1-3 | 373-375 |
| | (SEQ ID NO: 65, SEQ ID NO: 66) | | |
| Invertase | 94_ck14_h11fwd | 1-3 | 295-297 |
| | (SEQ ID NO: 67, SEQ ID NO: 68) | | |
| Sucrolytic enzyme | 25_bd07_e01rev | 3-5 | 279-281 |
| | (SEQ ID NO: 69, SEQ ID NO: 70) | | |
| Sucrose phophate synthase | 66_ppprot1_075_c12 | 1-3 | 412-414 |
| | (SEQ ID NO: 71, SEQ ID NO: 72) | | |
| Glucose-6-phosphate isomerase | 50_mm15_a10rev | 1-3 | 574-576 |
| | (SEQ ID NO: 73, SEQ ID NO: 74) | | |
| Phosphoenolpyruvate carboxylase | 70_mm10_d11rev | 3-5 | 474-476 |
| | (SEQ ID NO: 75, SEQ ID NO: 76) | | |
| Pyruvate dehydrogenase | 12_ck22_b09fwd | 3-5 | 441-443 |
| | (SEQ ID NO: 77, SEQ ID NO: 78) | | |
| Citrate synthetase | 37_mm3_g01rev | 88-90 | 486-489 |
| | (SEQ ID NO: 79, SEQ ID NO: 80) | | |
| Ribokinase | 70_ppprot1_069_d11 | 107-109 | 623-625 |
| | (SEQ ID NO: 81, SEQ ID NO: 82) | | |
| Cytosolic pyruvate kinase | 96_ck20_h12fwd | 2-4 | 248-250 |
| | (SEQ ID NO: 83, SEQ ID NO: 84) | | |
| Carboxyphosphoenolpyruvate mutase | 88_mm13_g11rev | 9-11 | 384-386 |
| | (SEQ ID NO: 85, SEQ ID NO: 86) | | |
| Phosphoribulokinase | 18_ck25_c09fwd | 3-5 | 429-431 |
| | (SEQ ID NO: 87, SEQ ID NO: 88) | | |
| 3-Phosphoglycerate dehydrogenase | 83_ck30_f06fwd | 2-4 | 518-520 |
| | (SEQ ID NO: 89, SEQ ID NO: 90) | | |
| Cytosolic phosphoglycerate kinase | 63_ck7_c05fwd | 2-4 | 386-388 |
| | (SEQ ID NO: 91, SEQ ID NO: 92) | | |
| Plastidial phosphoglycerate kinase | 18_ck24_c09fwd | 92-94 | 476-478 |
| | (SEQ ID NO: 93, SEQ ID NO: 94) | | |
| Chloroplastic fructose bisphosphate | 18_ck26_c09fwd | 200-202 | 440-442 |
| aldolase | (SEQ ID NO: 95, SEQ ID NO: 96) | | |
| Chloroplastic fructose bisphosphate | 60_ppgam17_b12 | 75-77 | 402-404 |
| aldolase | (SEQ ID NO: 97, SEQ ID NO: 98) | | |
| Plastidial SBPase | 50_ck19_a10fwd | 2-4 | 167-169 |
| | (SEQ ID NO: 99, SEQ ID NO: 100) | | |
| Plastidial FBPase | 35_ck11_f03fwd | 65-67 | 572-574 |
| | (SEQ ID NO: 101, SEQ ID NO: 102) | | |
| Fructose-6-phosphate 2-kinase/ | 20_ppprot1_083_d07 | 3-5 | 243-245 |
| fructose-2,6-bisphosphatase | (SEQ ID NO: 103, SEQ ID NO: 104) | | |
| 3-Deoxy-D-arabino-heptulosonate 7- | 14_ck4_c07fwd | 3-5 | 181-182 |
| phosphate synthase (shkB) | (SEQ ID NO: 105, SEQ ID NO: 106) | | |
| 3-Deoxy-d-manno-octulosonic acid 8- | 89_ck12_g06fwd | 2-4 | 422-424 |
| phosphate synthase | (SEQ ID NO: 107, SEQ ID NO: 108) | | |
| Ribulose-phosphate-3-epimerase | 55_bd01_b04rev | 76-78 | 340-342 |
| (pentose-5-phosphate-3-epimerase) | (SEQ ID NO: 109, SEQ ID NO: 110) | | |
| Cytosolic glucose-6-phosphate | 50_mm15_a10rev | 1-3 | 574-576 |
| isomerase | (SEQ ID NO: 111, SEQ ID NO: 112) | | |
| Ribose-5-P isomerase | 86_ck23g_g10fwd | 239-241 | 452-454 |
| | (SEQ ID NO: 113, SEQ ID NO: 114) | | |
| Lysosomal alpha-mannosidase | 22_ppgam15_d08 | 1-3 | 334-336 |
| | (SEQ ID NO: 115, SEQ ID NO: 116) | | |
| Transporter | | | |
| Triosephosphate transporter | 70_ck11_d11fwd | 2-4 | 464-466 |
| | (SEQ ID NO: 117, SEQ ID NO: 118) | | |
| ADP/ATP carrier protein | 29_ck12_e03fwd | 3-5 | 282-284 |
| | (SEQ ID NO: 119, SEQ ID NO: 120) | | |
| Sucrose transporter | 07_ppprot1_057_b01 | 3-5 | 159-161 |
| | (SEQ ID NO: 121, SEQ ID NO: 122) | | |
| Sucrose transporter | 25_ppprot1_057_e01 | 1-3 | 208-210 |
| | (SEQ ID NO: 123, SEQ ID NO: 124) | | |
| Sugar transporter | 48_ck24_h09fwd | 1-3 | 517-519 |
| | (SEQ ID NO: 125, SEQ ID NO: 126) | | |
| Starch catabolism | | | |
| Alpha-glucosidase | 41_ppprot1_105_g03 | 1-3 | 463-465 |
| | (SEQ ID NO: 127, SEQ ID NO: 128) | | |
| Alpha-glucosidase | 44_ppprot1_075_h07 | 1-3 | 595-597 |
| | (SEQ ID NO: 129, SEQ ID NO: 130) | | |
| Alpha-glucosidase | 63_ppprot1_60 | 3-5 | 705-707 |
| | (SEQ ID NO: 131, SEQ ID NO: 132) | | |
| Alpha-glucosidase | 74_ck13_e10fwd | 2-4 | 563-565 |
| | (SEQ ID NO: 133, SEQ ID NO: 134) | | |
| Alpha-amylase | 03_ppprot1_056_a02 | 1-3 | 316-318 |
| | (SEQ ID NO: 135, SEQ ID NO: 136) | | |
| Alpha-amylase | 50_ck1_a10fwd | 2-4 | 599-601 |
| | (SEQ ID NO: 137, SEQ ID NO: 138) | | |
| Beta-amylase | 25_ppprot1_104_e01 | 2-4 | 548-550 |
| | (SEQ ID NO: 139, SEQ ID NO: 140) | | |
| Starch anabolism | | | |
| ADP glucose pyrophosphorylase | 53_ppprot1_074_a06 | 3-5 | 213-215 |
| large subunit | (SEQ ID NO: 141, SEQ ID NO: 142) | | |

Additional clones; full length

| Function/Amino acid | Clone entry no. | Clone entry no. of | Start of open | Stop- |
|---|---|---|---|---|
| metabolism | of longest clone | corresponding partial clone | reading frame | codon |
| UDP-N-acetylglucosamine | s_pp001047038r | 21_ppprot1_047_d02 | 2-4 | 551-553 |
| O-acyltransferase- | (SEQ ID NO: 143, SEQ ID NO: 144) | (SEQ ID NO: 3, SEQ ID NO: 4) | | |
| like protein | | | | |
| GDP-D-mannose | c_pp030002055r | 91_ppprot1_055_h04 | 224-226 | 1268-1270 |
| dehydratase | (SEQ ID NO: 145, SEQ ID NO: 146) | (SEQ ID NO: 5, SEQ ID NO: 6) | | |
| GDP-4-keto-6-deoxy- | c_pp001064043r | 20_ppprot1_064_d07 | 347-349 | 1274-1276 |
| D-mannose 3,5- | (SEQ ID NO: 147, SEQ ID NO: 148) | (SEQ ID NO: 15, SEQ ID NO: 16) | | |
| epimerase-4-reductase | | | | |
| Endoxyloglucan | c_pp032009028r | 37_ppprot1_051_g01 | 322-324 | 919-921 |
| transferase | (SEQ ID NO: 149, SEQ ID NO: 150) | (SEQ ID NO: 25, SEQ ID NO: 26) | | |
| Endoxyloglucan | c_pp004089354r | 35_mm14_f03rev | 268-270 | 1126-1128 |
| transferase | (SEQ ID NO: 151, SEQ ID NO: 152) | (SEQ ID NO: 27, SEQ ID NO: 28) | | |
| Cellulose synthase | s_pp002010066r | 83_mm10_f06rev | 1-3 | 499-501 |
| catalytic subunit | (SEQ ID NO: 153, SEQ ID NO: 154) | (SEQ ID NO: 39, SEQ ID NO: 40) | | |
| Plastidic | c_pp001002092f | 46_ck2_h08fwd | 187-189 | 1165-1167 |
| triosephosphate isomerase | (SEQ ID NO: 155, SEQ ID NO: 156) | (SEQ ID NO: 51, SEQ ID NO: 52) | | |
| Plastidic | s_pp013006066r | 83_bd06_f06rev | 3-5 | 915-917 |
| triosephosphate isomerase | (SEQ ID NO: 157, SEQ ID NO: 158) | (SEQ ID NO: 39, SEQ ID NO: 40) | | |
| Fructokinase | c_pp004048178r | 11_ck_19_b03 | 2-4 | 776-778 |
| | (SEQ ID NO: 159, SEQ ID NO: 160) | (SEQ ID NO: 57, SEQ ID NO: 58) | | |
| Invertase | c_pp001074086r | 71_ck18_d06fwd | 1-3 | 796-798 |
| | (SEQ ID NO: 161, SEQ ID NO: 162) | (SEQ ID NO: 65, SEQ ID NO: 66) | | |
| Sucrolytic enzyme | c_pp004102322r | 25_bd07_e01rev | 482-484 | 1388-1390 |
| | (SEQ ID NO: 163, SEQ ID NO: 164) | (SEQ ID NO: 69, SEQ ID NO: 70) | | |
| Cytosolic | c_pp004089380r | 63_ck7_c05fwd | 135-137 | 1557-1559 |
| phosphoglycerate kinase | (SEQ ID NO: 165, SEQ ID NO: 166) | (SEQ ID NO: 91, SEQ ID NO: 92) | | |
| Triosephosphate | c_pp004044298r | 70_ck11_d11fwd | 81-83 | 1158-1160 |
| transporter | (SEQ ID NO: 167, SEQ ID NO: 168) | (SEQ ID NO: 117, SEQ ID NO: 118) | | |
| ADP/ATP carrier | c_pp004075307r | 29_ck12_e03fwd | 82-84 | 1237-1239 |
| protein | (SEQ ID NO: 169, SEQ ID NO: 170) | (SEQ ID NO: 119, SEQ ID NO: 120) | | |
| Sugar transporter | s_pp001024093f | 48_ck24_h09fwd | 1-3 | 1438-1440 |
| | (SEQ ID NO: 171, SEQ ID NO: 172) | (SEQ ID NO: 125, SEQ ID NO: 126) | | |
| Alpha-amylase | c_pp010010057r | 50_ck1_a10fwd | 1-3 | 1288-1290 |
| | (SEQ ID NO: 173, SEQ ID NO: 174) | (SEQ ID NO: 137, SEQ ID NO: 138) | | |
| Beta-amylase | c_pp004072377r | 25_ppprot1_104_e01 | 168-170 | 1626-1628 |
| | (SEQ ID NO: 175, SEQ ID NO: 176) | (SEQ ID NO: 139, SEQ ID NO: 140) | | |
| ADP glucose pyro- | c_pp001109095r | 53_ppprot1_074_a06 | 736-738 | 2053-2055 |
| phosphorylase large subunit | (SEQ ID NO: 177, SEQ ID NO: 178) | (SEQ ID NO: 141, SEQ ID NO: 142) | | |

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

1. An isolated nucleic acid molecule from a moss encoding a Carbohydrate Metabolism Related Protein (CMRP), or a portion thereof.

2. The isolated nucleic acid molecule of claim 1 wherein the moss is selected from Physcomitrella patens or Ceratodon purpureus.

3. The isolated nucleic acid molecule of claim 1, wherein said nucleic acid molecule encodes a CMRP involved in the production of a fine chemical.

4. The isolated nucleic acid molecule of claim 1, wherein said nucleic acid molecule encodes a CMRP involved in the production of carbohydrates.

5. The isolated nucleic acid molecule of claim 1, wherein said nucleic acid molecule encodes an CMRP involved in the production of starch, cell wall polysaccharides and/or soluble sugars.

6. The isolated nucleic acid molecule of claim 1, wherein said nucleic acid molecule encodes a CMRP polypeptide assisting in transmembrane transport.

7. An isolated nucleic acid molecule from mosses selected from the group consisting of those sequences set forth in Appendix A (SEQ ID NO:1 to SEQ ID NO:177, odd integers), or a portion thereof.

8. An isolated nucleic acid molecule which encodes a polypeptide sequence selected from the group consisting of those sequences set forth in Appendix B (SEQ ID NO:2 to SEQ ID NO:178, even integers).

9. An isolated nucleic acid molecule which encodes a naturally occurring allelic variant of a polypeptide selected from the group of amino acid sequences consisting of those sequences set forth in Appendix B (SEQ ID NO:2 to SEQ ID NO:178, even integers).

10. An isolated nucleic acid molecule comprising a nucleotide sequence which is at least 50% homologous to a nucleotide sequence selected from the group consisting of those sequences set forth in Appendix A (SEQ ID NO:1 to SEQ ID NO:177, odd integers), or a portion thereof.

11. An isolated nucleic acid molecule comprising a fragment of at least 15 nucleotides of a nucleic acid comprising a nucleotide sequence selected from the group consisting of those sequences set forth in Appendix A (SEQ ID NO:1 to SEQ ID NO:177, odd integers).

12. An isolated nucleic acid molecule which hybridizes to the nucleic acid molecule of any one of claims 1 or 7 to 11 under stringent conditions.

13. An isolated nucleic acid molecule comprising the nucleic acid molecule of any one of claims 1 or 7 to 11 or a portion thereof and a nucleotide sequence encoding a heterologous polypeptide.

14. A vector comprising the nucleic acid molecule of any one of claims 1 or 7 to 11, or a portion thereof, or the isolated nucleic acid molecule which hybridizes to a nucleic acid molecule of any one of claims 1 or 7 to 11 under stringent conditions, or a portion thereof, and, optionally, a nucleotide sequence encoding a heterologous polypeptide.

15. The vector of claim 14, which is an expression vector.

16. A host cell transformed with the expression vector of claim 15.

17. The host cell of claim 16, wherein said cell is a microorganism.

18. The host cell of claim 16, wherein said cell belongs to the genus mosses or algae.

19. The host cell of claim 16, wherein said cell is a plant cell.

20. The host cell of claim 16, wherein the expression of said nucleic acid molecule results in the modulation of the production of a fine chemical from said cell.

21. The host cell of claim 16, wherein the expression of said nucleic acid molecule results in the modulation of the production of carbohydrates from said cell.

22. The host cell of claim 16, wherein the expression of said nucleic acid molecule results in the modulation of the production of starch, cell wall polysaccharides and/or soluble sugars from said cell.

23. Descendants, seeds or reproducable cell material derived from a host cell of claim 16.

24. A method of producing a polypeptide comprising culturing the host cell of claim 16 in an appropriate culture medium to, thereby, produce the polypeptide.

25. An isolated CMRP polypeptide from mosses or algae or a portion thereof.

26. An isolated CMRP polypeptide from microorganisms or fungi or a portion thereof.

27. An isolated CMRP polypeptide from plants or a portion thereof.

28. The polypeptide of any one of claims 25 to 27, wherein said polypeptide is involved in the production of a fine chemical.

29. The polypeptide of any one of claims 25 to 27, wherein said polypeptide is involved in assisting in transmembrane transport.

30. An isolated polypeptide comprising an amino acid sequence selected from the group consisting of those sequences set forth in Appendix B (SEQ ID NO:2 to SEQ ID NO:178, even integers).

31. An isolated polypeptide comprising a naturally occurring allelic variant of a polypeptide comprising an amino acid sequence selected from the group consisting of those sequences set forth in Appendix B (SEQ ID NO:2 to SEQ ID NO:178, even integers), or a portion thereof.

32. The isolated polypeptide of any of claims 25 to 27 or 30 to 31, further comprising heterologous amino acid sequences.

33. An isolated polypeptide which is encoded by a nucleic acid molecule comprising a nucleotide sequence which is at least 50% homologous to a nucleic acid selected from the group consisting of those sequences set forth in Appendix A (SEQ ID NO:1 to SEQ ID NO:177, odd integers).

34. An isolated polypeptide comprising an amino acid sequence which is at least 50% homologous to an amino acid sequence selected from the group consisting of those sequences set forth in Appendix B (SEQ ID NO:2 to SEQ ID NO:178, even integers).

35. An antibody specifically binding to a CMRP polypeptide of any one of claims 25 to 27, 30 to 31 or 33 to 34 or a portion thereof.

36. A test kit comprising a nucleic acid molecule of any one of claims 1 or 7 to 11, a portion and/or a complement thereof used as probe or primer for identifying and/or cloning further nucleic acid molecules involved in the production of carbohydrates or assisting in transmembrane transport in other cell types or organisms.

37. A test kit comprising a CMRP polypeptide-antibody of claim 35 for identifying and/or purifying further CMRP polypeptide molecules or fragments thereof in other cell types or organisms.

38. A method for producing a fine chemical, comprising culturing a cell containing a vector of claim 14 such that the fine chemical is produced.

39. The method of claim 38, wherein said method further comprises the step of recovering the fine chemical from said culture.

40. The method of claim 38, wherein said method further comprises the step of transforming said cell with the vector of claim 14 to result in a cell containing said vector.

41. The method of claim 38, wherein said cell is a microorganism.

42. The method of claim 38, wherein said cell belongs to the genus Corynebacterium or Brevibacterium.

43. The method of claim 38, wherein said cell belongs to the genus mosses or algae.

44. The method of claim 38, wherein said cell is a plant cell.

45. The method of claim 38, wherein expression of the nucleic acid molecule from said vector results in modulation of the production of said fine chemical.

46. The method of claim 38, wherein said fine chemical is selected from the group consisting of carbohydrates, cofactors and/or enzymes.

47. The method of claim 46, wherein said fine chemical is selected from the group consisting of starch, cell wall polysaccharides and/or soluble sugars.

48. A method for producing a fine chemical, comprising culturing a cell whose genomic DNA has been altered by the inclusion of a nucleic acid molecule of any one of claims 1 or 7 to 11.

49. The method of claim 48, comprising culturing a cell whose membrane has been altered by the inclusion of a polypeptide of any one of claims 25 to 27, 30 to 31 or 33 to 34.

50. A fine chemical produced by the method of claim 38 or 48.

51. Use of a fine chemical of claim 50 or a polypeptide of any one of claims 25 to 27, 30, 31, 33 or 34 for the production of another fine chemical.


SEQUENCE LISTING (181 sequences)

SEQ ID NO: 1 - DNA, 550 bp, Physcomitrella patens
  CDS (1)..(549): 18_ck32_c09fwd
agcaagcccaggattgcggcctggaacagtgatgagttgccgatttatgagccgggtttggacgatgttgtgaagtcgtgcaggggtaagaacttgttcttctcgacggaggttgagaagcacgtcgctgaggcggacattgtgttcgtgtctgtgaacactccaaccaagacccggggtttgggagctggtaaggctgcagatctgacttattgggaaagtgctgcgcgtatgattgctgatgtgtcgaagagcgataagatcgtggttgagaagtctaccgtgccggtgaaaaccgcagaggcgatcgagaagatcctgacgcataacaacaaggggattaactttcagatcttgtcgaacccggagtttttggccgagggtaccgccattgaagacttggacaagcccgatcgtgtgctgatcggtggacgcatgactccggagggacagaaagctgtggctgctttgaagctgtgtacgcacactgggtgccagaggaccgcattattactaccaacttgtggtctgctgactctccaagctcgcc

SEQ ID NO: 2 - protein, 183 aa, Physcomitrella patens
SKPRIAAWNSDELPIYEPGLDDVVKSCRGKNLFFSTEVEKHVAEADIVFVSVNTPTKTRGLGAGKAADLTYWESAARMIADVSKSDKIVVEKSTVPVKTAEAIEKILTHNNKGINFQILSNPEFLAEGTAIEDLDKPDRVLIGGRMTPEGQKAVAALKLCTHTGCQRTALLLPTCGLLTLQAR

SEQ ID NO: 3 - DNA, 547 bp, Physcomitrella patens
  CDS (1)..(546): 21_ppprot1_047_d02
ggtctggaaaatccaccaggagcacattgttgtttgcctcacgtagaaaatctgctggacgctcgtagtcccatggccgcccaagtatttttcaatggcagtttgctgggaggcattagttgcacttcaacacccagttcactttctgtgcctcgatcttcgctgacactacctgtgccaacttccttgcgcaagagtctttactctatggtaagctggaaggatttgctttctaggcaccgttcttttaagaggggtatgaggtcaagcgctttagctgattcagacttttcagtgaagaaagatgagatgcaaacgcaaagtatgtttccagctgcaagacgtggcaaggagaatgcggtttctacgaccactgtgaacgctacaagtgtacttccggaacgcaaaatcattcatgagacagcggtagttcatccggacgccttcataggtgagggggttgtcatcagcgcattttgtacagtgggacctggtgtttcaataggaaatggctgcaagttacatcctagtagtcacgtctgtggga

SEQ ID NO: 4 - protein, 182 aa, Physcomitrella patens
GLENPPGAHCCLPHVENLLDARSPMAAQVFFNGSLLGGISCTSTPSSLSVPRSSLTLPVPTSLRKSLYSMVSWKDLLSRHRSFKRGMRSSALADSDFSVKKDEMQTQSMFPAARRGKENAVSTTTVNATSVLPERKIIHETAVVHPDAFIGEGVVISAFCTVGPGVSIGNGCKLHPSSHVCG

SEQ ID NO: 5 - DNA, 536 bp, Physcomitrella patens
  CDS (2)..(163): 91_ppprot1_055_h04
cattctgcgaggcagtgcgcagaaagcgaaggaggtgctgggatggcagcctaaggtgcagttcaagcagctggtggcgatgatggtggatggtgatttggagaaggcgaagcgagagaaggtgcttgtggatgctggcttcattgactcgcaccagcagccctgaattttgggcaccgaatgaatagtgttaataattatatgaaacgaatggatataatatgacaggccttgcaatatatggttaatatattgatacatagtgatatgtcaacccgagagtacttcttcaattaggttatagccttagctttgccatgtaaggcttacaatatattcttcgctgccgcagtgcttagcacacaccaagtactagttccgagcaattttagtgggttgtttattcagcagaatgcactgacaccactcatctagaatataagcccgcattcgggtgcaaatcaatgctattctctgatgaggacgattttgccaacctgtgcaccctccttcgaaatgaatattcaatcttaaaa

SEQ ID NO: 6 - protein, 54 aa, Physcomitrella patens
ILRGSAQKAKEVLGWQPKVQFKQLVAMMVDGDLEKAKREKVLVDAGFIDSHQQP

SEQ ID NO: 7 - DNA, 658 bp, Physcomitrella patens
  CDS (3)..(284): 51_ppprot1_056_a05
tcgcgacggaggattcccacactgtggaggagtttctggaagaggcattcagctatgttggtctgaactggaaggaccatgtcgagattgatcccagatatttccgtccttcggaggtggacaatctgcgaggcagtgcgcagaaagcgaaggaggtgctgggatggcagcctaaggtgcagttcaagcagctggtggcgatgatggtggatggtgatttggagaaggcgaagcgagagaaggtgcttgtggatgctggcttcattgactcgcaccagcagccctgaattttgggcaccgaatgaatagtgttaataattatatgaaacgaatggatataatatgacaggccttgcaatatatggttaatatattgatacatagtgatatgtcaacccgagagtacttcttcaattaggttatagccttagctttgccatgtaaggcttacaatatattcttcgctgccgcagtgcttagcacacaccaagtactagttccgagcaattttagtgggttgtttattcagcagaatgcactgacaccactcatctagaatataagcccgcattcgggtgcaaatcaatgctattctctgatgaggacgattttgccaacctgtgcaccctccttcgaaatgaatattcaattcttaaaa

SEQ ID NO: 8 - protein, 94 aa, Physcomitrella patens
ATEDSHTVEEFLEEAFSYVGLNWKDHVEIDPRYFRPSEVDNLRGSAQKAKEVLGWQPKVQFKQLVAMMVDGDLEKAKREKVLVDAGFIDSHQQP

SEQ ID NO: 9 - DNA, 437 bp, Physcomitrella patens
  CDS (1)..(141): 05_ppprot1_090_a03
aggcccagagagcgtttggtttgccaaccaaaagtcaacttcaagcagttggttgctatgatggtagacggtgatttggagagagctaaacgtgagaaggttctagtggacaacggttatattgattctcaccaacagccttgaagcacatcaatccactcttcattcctatttttataccatgagaagatgaaaaaatgtgttctttaagtcccatgcattcattggctcaaatgccgaagatggtttaacagacgaccggatatccaagcaatcaataggagagatagatcgttaaatatctagtatcggtgccctggaaattagttgggatagtggaataagctttctgcaaggccaacagtgatcactctttcagaccatacatttatggtgaataccttgataatgctaacatttaccacgaaaaaaaaaaa

SEQ ID NO: 10 - protein, 47 aa, Physcomitrella patens
RPRERLVCQPKVNFKQLVAMMVDGDLERAKREKVLVDNGYIDSHQQP

SEQ ID NO: 11 - DNA, 522 bp, Physcomitrella patens
  CDS (3)..(194): 15_ppprotl_080_c02
atttcactcaagaacaataccaccacgagatcaggagatgtgatatacacagccagttactgaatgttatccgacaacagagccaacagaggttcgtggtaaatgttagcattatcaaggtattcaccataaatgtatggtctgaaagagtgatcactgttggccttgcagaaagcttattccactatcccaactaatttccagggcaccgatactagatatttaacgatctatctctcctattgattgcttggatatccggtcgtctgttaaaccatcttcggcatttgagccaatgaatgcatgggacttaaagaacacattttttcatcttctcatggtataaaaataggaatgaagagtggattgatgtgcttcaaggctgttggtgagaatcaatataaccgttgtccactagaaccttctcacgtttagctctctccaaatcaccgtctaccatcatagcaaccaactgcttgaagttgacttttggttgcaaccaaacgctctctggccttctgt

SEQ ID NO: 12 - protein, 64 aa, Physcomitrella patens
FTQEQYHHEIRRCDIHSQLLNVIRQQSQQRFVVNVSIIKVFTINVWSERVITVGLAESLFHYPN

SEQ ID NO: 13 - DNA, 614 bp, Physcomitrella patens
  CDS (1)..(318): 80_ppprot1_092_f10
atgttgcagcaggagaagccagatgattatgtccttgctaccgagagttcttatactgtcgaagagtttcttgaggaagccttcggttacgttggtcttaactggagagatcatgttgaaatcgatcccaggtatttccgtccttccgaagtggataatttgagaggttcagcacagaaggccagagagcgtttgggttggcaaccaaaagtcaacttcaagcagttggttgctatgatggtagacggtgatttggagagagctaaacgtgagaaggttctagtggacaacggttatattgattctcaccaacagccttgaagcacatcaatccactcttcattcctatttttataccatgagaagatgaaaaaatgtgttctttaagtcccatgcattcattggctcaaatgccgaagatggtttaacagacgaccggatatccaagcaatcaataggagagatagatcgttaaatatctagtatcggtgccctggaaattagttgggatagtggaataagctttctgcaaggcgaacagtgatcactctttcagaccatacatttatggtgaataccttgataatgctaacatttaccacgaaaaaaaaaaa

SEQ ID NO: 14 - protein, 106 aa, Physcomitrella patens
MLQQEKPDDYVLATESSYTVEEFLEEAFGYVGLNWRDHVEIDPRYFRPSEVDNLRGSAQKARERLGWQPKVNFKQLVAMMVDGDLERAKREKVLVDNGYIDSHQQP

SEQ ID NO: 15 - DNA, 701 bp, Physcomitrella patens
  CDS (2)..(487): 20_ppprot1_064_d07
ccaggcttacaggctgcagtataatttcgacgccatttctggaatgccgacaaacctctacggtccccacgacaatttccatcccgagaactcccacgtcttgccagccttgatcagacgctttcacgaggctaaggtgaacggcgctaaggaagtggttgtgtggggatcaggttccccattccgtgagtttcttcacgtggacgacttggcagaggcaacagtatttctgctgcagaattactccgcgcatgagcatgtcaacatgggcagtggctctgaggtctcaatcaaggaactcgccgaaatggtgaaggaagtggttggatttcaggggcagctgacatgggatacttctaagcctgatggaactccacgaaagctcatcgatagcagcaaacttgccaacatggggtggcaagcgagaattcccctcaaggaaggattggcagagacttacaaatggtactgtgagaactacaatgtctaggctattttattcggatcaaccttgaagcacctgtttttgaattcttactacgatagataaattcaagcggtggctatgtgaagcagtggtagctttgcaggatactgacctcgaggatatttatcacaattcattgcctgtttagtgggtactgcaaccttgtattgtgaggctgtcatggcaattttctttctagcatgctgactttaaa

SEQ ID NO: 16 - protein, 162 aa, Physcomitrella patens
QAYRLQYNFDAISGMPTNLYGPHDNFHPENSHVLPALIRRFHEAKVNGAKEVVVWGSGSPFREFLHVDDLAEATVFLLQNYSAHEHVNMGSGSEVSIKELAEMVKEVVGFQGQLTWDTSKPDGTPRKLIDSSKLANMGWQARIPLKEGLAETYKWYCENYNV

SEQ ID NO: 17 - DNA, 670 bp, Physcomitrella patens
  CDS (2)..(340): 41_ppprot1_069_g03
agggacagggattgtacgtgtcatactgggacggaagcagtgggctactcaaggtggtcgcatcaaaatcgactacgcgatgaacgcgccgtttattgctaatttgaacgggttccatcacatgtcggcttgccaggttccgagtgagagggacctcggaagttgcaaataccctgctgtggctccatgctgggacaggccatctgatcattaccttaccgcgaagcagaagaccgatttcgagtgggcgaacaacaacttctgtatctacgactactgcaaggacgaccagcgatttgcctcgacgggaaaaccagcagaatgcaacatcccactctattagatctcatactcttcttcgagcatctgtctgggccccagtggcattcattctaaataagtagcatcccacttagacatcgctaatttaattcattggtgttgctgtccccctgcagtggtccagcattgcaagatggggggttattgatcgagtcacgatgctcggggaactcgttgggaaagggtgctggagtggtcgcactgccgcctcccattgcattagatgtctagcggcgttcagcgaactgaaacatatttgtactagagtgcttaacaattcctttctcccagtttttaatactcgggatgacttattcttcaaaaaaaa

SEQ ID NO: 18 - protein, 113 aa, Physcomitrella patens
GTGIVRVILGRKQWATQGGRIKIDYAMNAPFIANLNGFHHMSACQVPSERDLGSCKYPAVAPCWDRPSDHYLTAKQKTDFEWANNNFCIYDYCKDDQRFASTGKPAECNIPLY

SEQ ID NO: 19 - DNA, 535 bp, Physcomitrella patens
  CDS (104)..(502): 48_ck10_h09fwd
ctggaactgtgttcgcattctacacctcttcggatgggaaaatggatgaccatgacgaaattgatattgaattcttggtaacgaaacctccaagcacatcactatgcagaccaatatcttcatcaacggtgtcggtgatagggaaatgcgccacaaccttgactggtttgacccatgcaccagccatcatgactacttcatcaagtggaattccaacattgtcgtagttggagttgatgatatccctctccgcgttcacatgaacaatgagaagaacggtctcccatattttaacaagggacagggattgtacgtgtcatactgggacggaagcagctgggctactcaaggtggtcgcatcaaaatcgactacgcgatgaacgcgccgtttattgctaatttgaacgggttccatcacatgtcggcttgccaggttccgagtgagagggacctcggaagttgcaaataccctgctgtgggctccatgctgggacagggcatctgatcattaccttaccgcgaagcagaagaccga

SEQ ID NO: 20 - protein, 133 aa, Physcomitrella patens
MQTNIFINGVGDREMRHNLDWFDPCTSHHDYFIKWNSNIVVVGVDDIPLRVHMNNEKNGLPYFNKGQGLYVSYWDGSSWATQGGRIKIDYAMNAPFIANLNGFHHMSACQVPSERDLGSCKYPAVGSMLGQGI

SEQ ID NO: 21 - DNA, 575 bp, Physcomitrella patens
  CDS (2)..(394): 18_ppprot1_055_c09
tgttcctggtggacgggccgtcgtgcgggtgttcaagaacctggaaggacaagtgccggggttcaagtacctgaaggatcaggcgatgatggtgtacgtgagcatctgggacggcagccagtgggctactcagggagggagggtgaagatcaactatgactcggctcccttcgtggcgcactacgactacttcgggctgaatgggtgcacggtggacccgaatgatggagcgaatggagtggcagcgtgccagtacagtccgtatgcgacgggacagaacaatgggaacagcaacccacagccctcgacaacgacgacgatgctgtacgactactgctatgacacgaacaggaacccgacgccgccaccagaatgcgcgtacaacaaggtagagtagagtagaggagtgcagatcagagcagcccatgtggggattggggattcttcgatgagtgggcgtaggggggcagcatttgcagagtagggcggaggagttgtaagtgtgtggaattggggagtgtattgatacattgttcttgaataaaagtggaaccgttggggctgcaaaaaaaaaa

SEQ ID NO: 22 - protein, 131 aa, Physcomitrella patens
VPGGRAVVRVFKNLEGQVPGFKYLKDQAMMVYVSIWDGSQWATQGGRVKINYDSAPFVAHYDYFGLNGCTVDPNDGANGVAACQYSPYATGQNNGNSNPQPSTTTTMLYDYCYDTNRNPTPPPECAYNKVE

SEQ ID NO: 23 - DNA, 612 bp, Physcomitrella patens
  CDS (3)..(431): 90_ppprot1_056_g12
acttgtacaccttccggtggaccaaggactcggtgctgttcctggtggacggggccgtcgtgcgggtgttcaagaacctggaaggacaagtgccggggttcaagtacctgaaggatcaggcgatgatggtgtacgtgagcatctgggacggcagccagtgggctactcagggagggagggtgaagatcaactatgactcggctcccttcgtggcgcactacgactacttcgggctgaatgggtgcacggtggacccgaatgatgcagcgaatggagtggcagcgtgccagtacagtccgtatgcgacgggacagaacaatgggaacagcaacccacagccctcgacaacgacgacgatgctgtacgactactgctatgacacgaacaggaacccgacgccgccaccagaatgcgcgtacaacaaggtagagtagagtagaggagtgcagatcagagcagcccatgtggggattggggattcttcgatgagtgggcgtaggggggcagcatttgcagagtagggcggaggagttgtaagtgtgtggaattggggagtgtattgatacattgttcttgaataaaagtggaaccgttggggctgcaaaaaaaaaa

SEQ ID NO: 24 - protein, 143 aa, Physcomitrella patens
LYTFRWTKDSVLFLVDGAVVRVFKNLEGQVPGFKYLKDQAMMVYVSIWDGSQWATQGGRVKINYDSAPFVAHYDYFGLNGCTVDPNDAANGVAACQYSPYATGQNNGNSNPQPSTTTTMLYDYCYDTNRNPTPPPECAYNKVE

SEQ ID NO: 25 - DNA, 621 bp, Physcomitrella patens
  CDS (237)..(620): 37_ppprot1_051_g01
cagctttagattgcaagagcaggttccctcaggacttcgaatctggatcgcgctcacagaaagtccacatgttagttgcctctcgtagtcgcgctgcttagtttcgacaggtttcaggctcctggaagtcttttgcaaccaggtttccgggccagcttgaacagcacttgttcggtactgtttagaagttgaactttgaagtgcgcaacgagatagtatttcgagaagtatcgacaatgggttccttgggaagacaaggatgtttattggtcggtgtcttgttttacttgagcatggctatcggcgctcaagctcagagttacccaggacttcaggctgcattcaattcttggacgccgaagcagattatcccggataagaatggaaggaaagtgcaactcgtgcttaacaattcatcttcggcatatactggcatgggatctaagcaatcgtggctgtttgggggtatcggggcctggatcaagctccccgctaacgattccgctggaactgtcaccacattctacatgtcatctactgggccgaagcattgcgagttcgacttcgagttcctaggcaactccagcggccaaccttaccttctccataccaacatcttcg

SEQ ID NO: 26 - protein, 128 aa, Physcomitrella patens
MGSLGRQGCLLVGVLFYLSMAIGAQAQSYPGLQAAFNSWTPKQIIPDKNGRKVQLVLNNSSSAYTGMGSKQSWLFGGIGAWIKLPANDSAGTVTTFYMSSTGPKHCEFDFEFLGNSSGQPYLLHTNIF

SEQ ID NO: 27 - DNA, 570 bp, Physcomitrella patens
  CDS (102)..(569): 35_mm14_f03rev
atttgatcgcaacgatttcacctagagcggtggagtgattttcagctgctgcctgataggaaggattgtctaacgggattgggggaacgtgcaatctagcaatggggtcgctcgggggttcgcgtagcaccctgctgattttgctgctactgtgtttgagcttggctgttggcggtcgcgcccaaacgcttgctcagcagttcactccgtggactgaaaatgcgaggttcactactgacactcaaatgcagctcaccttggatcaacgctatgcagctggggcaggatccgtgaacgtttggacgtacgtcgacatcagcgcgtacataaagatgccgccattcgattccgctggtactgtgacaacgttctacatgtcgtctcagggtgaccagcattacgagctggacatggagtttttgggaaacactagcggacagcccttcctgcttcacacgaatgtgttcgttgatggggttgggggtcgcgagcagcaaatgtacctgggattcgacccctctgctgacttccactactacagattccggtggagtaaggatatggttgttt

SEQ ID NO: 28 - protein, 156 aa, Physcomitrella patens
MGSLGGSRSTLLILLLLCLSLAVGGRAQTLAQQFTPWTENARFTTDTQMQLTLDQRYAAGAGSVNVWTYVDISAYIKMPPFDSAGTVTTFYMSSQGDQHYELDMEFLGNTSGQPFLLHTNVFVDGVGGREQQMYLGFDPSADFHYYRFRWSKDMVV

SEQ ID NO: 29 - DNA, 597 bp, Physcomitrella patens
  CDS (1)..(432): 96_ppprot1_081_h12
tactacagattccggtggagtaaggatatggttgttttctacgtcgataacaaacccgtccgagttttcaagaatctggaaggcacggtaccggggactaaatacctgaaccagcaagcaatgggggtgtacataagcatctgggacggtagcagttgggccacgcaaggagggcgtgtgcccatcaactgggcttccgctccattcactgcgacgtaccaggacttcgcactgaatgggtgcgtggtagaccccaacgatcccaatggagttgcagcatgccagaactctccgtatgcaaccggagcagccttgagcaatcaggaagtttatgagttggggcagaacaaagcttacatgatgaaatacgactactgcgacgacagggttcgatacccagatgtgccacctgaatgtccttacaacaacgtgtagaatacggaatgagtcgtgtacatgttacgtgctagctatttggggcgtggttgcctagtgaagatatagttgcgtagaggtcatctgattcttttgtatattaattgtacgcggatgtcgattcttgaaaggaagatttggttgtagctcatattattaaaa

SEQ ID NO: 30 - protein, 144 aa, Physcomitrella patens
YYRFRWSKDMVVFYVDNKPVRVFKNLEGTVPGTKYLNQQAMGVYISIWDGSSWATQGGRVPINWASAPFTATYQDFALNGCVVDPNDPNGVAACQNSPYATGAALSNQEVYELGQNKAYMMKYDYCDDRVRYPDVPPECPYNNV

SEQ ID NO: 31 - DNA, 570 bp, Physcomitrella patens
  CDS (2)..(517): 96_ck7_h12fwd
gaagcggtcaatgaacaactccgggacgccgatgcggcctggcgtggagttcgacgcctacattgtgagcctgtacgacgagaatttgcgcccgacaccgcccgcatcggcgcagcattgggggctattctacgtgaacgggacgcacaagtacgggtttaactatttgaatgggagtgatgtgcctggcggcggtggtggcggaggcggcggtaatggttccactcctggatcacctcctggaagcggtggtggcggtggaggaggtagcagtggtggcgcaatcccgggccagaaagtgtggtgcattgcaaagtcaagcgcttctaataccagcttgatacagggaattgactgggcttgtggagcggggaaggccaagtgcgaccccattcaacgtggcggtgactgttacttgcccgacacaccctactcgcacgcctcctatgcgtttaacatccactaccactggttccaaacggatccccggtcgtgtatattcggcggagacgcagactgacttatgtcgatcccactatggaagctgctactacgtaccgagtggtgcca

SEQ ID NO: 32 - protein, 172 aa, Physcomitrella patens
KRSMNNSGTPMRPGVEFDAYIVSLYDENLRPTPPASAQHWGLFYVNGTHKYGFNYLNGSDVPGGGGGGGGGNGSTPGSPPGSGGGGGGGSSGGAIPGQKVWCIAKSSASNTSLIQGIDWACGAGKAKCDPIQRGGDCYLPDTPYSHASYAFNIHYHWFQTDPRSCIFGGDAD

SEQ ID NO: 33 - DNA, 553 bp, Physcomitrella patens
  CDS (3)..(515): 37_mm21_g0lrev
gcgccaccaccaagggcaccaccatcctgggcggcatcaggcaagtcataggtcgtaactctgaggttgtataccagcccaacccctctgccggatatgctaagggcaagggcttcgagtacgccattgtcgttgttggtgagcaaccctacgctgaggtgaacggtgacaacctcaacaatctcaacatgccggcgccataccccgccctaatcaaggatacctgctccaatgttgcgtgcgtggtagtcatgatctctggcagaccgcttgtggttgagccctacctgggctacatgaacgcgtttgtcgctgcatggcttccaggatctgaaggacgtggagttgccgaagtgctgttcggcaactacgaattttccgggaggctatcaaggacgtggttccggcgcgtcgatcagctgcctatgaacgttggcgatcgatactacaacccgttgttccccttcggatacgggatgaagatgggcctcaaaacgtgcctagttccttcgagttgaattatgtattccctgtagtgtgcaattgatcgatg

SEQ ID NO: 34 - protein, 171 aa, Physcomitrella patens
ATTKGTTILGGIRQVIGRNSEVVYQPNPSAGYAKGKGFEYAIVVVGEQPYAEVNGDNLNNLNMPAPYPALIKDTCSNVACVVVMISGRPLVVEPYLGYMNAFVAAWLPGSEGRGVAEVLFGNYEFSGRLSRTWFRRVDQLPMNVGDRYYNPLFPFGYGMKMGLKTCLVPSS

SEQ ID NO: 35 - DNA, 240 bp, Physcomitrella patens
  CDS (1)..(240): 10_ppprot1_085_b08
cggacagactaccccgtgattgaaaatatatcaattgagaatgtggtgggggaaaacataactcatgctggcctatttctgggacttcctgagtctcccttccacaacattcacctggccaacatagctcttgacgtcaagtctgaatccgacgactggaactgctcatcagtcgctggaacctacttcttcgtttggccccagccatgctcagacttcactaaggaggagcaaaagacc

SEQ ID NO: 36 - protein, 80 aa, Physcomitrella patens
RTDYPVIENISIENVVGENITHAGLFLGLPESPFHNIHLANIALDVKSESDDWNCSSVAGTYFFVWPQPCSDFTKEEQKT

SEQ ID NO: 37 - DNA, 496 bp, Physcomitrella patens
  CDS (2)..(496): 16_mm6
ggctgacgggacacactggcctggaacatggaatcagtctggcaaagagcatggtagaggagaccacgcaggcatcattcaggtgatgctcgcgcctcccactgccgagcctctgatgggcagcagcgacgaagagaacatcatcgacaccacggacgtcgatatccgtctgccgatgcttgtctacatgtctcgcgagaagcgccggggatacgatcacaacaaaaaggccggagctatgaatgcacttgtgcgaacgagtgccgtaatgtctaacgggcccttcattctcaatctggattgtgatcactacatcttcaattctctcgctatccgagaggccatgtgcttcttcatggacaagggcggtgaccgcattgcatacgtgcagttccctcagcgttttgagggcgtggatccgaacgatcgatacgccaaccacaacaccgtcttcttcgacgtgaatatgagggctctggatggactgcaagggcct

SEQ ID NO: 38 - protein, 165 aa, Physcomitrella patens
ADGTHWPGTWNQSGKEHGRGDHAGIIQVMLAPPTAEPLMGSSDEENIIDTTDVDIRLPMLVYMSREKRRGYDHNKKAGAMNALVRTSAVMSNGPFILNLDCDHYIFNSLAIREAMCFFMDKGGDRIAYVQFPQRFEGVDPNDRYANHNTVFFDVNMRALDGLQGP

SEQ ID NO: 39 - DNA, 480 bp, Physcomitrella patens
  CDS (3)..(479): 83_mm10_f06rev
gcatcacgctggaggaatggtggcgaaatgagcaattctgggtgatcggtggcacgagcgctcacttagctgccgtctttcagggtttcctgaaagtcatcgccggggtcgacatctccttcacgcttacatccaaggcaactggggacgagggggatgacgagtttgccgatctgtacgtggtgaagtggagcgctctcatgatccctcccatcaccatcatgatcaccaacgtagtggctattgcggtgggcacctcgcgccagatttacagcaccatcccggagtggagcaagctcatcggcggcgtcttcttctccttgtgggtgctctctcatctctacccctttgccaagggcctcatgggccgcaagggcaaaactccgaccattatctacgtgtggtcaggtttgctctccgtcatcatctccctcatgtgggtgtatataaatccgccttcaggaacttctgtcactgggg

SEQ ID NO: 40 - protein, 159 aa, Physcomitrella patens
ITLEEWWRNEQFWVIGGTSAHLAAVFQGFLKVIAGVDISFTLTSKATGDEGDDEFADLYVVKWSALMIPPITIMITNVVAIAVGTSRQIYSTIPEWSKLIGGVFFSLWVLSHLYPFAKGLMGRKGKTPTIIYVWSGLLSVIISLMWVYINPPSGTSVTG

SEQ ID NO: 41 - DNA, 410 bp, Physcomitrella patens
  CDS (1)..(345): 09_mm10_b02rev
atctacgcagatctttacgtagtgaagtggacatctctcatgattcctcccatcactatgggtctcaccaacatcatcgccattgctgtaggcgtctcccgaaccatctacagcgagatccccgagtggagcaagctcattggaggcgtgtttttctctctgtgggtgttgttccatctctaccccttcgccaagggtctcatgggcaaggggggcaagacgcccaccattatttacgtctgggccggtttgttatccgtcatcatatccctactctggctctacatcagtccctctgcgaacaggacagcacaagctggtgacggcggtggctttcagttcccctgaacactccaaattggtgttcatgttacgccagcacattcccgatgtgccgatcaattttttgc

SEQ ID NO: 42 - protein, 115 aa, Physcomitrella patens
IYADLYVVKWTSLMIPPITMGLTNIIAIAVGVSRTIYSEIPEWSKLIGGVFFSLWVLFHLYPFAKGLMGKGGKTPTIIYVWAGLLSVIISLLWLYISPSANRTAQAGDGGGFQFP

SEQ ID NO: 43 - DNA, 642 bp, Physcomitrella patens
  CDS (3)..(521): 67_mm22_d04rev
gacggggtgtctatccagatgggttgtttagaatgctgatggagtttcataaacgctatcaaaagcacaatatgaaatttattattacagagaacggtgtgtcagacgccaccgactatattcgtcgaccatatcttattgagcatctacttgctgtccgagcagcaatggaccagggtgtgcgcgttcaaggttactgcttctggacaatctcagacaactgggaatgggccgatgggtacggtccaaagttcggtctctgtgctgtggatcgacacaaggaccttgcgcgtcacccccgtccttcctatcatctttactctgaggtgtcaaaaactgggaaaataacgaagaaacagaggctggctgtatgggaagatcttcaggaccaagctaggcagagcaagatgagaccattttgtcgagaaaccaatgatcagggcctcatgtttgcagggggtctggatgtgccaatggatcgaccttcgctgttcgagattggcgctttggaaagtacgaggtagaaggtcttcaagatccgttaagctcttttgttcgttacttccgtggagcgaaccccttcaggaagaagacaaacagcagaagaagtcaatctcgaacccagcactcactgcagcttag

SEQ ID NO: 44 - protein, 173 aa, Physcomitrella patens
RGVYPDGLFRMLMEFHKRYQKHNMKFIITENGVSDATDYIRRPYLIEHLLAVRAAMDQGVRVQGYCFWTISDNWEWADGYGPKFGLCAVDRHKDLARHPRPSYHLYSEVSKTGKITKKQRLAVWEDLQDQARQSKMRPFCRETNDQGLMFAGGLDVPMDRPSLFEIGALESTR

SEQ ID NO: 45 - DNA, 564 bp, Physcomitrella patens
  CDS (1)..(306): 73_ck12_e04fwd
attgaatgggataagggtaaagctgtggagtttctgcttaagtctcttgggtttcaagatacakatgatctaattccactatatcttggagatgataagactgatgaagatgcattcaaggtagtcaattcgacaaaatacggctgcagtattttggtgtcttctgtagccaaaccgactgaagcaaaattttctctccgagatccatctgaggtcatggggtttttatgcaagcttgtgcactgggaaaagtgcagacaggatcctaacagcatttacatggatcgtaacttctcccagataccctaagatttttctaatatctgggtggttaagctttgttcctaacctcccgtgtgtataactacctatcgcagttacaataaccttcaaacactcccaccatgtgaaagaagactacctgtcaaaccacctcatttcggtcagaccaacccttattctgctcattatttctagtttgccctctgctaaaacagtcgattcagttgacatttgaactgaatcctcagtaaccttgatggtatgtaatcaagaaggaaatag

SEQ ID NO: 46 - protein, 102 aa, Physcomitrella patens
  VARIANT 22: Amino acid residue 22 may be Asp or Tyr.
IEWDKGKAVEFLLKSLGFQDTXDLIPLYLGDDKTDEDAFKVVNSTKYGCSILVSSVAKPTEAKFSLRDPSEVMGFLCKLVHWEKCRQDPNSIYMDRNFSQIP

SEQ ID NO: 47 - DNA, 520 bp, Physcomitrella patens
  CDS (1)..(519): 63_ck23_c05fwd
agagatagtgtgcaactcgtattcgattacttctgcgcaagaacgccacgatcttttgtagagaagcgagagacatctttggtttggaattataaatatgctgatttggagtttggaagggtccaagcacgaaacatgcttcaacatctatggacagggcccatctccaatgcggctgtggatgtcgttcaaggtcaaaagtcagtggaggtccgtcctattggtgtttcaaagggtgcagcaatcgatcgaattgtatttgaaattatacgcagcaagcatcccagcagttcacagacaccaaatattcagttcgactttgtcatgtgcctgggccattttttgagcaaggatgaggatgtgtatgcttatttcgatccggataacgcatttgatagagataatcggtgcagtaatggaaagctggaaaggaaccttgatcgacggttgtctgcaaagtcatgtgatcgtgcatcggtaaagaataataaatcgagcgtggttcgaacgaagacttacc

SEQ ID NO: 48 - protein, 173 aa, Physcomitrella patens
RDSVQLVFDYFCARTPRSFVEKRETSLVWNYKYADLEFGRVQARNMLQHLWTGPISNAAVDVVQGQKSVEVRPIGVSKGAAIDRIVFEIIRSKHPSSSQTPNIQFDFVMCLGHFLSKDEDVYAYFDPDNAFDRDNRCSNGKLERNLDRRLSAKSCDRASVKNNKSSVVRTKTY

SEQ ID NO: 49 - DNA, 541 bp, Physcomitrella patens
  CDS (3)..(539): 80_ck30_f10fwd
ggttcggattaggtttccggatcgtagctctggatccgagctttaagaagttgcgcaccgaacttattgtcggagcatacgggaagagtgccaccagagccttacttttggattatgatggtacagtgatgcctacatcacacgaagagagtcctagtcccgaggttttggatcttttaaatacgctttgcaacgacccaaagaacacacttttcatcgtaagtgggcgtccacggaataagcttggggaatggtttagttcctgcgagttacttggtcttgctgctgaacatggatacttttacaggtggcgacgggactcggattgggatacttgtagaccacaaagtgtttcagagtgggatcggttgagtgttgttgagcgtgaagcgccgtccacgagctttgattggaaactgattgcagggccagtcatgcaactttacaccgagtcaacagatggctcctatattgaggccaaagaaagtgcattggtgtggcattatcgagatgcagaccatgactttggagcttggcaggc

SEQ ID NO: 50 - protein, 179 aa, Physcomitrella patens
FGLGFRIVALDPSFKKLRTELIVGAYGKSATRALLLDYDGTVMPTSHEESPSPEVLDLLNTLCNDPKNTLFIVSGRPRNKLGEWFSSCELLGLAAEHGYFYRWRRDSDWDTCRPQSVSEWDRLSVVEREAPSTSFDWKLIAGPVMQLYTESTDGSYIEAKESALVWHYRDADHDFGAWQ

SEQ ID NO: 51 - DNA, 473 bp, Physcomitrella patens
  CDS (187)..(471): 46_ck2_h08fwd
cagccatcgaatgctcatcggctctgctcgattcctttagagcttaatcgcggatcggcggcggcagcagcagcagcaccgagtgcagcgagcccatccatctcgttgcaacgcaggaactggagcactccgagtcgtagcgatttcgagagcttcgttgcgcgcgagtgcttgtgttcgggagcaatggcattggctgcgtgcagggctgcgcactccgttgcgggggcttcgccgtcgtctctcgctgctgctgctgccaaaccctcttcgtcgctcgcgcgcccccaattcgctggactgcgccgtgctgatgtcgccaacgagtcatcgttcggggcagtcttgtctcaacggttgcagagtgcgggtacagggagcaggggagtcgtctccatggctggaactggaaagttcttcgtcgggggcaactggaagtgcaatggcacgactgagagcattaagaagcttgt

SEQ ID NO: 52 - protein, 95 aa, Physcomitrella patens
MALAACRAAHSVAGASPSSLAAAAAKPSSSLARPQFAGLRRADVANESSFGAVLSQRLQSAGTGSRGVVSMAGTGKFFVGGNWKCNGTTESIKKL

SEQ ID NO: 53 - DNA, 367 bp, Physcomitrella patens
  CDS (1)..(366): 83_bd06_f06rev
tgccgactcagatcagctatcgctgcttctttttctgctcccctcgcgtctgcccctgccttctccggcctccgtcgcctccctcttgctcccgcttcgtctcccgctttcggtgtcgtcttctctttgagtgaggggaaggggcacagaggtgtcgtcaccatgactggggccgggaagtttttcgttggcgggaactggaagtgcaatggcacaactgagtcgatcaagaagctcgtggaggatttgaacagtgcccaaattgaggacgacgttgatgtcgtcgtcgctcccccgtttttgtatatcagccaggtggtcgggtctttgacggaccgcattgaggtctccgctcagaactcttggg

SEQ ID NO: 54 - protein, 122 aa, Physcomitrella patens
CRLRSAIAASFSAPLASAPAFSGLRRLPLAPASSPAFGVVFSLSEGKGHRGVVTMTGAGKFFVGGNWKCNGTTESIKKLVEDLNSAQIEDDVDVVVAPPFLYISQVVGSLTDRIEVSAQNSW

SEQ ID NO: 55 - DNA, 333 bp, Physcomitrella patens
  CDS (1)..(333): 19_ck1_d01fwd
cttgcagctgtgggcaccaccgatttcggtgggttcacggtggaagtaattgaccctgtggaggattatctagaattgctgaaggaagtattcgactttgacttgatccgcagtctccttgccaggccgaacttcaggtttaagtttgacgccatgcacgctgtgactggcgcctacgccaagactatctttgtcgataccttgggcgcatcagaagactccattattaacggcattcccaaggacgatttcggagggggccaccccgatcccaacctgacgtacgcccacgagctcgttgatatcatgtatggccccgatgctcctggtttt

SEQ ID NO: 56 - protein, 111 aa, Physcomitrella patens
LAAVGTTDFGGFTVEVIDPVEDYLELLKEVFDFDLIRSLLARPNFRFKFDAMHAVTGAYAKTIFVDTLGASEDSIINGIPKDDFGGGHPDPNLTYAHELVDIMYGPDAPGF

SEQ ID NO: 57 - DNA, 212 bp, Physcomitrella patens
  CDS (1)..(210): ll_ck_19_b03
cttcatcctatgactgctaatcgctccaatcgagcgatcaggaaaggtgtcacttcacctaggttgcattgcaccacttgccaagccagagatatggacgacttggtggtatgctttggagagctcctgatagattttgtgcccactgtgggcggtctatcgcttgctgaagctcccgcattcaagaaagcgcctggaggtgcacctgccaa

SEQ ID NO: 58 - protein, 70 aa, Physcomitrella patens
LHPMTANRSNRAIRKGVTSPRLHCTTCQARDMDDLVVCFGELLIDFVPTVGGLSLAEAPAFKKAPGGAPA

SEQ ID NO: 59 - DNA, 677 bp, Physcomitrella patens
  CDS (2)..(394): 56_ppprot1_061_bl0
aaaacaccacattggaattcgaaagattgtagtggaggtttgcgatgtggtgtgtaagagaggtgctagactggcaggtgcgggaattgtaggaatattgaagaaaattggaagggatggaagtgcggcgaatggggttatcaagcgtaacctgtttgaacagagtgacatgaatggttaccatgacgacgaccctatgcaatatacatcagacgtgaaaaccgttgttgctatagacggtggtttgtatgaacactacaccaagttccgagaatacatgcaagatgctgtgtttgaacttcttggagaagcatcaaagaatgtctccatacagctttccaaagatggatcaggcattggagcagcccttcttgctgcatcgcatgccgagcatctttcttcttgataacgatgaaaccaactacagtcttgtgaaatatgagtctttgctgattgaaacctcttagttctaatgttagaaatgtgtataccaatccgtcaagggggtgcgagttagcttcctttggagcccttggtttatcggctcgccatttctgtagaaaggttcgctttttttaatcattaatcacgcatctcggcagctcatgtttccaagagtaacttgggacaacagatcccctggctgcatacgtaagcttatttgaaaaaaaaaaaaa

SEQ ID NO: 60 - protein, 131 aa, Physcomitrella patens
KHHIGIRKIVVEVCDVVCKRGARLAGAGIVGILKKIGRDGSAANGVIKRNLFEQSDMNGYHDDDPMQYTSDVKTVVAIDGGLYEHYTKFREYMQDAVFELLGEASKNVSIQLSKDGSGIGAALLAASHAEH

SEQ ID NO: 61 - DNA, 630 bp, Physcomitrella patens
  CDS (1)..(348): 18_ppprotl_064_c09
gacgttccgagatctcgtttccttcccgtcaaagctacctcagatttgctgttggttcagtcggacctctacacagttcaagatggtgcagtggtacggaaccctgccagaaccaaccccgaaaacccctccattgatctcagtagcgaattcaaaaaggtgggtgatttcttgaagcgtttcaaatcaattcccagcatccttgaattggagagcttgaaggttagcggaaacgtatggttcggcaaagacattgtcctcaagggtaaagtggttgttgaagcagctaagggagagaaagtggaagtgcctgacgaagctgttcttgataacacggtcgttaagaactagattattttatgaagtaactgttgctgtaacgtacaccgacttgtaagccttccatctgatttaggagatgagctactccgcaccgcttcagttagtgtagccaagttcattgtcatggtggtatcccatccatcagagattatctctgtttcaatgtgttctgggcatatcatctggtcaagttgcgatagtgatatgttagatcgacatgacgaattattttgctggatctgagactttcttgttgcgctggtttctattccagaaataataactttg

SEQ ID NO: 62 - protein, 116 aa, Physcomitrella patens
DVPRSRFLPVKATSDLLLVQSDLYTVQDGAVVRNPARTNPENPSIDLSSEFKKVGDFLKRFKSIPSILELESLKVSGNVWFGKDIVLKGKVVVEAAKGEKVEVPDEAVLDNTVVKN

SEQ ID NO: 63 - DNA, 496 bp, Physcomitrella patens
  CDS (2)..(496): 76_ck27_e11fwd
ctttccacgcatgacgaggccgcagtctattgggaatggtgtacaatttctgaatcggcacctatcttcgaggttgtttcgagacgcagacagcatggaaccacttgtcgagtttatgcgtgttcataaatataacgatcagactttgttgctgaatgagagtattactaacgtcgtcaggcttcgaccagctcttataaaagccgaagaatatttgatcaagcttccgaacgaccaaccgttaaaggatttctactccaagttgcaagaactggggctggagagaggctggggtgacacagctggacgcgtgttggaaatgatccatttgctgctagaccttttgcaagctcctgatcccgacatcttagagaagtttttggctcgcataccgatagtattcagtgtggccattatttcgcctcacggatacttcgggcagtctaacgttctaggaatgcccgatacgggaggacaagttgtatatatattggac

SEQ ID NO: 64 - protein, 165 aa, Physcomitrella patens
FPRMTRPQSIGNGVQFLNRHLSSRLFRDADSMEPLVEFMRVHKYNDQTLLLNESITNVVRLRPALIKAEEYLIKLPNDQPLKDFYSKLQELGLERGWGDTAGRVLEMIHLLLDLLQAPDPDILEKFLARIPIVFSVAIISPHGYFGQSNVLGMPDTGGQVVYILD

SEQ ID NO: 65 - DNA, 548 bp, Physcomitrella patens
  CDS (1)..(375): 71_ck18_d06fwd
ggtgatctccaggagcggacgtccatcttcttccatttgatacacgatggcaagcaccagaactggaagacgctcttctgcggcgaccagagccaatcctccttgcagcaggacgtcgacaagacggtgtatgggtcctacgtgcgcgtggatgacagcgacaaggtgctgtccgtgcgcattctcgtcgaccactccatcgtggagagcttcgcccaaggcggccgcacggtaatgacatccagagtatacccggagctggcggtgaaagacgccgctcacgtgtttttgttcaacaacggtactgagcccgtgacagtgaaatcggtatccacctgggagatgaagagtgtcaacatcaagttttacaaaccttgatttgcacgctctttccgcttaactccggttgataactagccgaaatgttgctttcccaatttagaattgtttggacatcctcgaagctcaagctgctgcatgcatacagagatattcaaaatcatgtacacatctccacttgtaataaaacataataaaccactctgttt

SEQ ID NO: 66 - protein, 125 aa, Physcomitrella patens
GDLQERTSIFFHLIHDGKHQNWKTLFCGDQSQSSLQQDVDKTVYGSYVRVDDSDKVLSVRILVDHSIVESFAQGGRTVMTSRVYPELAVKDAAHVFLFNNGTEPVTVKSVSTWEMKSVNIKFYKP

SEQ ID NO: 67 - DNA, 496 bp, Physcomitrella patens
  CDS (1)..(297): 94_ck14_h11fwd
tgcggcgaccagagccaatcctccttgcagcaggacgtcgacaagacggtgtatgggtcctacgtgcgcgtggatgacagcgacaaggtgctgtccgtgcgcattctcgtcgaccactccatcgtggagagcttcgcccaaggcggccgcacggtaatgacatccagagtatacccggagctggcggtgaaagacgccgctcacgtgtttttgttcaacaacggtactgagcccgtgacagtgaaatcggtatccacctgggagatgaagagtgtcaacatcaagttttacaaaccttgatttgcacgctctttccgcttaactccggttgataactagccgaaatgttgctttcccaatttagaattgtttggacatcctcgaagctcaagctgctgcatgcatacagagatattcaaaatcatgtacacatctccacttgtaataaaacataataaaccactctgttttggcaaaaaaaaaaaaaaaaaaaaaa

SEQ ID NO: 68 - protein, 99 aa, Physcomitrella patens
CGDQSQSSLQQDVDKTVYGSYVRVDDSDKVLSVRILVDHSIVESFAQGGRTVMTSRVYPELAVKDAAHVFLFNNGTEPVTVKSVSTWEMKSVNIKFYKP

SEQ ID NO: 69 - DNA, 390 bp, Physcomitrella patens
  CDS (3)..(281): 25_bd07_e01rev
gtaacgtgattgtgtttagacctgatggaggttctggaggttgctcgggtcattggtacgggtacgtcactcctgatgatgtcccagagataatggagaagcacattggacttggcgaggtggtgggtcggctttggaggggtcagatgggattgactgaggatgagcagaaggaagttcagcagaaaaggaacccctccagtaatctaactcaggagggcactaaaccagaagggaaggtggatgcagcttcaacatctactggaggcaactgctcctattaggaaaaagcctcctcctcctctacttccacgcattgggtcacgcgacctgcaccaaatgtgtggggacgaacaggaagaagaaggtccacttaaaccgtacgaggag

SEQ ID NO: 70 - protein, 93 aa, Physcomitrella patens
NVIVFRPDGGSGGCSGHWYGYVTPDDVPEIMEKHIGLGEVVGRLWRGQMGLTEDEQKEVQQKRNPSSNLTQEGTKPEGKVDAASTSTGGNCSY

SEQ ID NO: 71 - DNA, 415 bp, Physcomitrella patens
  CDS (1)..(414): 66_ppprotl_075_cl2
ctgacgaagtcggagactgttgccatgctgaactctgcggggctgtctcacatggagttcgacgctcttatttgcagcagtggaagtgaggtgtactatcctgcctcaattcaagacgatagcgtcacaacggacaacagcgacttgcatgcagacgaggactacaaaagccacattgactatcgatggggctacgaaggcctccgcaagaccatggcgcgcttgaacacacctgacactgagagcggtagcaacgacaagatctggaccgaggatacagcgaactgcaactctcactgcctggcctacacggtgaccaactcggacatcgcccctactgtggatcagctgcgacagcgtctgcggatgcggggactacgatgccacgtgatgttttgcaggaatgcttcacggt

SEQ ID NO: 72 - protein, 138 aa, Physcomitrella patens
LTKSETVAMLNSAGLSHMEFDALICSSGSEVYYPASIQDDSVTTDNSDLHADEDYKSHIDYRWGYEGLRKTMARLNTPDTESGSNDKIWTEDTANCNSHCLAYTVTNSDIAPTVDQLRQRLRMRGLRCHVMFCRNASR

SEQ ID NO: 73 - DNA, 576 bp, Physcomitrella patens
  CDS (1)..(576): 50_mm15_al0rev
ggcaaaggcgtcgcaatcgacggcacgcgtttacctttcgaagcgggggagattgaatttggggagcccggcacgaacggccagcacagcttctaccagctgatccaccaggggcgcaccatcccctgcgactttattggctccgtgaagagtcagaagcctatctacatgatcggggagaaggtgagcaaccacgacgagctcatgtcaaatttcttcgctcaagcagatgctctcgcctacggcaagacgcgggaggatctgcaagccgagaatgtgaaggagtctctagtccctcacaaggttttcactggcaatcgcccctccttgagcattctcctccccgcgctcaacgcctacaccgttggccagctgctatccctctacgagaaccgaatcgcagtacagggattcgtgtggggcattaactcattcgaccaatggggcgtggagctcggcaaaagcttggctaccaaggtgcgctcgcagctgaacgaagcacgcacgaaggacgctcccgtgcaaggattcaactacagcaccacgtatctctttgaagcactacttaaaggggaa

SEQ ID NO: 74 - protein, 192 aa, Physcomitrella patens
GKGVAIDGTRLPFEAGEIEFGEPGTNGQHSFYQLIHQGRTIPCDFIGSVKSQKPIYMIGEKVSNHDELMSNFFAQADALAYGKTREDLQAENVKESLVPHKVFTGNRPSLSILLPALNAYTVGQLLSLYENRIAVQGFVWGINSFDQWGVELGKSLATKVRSQLNEARTKDAPVQGFNYSTTYLFEALLKGE

SEQ ID NO: 75 - DNA, 476 bp, Physcomitrella patens
  CDS (3)..(476): 70_mm10_d11rev
ccacttttcgccgaatgtacaatcagtggccctttttccgtgtgactatcgacttggtggagatggtgtttgccaagggtgatccacgtattgccgctttatatgatgacttgcttgtgtcggacgagctgaaaccgcttggcgaggagctgaggcagaagtacaacgagacaagagatctccttctgaagataacattccacgatgaaatcttgcaagggaacccgtcgctgaagcaacggctgcggcttcgagaaccctacatcacggctctcaacgtgcagcaggcgctagtgctgaagaagatgcgtgatcagggcttgcagttctgtgcattacaaaacagcagcaaggaccaatccgacataccaacaacacccaagcgtgctgcagagctggttgaactgaaccccacaactgagttcccacccggactggaggatactctcattctcaccatgaagggtatcgcggcc

SEQ ID NO: 76 - protein, 158 aa, Physcomitrella patens
TFRRMYNQWPFFRVTIDLVEMVFAKGDPRIAALYDDLLVSDELKPLGEELRQKYNETRDLLLKITFHDEILQGNPSLKQRLRLREPYITALNVQQALVLKKMRDQGLQFCALQNSSKDQSDIPTTPKRAAELVELNPTTEFPPGLEDTLILTMKGIAA

SEQ ID NO: 77 - DNA, 559 bp, Physcomitrella patens
  CDS (3)..(443): 12_ck22_b09fwd
cgctgccgtcgcagtggtgttgtttcacattgctggaggtagttgcgcatcgtgcagggttgaggtacctgtcaaccatggtgggagctctcgggaagaatttcgcgactttcgcctctcgtttgagaggaggccatggctgtgacacagctgctgcggcagcggtttgggcggtgtctaagaggttcatgtcgtcttcgggcgaatcgattactgtccgagaggctctaaacagtgccatcgacgaggagatgtcagctgattccaaagtctttgtcatgggcgaagaggttggtgagtaccaaggtgcctacaaggtcacgaagggtctcttgcagaaatttggaccagatcgtgttttagatacccctattacagaggctggattcgcaggtcttggtgttggagcagcaatgtatggcctgaacctattgttgagtttatgacctttaacttcccatgcagccattgatcatctcatcaattcgctgctaaaacaaactacatgtctggcgggacaattaatgttcctatagtattcaggggtcctaatggtgca

SEQ ID NO: 78 - protein, 147 aa, Physcomitrella patens
LPSQWCCFTLLEVVAHRAGLRYLSTMVGALGKNFATFASRLRGGHGCDTAAAAAVWAVSKRFMSSSGESITVREALNSAIDEEMSADSKVFVMGEEVGEYQGAYKVTKGLLQKFGPDRVLDTPITEAGFAGLGVGAAMYGLNLLLSL

SEQ ID NO: 79 - DNA, 491 bp, Physcomitrella patens
  CDS (88)..(489): 37_mm3_g01rev
cccaattctcacattccatggaactgagcatcacagaactcggtgcatttgttttggaatgttgttgtggaaaacggtttctgtgttatgccaaaagttccggcaaccttcagtctttacagcgtagtgggcgtgcggagaatgttgttggagagttctgggagacccaaactaatgcgacaggggactcgctggatgtcaaaaacagtggacatgaaggagagattggctgaattaatcccgaaagaacaggaccgtttgaagaagattaagaaagattatggcaagatatctctcggagacaccactgtcgacatgtgcattggcggcatgcgtggcatcaaaggcatgctatgggagacatccttgctcgacgctgatgagggtattcggttcaggggactctccattcctgaatgccagaagaagttgccggcagcaattagtggaggcgagcctttgcccgaaggcttattgtggcttttagttac

SEQ ID NO: 80 - protein, 134 aa, Physcomitrella patens
MPKVPATFSLYSVVGVRRMLLESSGRPKLMRQGTRWMSKTVDMKERLAELIPKEQDRLKKIKKDYGKISLGDTTVDMCIGGMRGIKGMLWETSLLDADEGIRFRGLSIPECQKKLPAAISGGEPLPEGLLWLLV

SEQ ID NO: 81 - DNA, 625 bp, Physcomitrella patens
  CDS (107)..(625): 70_ppprot1_069_d11
gcacgagggtttttgtagccgtagaaactcgtttagtaactagagttattttgatttgttgggctacgttgggttccgaggagattgtcttgtacctcgaagagatatgacgatcggctcccctttggttgtagttggatcggtgaatgccgacatttatgtggaggttgagcgtctaccggccgagggtgaaaccatagctgctagaagtggccagactcttcctggtgggaagggagccaatcaagctgcttgtgccgctcgtctttcgtaccctaccttcttctgtggccaggtcggacaggacgcccatgccaacctggttagaaatgcacttgtttctgcaggcgttcatttagatcatgtaaatactgttgacgctccaactggtcacgcagttgtgatattacagccaggaggcaaaaactcaattatcatcgtcggaggagccaatgtcgcatggccgaagttagaagatggaataagcagtctaaccaccaacgcgcaggaattgattaaacgagcaggtgcagtgcttctgcaacgtgagattccagacgcagtcaatcttgaagctgcgaagattgctaagagtgccggtgttcctgtgatcatggatgctgga

SEQ ID NO: 82 - protein, 173 aa, Physcomitrella patens
MTIGSPLVVVGSVNADIYVEVERLPAEGETIAARSGQTLPGGKGANQAACAARLSYPTFFCGQVGQDAHANLVRNALVSAGVHLDHVNTVDAPTGHAVVILQPGGKNSIIIVGGANVAWPKLEDGISSLTTNAQELIKRAGAVLLQREIPDAVNLEAAKIAKSAGVPVIMDAG

SEQ ID NO: 83 - DNA, 589 bp, Physcomitrella patens
  CDS (2)..(250): 96_ck20_h12fwd
ggagatcagcgaggattctcctgccagacacagcttggtctgtcgcggcctcctgtcgctgctcgttgaggggtccgccaaggccaccgactcggaatccaccgacgctatcctcggcgcggctctagatcacgccttgaagcggaagctgtgcattgttggagactctgttgttgcgatccacagaatcggcgccgcgtctgttatcaagatcgtcgaggtcaaggagaaggtagtcaaggtggcggcatgaagacgaggggcgagtgcatcgtctcgactgcacccaaatccgtaattagtttataccaaggttgagcaattaatacaggaattattttcgccgtttatggcagcgtatggatgtattatagctcgttttagtaaatttcttctggaacatgcttcaacagcgggaacttgtactttctcctgctcttcgaggtaggatttgttgaagcgagtttcggagtattgagcgcgtccgaaaaatgttgaagatatgttcgatagcgtgctaacaatcgccacgatgatctttcagaaggaaaagcaatcgagaaaagaccagttcttacaaccaaaaaaa

SEQ ID NO: 84 - protein, 83 aa, Physcomitrella patens
EISEDSPARHSLVCRGLLSLLVEGSAKATDSESTDAILGAALDHALKRKLCIVGDSVVAIHRIGAASVIKIVEVKEKVVKVAA

SEQ ID NO: 85 - DNA, 386 bp, Physcomitrella patens
  CDS (9)..(386): 88_mm13_g11rev
gcggtgcaatgcagggagcctggggcggaggagccgcgggggcgctgctgccctgccagcgtctggagaggtcgcgttgcggccggatggaatgccgctactctggtgtacgctatggcaggagcgggacattggaacatgatcgttgtggggtcaggaaggcacatttcgaatcgacactgcagaacgtatcatggggacaagaccgcaagaagcctttgatcgtgagggcttcctcagagaaagtcagttttccagtatcagaatcgcgtgcagcgaatttgcgtaggttattagaacagccagggattcgtcaagcgccagcgtgttatgatgctttgagtgcgagccttgtggagaaagctggattcgacatcacttttatg

SEQ ID NO: 86 - protein, 126 aa, Physcomitrella patens
MQGAWGGGAAGALLPCQRLERSRCGRMECRYSGVRYGRSGTLEHDRCGVRKAHFESTLQNVSWGQDRKKPLIVRASSEKVSFPVSESRAANLRRLLEQPGIRQAPACYDALSASLVEKAGFDITFM

SEQ ID NO: 87 - DNA, 433 bp, Physcomitrella patens
  CDS (3)..(431): 18_ck25_c09fwd
gggccgtggtggtttgcaaagctgctgatgggcagactgtggtcatcggactggctgcggactctgggtgcgggaagtctacgttcatgcggaggctgacgtccgtgttcggaggcgctgcgactcccccgaagggtggcaaccctgactccaacacgctgattagtgacaccaccaccgtgatctgcctggacgactaccactccctcgacaggtatggtcgcaaggagaaggccgtgaccgcgttggaccccagggcgaacaactttgatctcatgtacgagcaggtgaaggccttgaaggagggcaagtctgttgagaagccgatttacaaccacgtgaccggtttgttggatgcccccgaaaccattcacccccctaagattctcgtcatcgagggtctgcacccaatgtacgacgagcgtgtccggga

SEQ ID NO: 88 - protein, 143 aa, Physcomitrella patens
AVVVCKAADGQTVVIGLAADSGCGKSTFMRRLTSVFGGAATPPKGGNPDSNTLISDTTTVICLDDYHSLDRYGRKEKAVTALDPRANNFDLMYEQVKALKEGKSVEKPIYNHVTGLLDAPETIHPPKILVIEGLHPMYDERVR

SEQ ID NO: 89 - DNA, 522 bp, Physcomitrella patens
  CDS (2)..(520): 83_ck30_f06fwd
ggcaggtgacctcgcagcaactgctgtgaacgcacccatggtacccgcggaggtaatcgccgagctcaccccgtatgtggttttggcagagaagctgggaaggctcactgtgcagttggtatcgggtagtgccggagtgaagcaggtgaaggtggtgtacaaatcttcccgggacgacggcgatttggacaccaggctattgagggccaggatctcgaaggggttgatcgagcccgtgtcggacgcaatcatcaatttggtgaatgcagattatgtggccaagcagcggggtttgaagattagtgaggagcgggagccagctgatggtgagagcggagtaccactggagagtgtgtccgttacaattcaagatgtagagtcaaaattcgccagcgcaagaggggatcacagccggagcatcaccttggagggcaaggtgaaggacggtgtgccccatcttagcaaggtcggaaacttcagtgtcgacgtgagcttggacggcaatgtcatcttgtacccggc

SEQ ID NO: 90 - protein, 173 aa, Physcomitrella patens
AGDLAATAVNAPMVPAEVIAELTPYVVLAEKLGRLTVQLVSGSAGVKQVKVVYKSSRDDGDLDTRLLRARISKGLIEPVSDAIINLVNADYVAKQRGLKISEEREPADGESGVPLESVSVTIQDVESKFASARGDHSRSITLEGKVKDGVPHLSKVGNFSVDVSLDGNVILYP

SEQ ID NO: 91 - DNA, 596 bp, Physcomitrella patens
  CDS (2)..(388): 63_ck7_c05fwd
cgcaagtgataagttctctcccgaagccaacactcaagtgtgcagctcctcaaacatccctgccggctggatgggactagacattggcccgaaggcaatcgaccaattccaggatgccctgaagggcgccaagacggttctgtggaacggaccgatgggagtgttcgagttcgagaagttcgcggacggaacaactgccgtcgctaaaactttggcaggtttgaccaaggagggtgccatcaccatcattggaggaggtgactccgtcgcagccgttgagaaggctggactcgccgaccagatgagccatgtgtccaccggaggaggggccagtctcgagttgttggaaggcaaggtattgccaggagttgctgctcttgacaacgcttaaatgcctcttttgccgaagaggtgaattccgtccacgaactcgcagtagagtgacaatgtcactgagtgcacccccggctctggctgtctaatcatatgattcatttttttggtagttttttttgtgatttgcctttgtaccagtaaacacagattatgaccagtaaagaactccagctcgatttacctggatgcttggtattctc

SEQ ID NO: 92 - protein, 129 aa, Physcomitrella patens
ASDKFSPEANTQVCSSSNIPAGWMGLDIGPKAIDQFQDALKGAKTVLWNGPMGVFEFEKFADGTTAVAKTLAGLTKEGAITIIGGGDSVAAVEKAGLADQMSHVSTGGGASLELLEGKVLPGVAALDNA

SEQ ID NO: 93 - DNA, 480 bp, Physcomitrella patens
  CDS (92)..(478): 18_ck24_c09fwd
ggaaacctcgatcatctccgcctgtgcagcagctgcagccgttctgttggagtgcttgtgtggtgccgcgcgcggtctttaagcttgaaggatggagtccctcgcgctgcgatctgccgtggttgccaccggattgacctccagtgtggcatcccaaacctctgtgcagacccgcgctacggtttcgtctgctttcatcgggaagagcatacgtgtgaacactaaactcaacgcctcagctgtgcccgtccagcagaagttccgctacgtgcgtgccgacgctggtgcacagactgcgcaggttgagaccgttgagaagaaggctagcattaaggacgttcccgagtcggaattccagggcaaggttgtgttcgtgcgtgctgatctgaacgtacctctcaatgatgcatgcgaaatcaccgatgacacccgaatccgtgcctccctcccgaccatccagcatcttacgaagccggagcc

SEQ ID NO: 94 - protein, 129 aa, Physcomitrella patens
MESLALRSAVVATGLTSSVASQTSVQTRATVSSAFIGKSIRVNTKLNASAVPVQQKFRYVRADAGAQTAQVETVEKKASIKDVPESEFQGKVVFVRADLNVPLNDACEITDDTRIRASLPTIQHLTKPE

SEQ ID NO: 95 - DNA, 444 bp, Physcomitrella patens
  CDS (200)..(442): 18_ck26_c09fwd
cctccactcacttttttcgttcctccacgccatcgttgagtgaataagcaacagttgggagtcggcagttgagcagcaggagttgagagctccttttgtctgagcggcagttgttgtccctggacacgcgatcgcagcgaacgcatcgagatcgttgcggttttgtttgcatttagaaatcatggtcgctgctgtcgggatgtcgcaggtctcctcctccggtattaaatggagcttctgtgggacttcgctggagaggaaggccgctgccgcttctaaggcgacgaatgtagcgttcgctgtgcgtgccgctggttatgacgaggagctcgtgaagaccgcgaaaacgatcgcgtcacccgggcgcgggatcttggccatcgacgagtcgaatgctacgtgtggcaagcggctggcctcggttgggctggaagaacaacgagg

SEQ ID NO: 96 - protein, 81 aa, Physcomitrella patens
MSQVSSSGIKWSFCGTSLERKAAAASKATNVAFAVRAAGYDEELVKTAKTIASPGRGILAIDESNATCGKRLASVGLEEQR

SEQ ID NO: 97 - DNA, 403 bp, Physcomitrella patens
  CDS (74)..(403): 60_ppgam17_b12
cgggacgactcccttgttagtttgtgttctgcctcctgattcatctctccaggctgcaatggctgccgtcggtatgaccatgtcgctcaggagcagcaccaccctcaagggtgaattctgtggcgcggctgtgaggcagagcgtcgccgcccccaaggctgccaatgtggcgttcgccgtgcgcgctggacagtatgacgatgagcttgttaagaccgcgaacaccattgccaccaagggtaagggtatcttggccatggacgagtccaatgccacctgcggcaagcgtctggagtcgatcggtttggagaacacggaagccaaccgtcaggcgtacaggcaattgttggtgaccacccccgatcttggtgactacatctctggagccatcctcttcgaggag

SEQ ID NO: 98 - protein, 110 aa, Physcomitrella patens
MTMSLRSSTTLKGEFCGAAVRQSVAAPKAANVAFAVRAGQYDDELVKTANTIATKGKGILAMDESNATCGKRLESIGLENTEANRQAYRQLLVTTPDLGDYISGAILFEE

SEQ ID NO: 99 - DNA, 228 bp, Physcomitrella patens
  CDS (2)..(169): 50_ck19_a10fwd
tgccggaggctacagtagtgatggcaagcaatctgttctggacaaggtggtggtgaacaccgacgacagaacccaggttgcatatgggtctcgcgatgagatcatccgcttcgaggaaacgttgtatggcgactccaggctcaaggctgagctcgccgctgccactgtgtagattgtttttgtatctctttttttgcactttttttaattttttttttattatttttt

SEQ ID NO: 100 - protein, 56 aa, Physcomitrella patens
AGGYSSDGKQSVLDKVVVNTDDRTQVAYGSRDEIIRFEETLYGDSRLKAELAAATV

SEQ ID NO: 101 - DNA, 574 bp, Physcomitrella patens
  CDS (65)..(574): 35_ck11_f03fwd
gtgattgtgtattgagagtcgaattccttcttgctttgtgcgttgtcgaggagctgaagcagctatggcgaccacacaagcgattctctctgccacccttgccatagccccggcttccagctgcgagacttcgtcacggagcccggcgtccaccaaaacttgtctctcagtggcaggatcgtcgctgcatggctcagtggccggactcggagctgggaaacagattgtgagcgtgcagaggaagagcgttgccgtgagggccgccgttgcagctgagactgccgctcccaagcagcaggcgaagagccagtatgacatcactaccctgacgacgtggttgctgaagaaagagcaggcgggcgtcatcgatggcgagctcaccattgtgctctccagcatcgccctggcttgcaagcaaattgcgtctctggtgcagagggctggcatctccaacatgactgggttgcaaggagctgctaacattcaaggggaggaccagaagaagctagacgttatttcgaacgaggtgttctcaagctgtctgcgctcaagcggacggacaggcatcatcgct

SEQ ID NO: 102 - protein, 170 aa, Physcomitrella patens
MATTQAILSATLAIAPASSCETSSRSPASTKTCLSVAGSSLHGSVAGLGAGKQIVSVQRKSVAVRAAVAAETAAPKQQAKSQYDITTLTTWLLKKEQAGVIDGELTIVLSSIALACKQIASLVQRAGISNMTGLQGAANIQGEDQKKLDVISNEVFSSCLRSSGRTGIIA

SEQ ID NO: 103 - DNA, 568 bp, Physcomitrella patens
  CDS (3)..(245): 20_ppprot1_083_d07
caatacaaaggttagagccagtgattattgaacttgagagacagcgttcgcctgtagttgtcattgcgcatcaagcaattctacgctctctatatgcgtactttgcagacaaacctctgaaagaggtaccgcatattgagatgccactgcatactattattaagattcaaatgggagttactggtgttcaggagaagcgatacaagctcatggagacccagagcaccaaatacggtgaattcccatgagtatggcagtcctaagcagttttctctagcaatttacgactccaacgacgagctttcaaacaagccatgtagttttgaggaccataggaaatgcttgataaacttaatgaaatcacagcaacctgctattcgaattttattttgtgcagtaattacataggaaatcatttcattactgaacttgattttacatcatggctttctatattctagtatttactttgcgtgcactccgcactatttcgtgggatagaatcagtattcttttgtatcattattctagaaatttaagtaatgcaaaacatgcgtttaaaaaaaaa

SEQ ID NO: 104 - protein, 81 aa, Physcomitrella patens
IQRLEPVIIELERQRSPVVVIAHQAILRSLYAYFADKPLKEVPHIEMPLHTIIKIQMGVTGVQEKRYKLMETQSTKYGEFP

SEQ ID NO: 105 - DNA, 182 bp, Physcomitrella patens
  CDS (3)..(182): 14_ck4_c07fwd
agtaccgggagcttgcgcatagaatcgacgaggctttgggcttcatgtccgcgtgcgggctcaccctggatcacccaatcatgacctccactcaattctggacctctcacgaatgcctgctgctcccatacgagcaagccctcacccgagaggattctacctcgggattatggtacgtctgc

SEQ ID NO: 106 - protein, 60 aa, Physcomitrella patens
YRELAHRIDEALGFMSACGLTLDHPIMTSTQFWTSHECLLLPYEQALTREDSTSGLWYVC

SEQ ID NO: 107 - DNA, 548 bp, Physcomitrella patens
  CDS (2)..(424): 89_ck12_g06fwd
caaggttcggctcgcaggaaaccccaatgtaatggtgtgcgagagaggcaccatgttcggctacaatgacctaattgtggatccccggaacttggagtggatgcgagaagcgggtgctcccgttgttgcagatatcactcattctctacaacagccggctgggcaacagctggagggaggaggcgttgcgagcggtggcctgcgcgagctcatcccctgcattgcccgggcttgtgtggccgtaggagtagacggcatcttcatggaggtgcacgacaaccccaggcaggcgccctgcgatggccctacgcaatggcccttgcgtaaccttaagaacctgctggaggagctcatcgctatcgccaaggttaccaaggggaaggagaggatggcgattgacctcactcctgtcgacgaagacttctagcttctacgaacccaattgagttctcagctttgcaatcacacttggaggtggcacttgcaaggtttactccacatatgcattccactcgatttggagtcttgagtgcagccattgagcaaca

SEQ ID NO: 108 - protein, 141 aa, Physcomitrella patens
KVRLAGNPNVMVCERGTMFGYNDLIVDPRNLEWMREAGAPVVADITHSLQQPAGQQLEGGGVASGGLRELIPCIARACVAVGVDGIFMEVHDNPRQAPCDGPTQWPLRNLKNLLEELIAIAKVTKGKERMAIDLTPVDEDF

SEQ ID NO: 109 - DNA, 343 bp, Physcomitrella patens
  CDS (76)..(342): 55_bd01_b04rev
ccgccccagtatttagtgtgcgagcaaatttgaattgcgggggtcctgaagtctgttgtaaagttgcaaggagcgatggtgacgatgacaatgacaggggctgccttgggagggtgcacggcgaaaattactgcgcagagcagcttctggggcgaaaggcagcgctctgtcaaggtctccgccaccacgagtggaagggcggcgatgccagcaattgaggcaactcaccgtgttgataagttctcaaagaatgacatcatcgtttcgccctctatcctatcagctaacttcgccaagttgggagagcagatcaaggctgtggaaaacgccggatgcgactgga

SEQ ID NO: 110 - protein, 89 aa, Physcomitrella patens
MVTMTMTGAALGGCTAKITAQSSFWGERQRSVKVSATTSGRAAMPAIEATHRVDKFSKNDIIVSPSILSANFAKLGEQIKAVENAGCDW

SEQ ID NO: 111 - DNA, 576 bp, Physcomitrella patens; identical to SEQ ID NO: 73
  CDS (1)..(576): 50_mm15_a10rev

SEQ ID NO: 112 - protein, 192 aa, Physcomitrella patens; identical to SEQ ID NO: 74

SEQ ID NO: 113 - DNA, 454 bp, Physcomitrella patens
  CDS (239)..(454): 86_ck23_g10fwd
attgcatagtgtccccgcagcagcagcagctatggcgtccaccatgttggagtctcatctggtctctagcgtcacattgcgggtagcagcagcacctcgcaattcggcagcctcggcctcggcctctgccacagcgcaggctgtgccttgcaaggcactgggagcgtcgtcatctacgttgcttcgtggcgggcaactgaggacgggtttcgtggctgtgcacagaaggccttgcacgatggccttccgtccccgtgcgcaatcgcaaggtaccaagcttacacaggatgaactcaagaagattgctgccgagaaggctgtggaatatgtcaagagtggcatggtcttgggtttgggcacaggttctacagctgcatttgcagtggcaaagattggcgagcttttgaaggaaggaaagctgacggatattgttggagtgcctacctcaaagaga

SEQ ID NO: 114 - protein, 72 aa, Physcomitrella patens
MAFRPRAQSQGTKLTQDELKKIAAEKAVEYVKSGMVLGLGTGSTAAFAVAKIGELLKEGKLTDIVGVPTSKR

SEQ ID NO: 115 - DNA, 341 bp, Physcomitrella patens
  CDS (1)..(336): 22_ppgam15_d08
cagaaaataaatccttggatttaccagatcattagagtgtacaagaataaggaacatgctgaagttgaattcactgtgggtcccatacctattgaagatggagttgggaaagagattgtcactcagatctcaaccaccataaatagtaacaaaagattttactctgattctaacggccgagacttcattgaacggattcgagactacagagctgactgggatcttgaagtgaatcaaccaattgcaggaaattattatcctataaatcttggaatttacctgaaagatgataacaatgagttctcgatcttggtacatagatctgttggtggatcccatgt

SEQ ID NO: 116 - protein, 112 aa, Physcomitrella patens
QKINPWIYQIIRVYKNKEHAEVEFTVGPIPIEDGVGKEIVTQISTTINSNKRFYSDSNGRDFIERIRDYRADWDLEVNQPIAGNYYPINLGIYLKDDNNEFSILVHRSVGGS

SEQ ID NO: 117 - DNA, 467 bp, Physcomitrella patens
  CDS (2)..(466): 70_ck11_d11fwd
gctgaagttggttgagccctgcgccgcgatgacgaaggacctctacttcaggtccatcgtccccatcggcctcctcttctcgctctctctgtggttctcgaattcggcttacatctaccttagcgtctccttcatccagatgctcaaggcgctcatgccggtggcagtctactctcttggggtacttttcaagaaggatgtattcaactcttcgaccatggctaacatggtcatgatctccattggtgtcgccattgcggcctacggggaggcgcggttcaatgtctggggtgtcacgctgcagcttgcggctgtatgcgtggaagccctccgtcttgtcttgatccaaattcttctcaactcccggggaatttccctcaatcccattacaacactctattacgtcgcgcccgcgtgttttgtcttcctctctgtcccttggtatctcatcgaatggccgaagctgc

SEQ ID NO: 118 - protein, 155 aa, Physcomitrella patens
LKLVEPCAAMTKDLYFRSIVPIGLLFSLSLWFSNSAYIYLSVSFIQMLKALMPVAVYSLGVLFKKDVFNSSTMANMVMISIGVAIAAYGEARFNVWGVTLQLAAVCVEALRLVLIQILLNSRGISLNPITTLYYVAPACFVFLSVPWYLIEWPKL

SEQ ID NO: 119 - DNA, 591 bp, Physcomitrella patens
  CDS (3)..(284): 29_ck12_e03fwd
tggcgagtttcttgttgggatggggaatcacgatcggagcgggtctggcgtcgtaccccatcgacacggttcggcgtaggatgatgatgacctccggagaggcagtgaagtacaacgggtcgatggacgcgttcaagcagattttggcgaaggagggagcgaagtcgttgttcaagggcgctggtgcgaacatccttcgtgcggtggctggagccggagtgttgtcgggatacgatcagttgcagatcttgcttctgggcaaggcctactctggaggcagcggctgagtgcttcgtagcggattatgaagagaattttgttgccctggtcaatctttaatttagcactttcttttttgtagtgtaactttttgagttctttcgcgttctgactatcatagtgcacatgcgtatagtgcgctgagctggttatcgtgttagttttgtgtcttcgaattaacttcgagattactcaacttaggcgccataatgtgcttatttacaactcttatgaagcaagaattgctggcttctggttcgtccattgtgctctctttttatcttcatttaatatcattctatacacctcaac

SEQ ID NO: 120 - protein, 94 aa, Physcomitrella patens
ASFLLGWGITIGAGLASYPIDTVRRRMMMTSGEAVKYNGSMDAFKQILAKEGAKSLFKGAGANILRAVAGAGVLSGYDQLQILLLGKAYSGGSG

SEQ ID NO: 121 - DNA, 527 bp, Physcomitrella patens
  CDS (3)..(161): 07_ppprot1_057_b01
cttgggacgagctctttggcgggggcaacatgccggcctttttgtttggagctgtggctgcttttatcgggggcatcgcagccgtgcttctcctacctcgccctccgcccgattttaccaccaggaaccggcttcgcaggactcacagttcccctattccttgaacttgtagagtttatcgttcctttgaattgtagtgccttcgttttcgtgtagaaggtcataaattatggcaggttgcagtgatttttagacttcgaattttatgaatcagtctgtgataaggtcttcgatccgccagagtgttgattggtatattgtgtatgataaagtaggtgatgttcacatgtgatatggtggttaagggagcaactaagtcgcagtgctgctgaaacccaaatcttagagttttgttcgaagtctatcggttcaaattcaaatggactaaataccgtgctgcaatcaatgctaggtcggttttttttcgtatgctgaaaatagaattatcctgttttaaaattgttaac

SEQ ID NO: 122 - protein, 53 aa, Physcomitrella patens
WDELFGGGNMPAFLFGAVAAFIGGIAAVLLLPRPPPDFTTRNRLRRTHSSPIP

SEQ ID NO: 123 - DNA, 613 bp, Physcomitrella patens
  CDS (1)..(210): 25_ppprot1_057_e01
aacctcgcagtcgtcgcaccccaaatggttgtgtcggtggggagtgggccttgggacgagctctttggcgggggcaacatgccggcctttttgtttggagctgtggctgcttttatcggtggcatcgcagccgtgcttctcctacctcgccctccgcccgattttaccaccaggaaccggcttcgcaggactcacagttcccctattccttgaacttgtagagtttatcgttcctttgaattgtagtgccttcgttttcctgtagaaggtcataaattatggcaggttgcagtgatttttagacttcgaattttatgaatcagtctgtgataaggtcttcgatccgccagagtgttgattggtatattgtgtatgataaagtaggtgatgttcacatgtgatatggtggttaagggagcaactaagtcgcagtgctgctgaaacccaaatcttagagttttgttcgaagtctatcggttcaaattcaaatggactaaataccgtgctgcaatcaatgctaggtcggttttttttcgtatgctgaaaatagaattatcctgttttaaaattgttaactactaactgacagttcaatgaaaacgcccatttccgc

SEQ ID NO: 124 - protein, 70 aa, Physcomitrella patens
NLAVVAPQMVVSVGSGPWDELFGGGNMPAFLFGAVAAFIGGIAAVLLLPRPPPDFTTRNRLRRTHSSPIP

SEQ ID NO: 125 - DNA, 521 bp, Physcomitrella patens
  CDS (1)..(519): 48_ck24_h09fwd
cgcattcagaagcgggctacatcttccgtgcgcgcccaagctgctgatggagaagcctcgggggatgttgccactagacaatctaatcctgctaccactggaatggtcttgcctgcagttggtattgcctgccttggggcaatcttgtttggttaccatctcggggtggttaatggtgcattggagtacatttctaaggatctagggtttgccacggatgctgtaaaacaaggatgggtggtaagctcaactctagctggtgccactgtgggttcctttactggaggcgcccttgctgacaacttaggtcgcaagcgtacattccagattaacgccgtgcctcttattgtgggcactcttctcagtgcaaaagcaaccagtttcgaggctatggtgattggaagaattttggttggtgttgggattggagtttcatctggtgttgtgcctctatacatttcggaggtctcgcccacagagattcgaggtaccatggggacattgaatcagctctttatttg

SEQ ID NO: 126 - protein, 173 aa, Physcomitrella patens
RIQKRATSSVRAQAADGEASGDVATRQSNPATTGMVLPAVGIACLGAILFGYHLGVVNGALEYISKDLGFATDAVKQGWVVSSTLAGATVGSFTGGALADNLGRKRTFQINAVPLIVGTLLSAKATSFEAMVIGRILVGVGIGVSSGVVPLYISEVSPTEIRGTMGTLNQLFI

SEQ ID NO: 127 - DNA, 638 bp, Physcomitrella patens
  CDS (1)..(465): 41_ppprot1_105_g03
cctgtgtatcagcgtgcaggcaccatcattccaaagaagctaaggcatcgtcgcagctccactcaaatgctgaatgacccatacactttggttgtggctttggactccaactacgaggctgaaggtgagctctacatcgacgacggcaaaacctatgagttcgaaaagggcgctttcattcacagacgtttcaagtttgtcaaggggaaactaacctccactaacttggcaccctcgaagtccaacccgaagaagttcacgtcaccttgccttgttgagcgcattgttatcatgggagttcgagccagggatttggtcactggtaaaggcgcagtcgttgagggtgaaaggtggattcagaccgatattggagctccgtcattgatacctggtgcccattcaagcgctttgattctcaggcttccaaatgtgcgcattgctgatgactggtctattaaacttggttaaggatttggtaggagattatccaaaatatagtggggtgcatcaaccaaattcagtttttagtgcatatagggtgatggaaattagaagcatagtgcctactggatttagcatggaacaggtcagacttgacgaagtgattgttgtttattttctgaatccattatcagttg

SEQ ID NO: 128 - protein, 155 aa, Physcomitrella patens
PVYQRAGTIIPKKLRHRRSSTQMLNDPYTLVVALDSNYEAEGELYIDDGKTYEFEKGAFIHRRFKFVKGKLTSTNLAPSKSNPKKFTSPCLVERIVIMGVRARDLVTGKGAVVEGERWIQTDIGAPSLIPGAHSSALILRLPNVRIADDWSIKLG

SEQ ID NO: 129 - DNA, 599 bp, Physcomitrella patens
  CDS (1)..(597): 44_ppprotl_075_h07
caacgttgtgagattgatttgaggaaagaagcggttatcaggattgcagctgaagcgccatatcccgtgataacattcgggccgtatcccaacccggaggcgttgttagttgcgcttgcgagtgctattggcaccatccaaatgcctccgaagtgggctcttggataccaacaatgtagatggagttacgaaacggcagagaaagtatctaagattgctaatactttccggcagaagaacataccttgtgatgttgtctggatggatatcgattacatgcatgggttcaagtgctttacatttgatgagaacttcttcccagatccgaaggctctttcagacgagttgcattccattggctttaaggggatctggatgcttgatcccgggatcaaggcagagaaaggttgggacgtttatgacagtggaactgaggtggacgcttggattcaaacttcgaatgggaaggattttattggtgaatgttggcctgggttagtcgtgttcccagatttcacaaacaaaaacacccgcaagtggtggtccaaattggtggaaaagttcgttgctaatggtgtggatggtatttggaacgatat

SEQ ID NO: 130 - protein, 199 aa, Physcomitrella patens
QRCEIDLRKEAVIRIAAEAPYPVITFGPYPNPEALLVALASAIGTIQMPPKWALGYQQCRWSYETAEKVSKIANTFRQKNIPCDVVWMDIDYMHGFKCFTFDENFFPDPKALSDELHSIGFKGIWMLDPGIKAEKGWDVYDSGTEVDAWIQTSNGKDFIGECWPGLVVFPDFTNKNTRKWWSKLVEKFVANGVDGIWND

SEQ ID NO: 131 - DNA, 727 bp, Physcomitrella patens
  CDS (3)..(707): 63_ppprotl_60
tttcggagttctacaaggtgatgccctttgacggactgtggctggacatgaacgagccttccaatttctgttctggaccgaattgctactatcctcccgacgtcgtatgcccggaagcgctcgactggtgctgcatggtttgcgacaacacgaatgtctcgcggtgggacaggccgccataccgcatcaccaacacatggaacaaggagctttacgagaagaccgtcactatgactgcacgccactacaacgacgtcaagcactacgatgcgcacaacatctacggattcagtcagacggtcgccacttttaaagctctcaaagaggtaaccaagaagcggccatttgtgatgtcacgctccttgtatcctggctcgggtgcctccgctgcgcactggtcgggtgacaacggcgcttcatggaacgatttacgatactcgatcgccagtatcttaaattcaggcctgtttggcattcctatggtaggggcagacatctgcggcttcatcccagccacttgggaggagctttgcaatcgatggattcaggtgggcgccttctatcccttcgctcgcgaccactctgacgtgcacttcggcccccaggagctctacctctggaaatccgtcacacattccgcgaggaaagtcttgccactgcggtataaactcctcctttcatgtatacgctactccacgaagctcacatgacgggcgctcccgttgca

SEQ ID NO: 132 - protein, 235 aa, Physcomitrella patens
SEFYKVMPFDGLWLDMNEPSNFCSGPNCYYPPDVVCPEALDWCCMVCDNTNVSRWDRPPYRITNTWNKELYEKTVTMTARHYNDVKHYDAHNIYGFSQTVATFKALKEVTKKRPFVMSRSLYPGSGASAAHWSGDNGASWNDLRYSIASILNSGLFGIPMVGADICGFIPATWEELCNRWIQVGAFYPFARDHSDVHFGPQELYLWKSVTHSARKVLPLRYKLLLSCIRYSTKLT

SEQ ID NO: 133 - DNA, 599 bp, Physcomitrella patens
  CDS (2)..(565): 74_ck13_el0fwd
caaagacttcactctcgatcccgtcaattatcccgttgacaaattactgccctttgttcagaacttgcacaagaaccatcagaagtttatcatgattctggaccctgggatcaagatcgataccaactactccacctatgttcggggtgacaaactggacatattcatgaggaatggtacgagccaccgctacgttgcccaggtatggccaggcgccacaaacatacccgacttcctccatcccaaatcgcaggagttttggtcaacagaggtagccgaatttcataaagtgattccatttgatggcttgtggcttgacatgaacgagcctgccaacttctgcggtggtcctacttgttatttcccccccggaattcagacttgtcctcagatcgatgagtgttgcatgatatgtgataacacaaatctcaaccggtgggatgatcctccataccacattaactctcttggtatccaccggcctttgtacgcgcacacaatgggcgatgaactgcgagcatttcaatgggtatccgggcttacgatacccacaacgtctatggaatgagcgagggacttgcgacataccgacgcttaag

SEQ ID NO: 134 - protein, 188 aa, Physcomitrella patens
KDFTLDPVNYPVDKLLPFVQNLHKNHQKFIMILDPGIKIDTNYSTYVRGDKLDIFMRNGTSHRYVAQVWPGATNIPDFLHPKSQEFWSTEVAEFHKVIPFDGLWLDMNEPANFCGGPTCYFPPGIQTCPQIDECCMICDNTNLNRWDDPPYHINSLGIHRPLYAHTMGDELRAFQWVSGLTIPTTSME

SEQ ID NO: 135 - DNA, 607 bp, Physcomitrella patens
  CDS (1)..(318): 03_ppprot1_056_a02
cagggttatgcttacatcctcactcatcctggtcagccttgtgtgttttacgatcatttgtatgagtggagcggcgacttgaagagggttattctagaattgattgatattcgcaggaaacttgaggttcatagtcgatcgcacatcacgatacttgaagcagacacgaatggttattcagccgttgttgacaacaagctgtgcgtgcgattaggcaacactgaatggacccctccctctgatagtctctgggaacttactctctcgggcagtggctatatgatatggagtaaaccccagccgctcttaacctctcaatgagcgtcttgctcgtgaatttcagacgcattcagcatttatttattatacatgaattgtccattgtaaagtggataaggatcccgtagcgatctacattaatttcttatgattggcttgcgaaacaatcactagttgccacagcagtcaccttgacttgtgtgatgaattagtattttcaaacacttttacatatttgaatagtttcctgaagccactgcattgtggtcgggttatctgatagttggaatttaacaattattgattttataaaatacatatacgattt

SEQ ID NO: 136 - protein, 106 aa, Physcomitrella patens
QGYAYILTHPGQPCVFYDHLYEWSGDLKRVILELIDIRRKLEVHSRSHITILEADTNGYSAVVDNKLCVRLGNTEWTPPSDSLWELTLSGSGYMIWSKPQPLLTSQ

SEQ ID NO: 137 - DNA, 602 bp, Physcomitrella patens
  CDS (2)..(601): 50_ck1_a10fwd
gaagagctcgtgctggtatgatgtcatgggcgagactgcggaagatctagcagcagcaggcattactgatgtctggtttcctccttctagccattccgtgtccccccagggatacatgccaggaaggctttacgatttgaacgactgtaaatatggcaatgaagagaagctgagggaaaccattgaaaagtttcacagagtgggagttcggtgcattgctgatattgtcgtgaatcatagatgcggtgaagaacaagacgagaggggtgaatgggttatttttgaaggaggaacgcccgatgatgctctcgactggggtccttgggctatagtcggagatgactatccctatggtaacggaacaggtgctcccgacaccggagatgactttgaggctgcacccgacattgatcacacaaacgatatcgttcaaagcgaccttatcgtctggatgaattggatgaagttcaaaattgggtttgatgggtggagattcgactttgccaagggttacggtggatacttcgtcggtcgctacatcagaaaaactgaaccacagtttgcagttggggagttctggacgagcttgaattacggacatg

SEQ ID NO: 138 - protein, 200 aa, Physcomitrella patens
KSSCWYDVMGETAEDLAAAGITDVWFPPSSHSVSPQGYMPGRLYDLNDCKYGNEEKLRETIEKFHRVGVRCIADIVVNHRCGEEQDERGEWVIFEGGTPDDALDWGPWAIVGDDYPYGNGTGAPDTGDDFEAAPDIDHTNDIVQSDLIVWMNWMKFKIGFDGWRFDFAKGYGGYFVGRYIRKTEPQFAVGEFWTSLNYGH

SEQ ID NO: 139 - DNA, 550 bp, Physcomitrella patens
  CDS (2)..(550): 25_ppprot1_104_e01
tttggacaccgtaagcatgaataacactctgaacagacgtcgcgccttggacgcatctttgctggctctgaaatcggctggtgtggagggggttatgatggatgtttggtggggaatcgtcgagaaagatggccctcagcagtacaattggtctgcgtatcaagagttaattgatatggtgcggaagcatggtttgaaggttcaggctgtgatgtcctttcaccagtgtggtggcaacgttggcgacagttgcaatattcctctgcctccatgggtgttggaagaggtacgaaagaatccagacttggcctacaccgataaggctggaaggcgcaactcagaatacatctctcttggcgctgacaacgtgcccgctttgaagggaaggacaccggttcaatgctatgcggatttcatgaggagcttcagagacaacttcgacgattttttgggagattttattgtcgaaatccaatgcggaatgggacccgctggtgaacttcgttacccttcataccctgagagtgagggtaggtggcgttttccaggc

SEQ ID NO: 140 - protein, 183 aa, Physcomitrella patens
LDTVSMNNTLNRRRALDASLLALKSAGVEGVMMDVWWGIVEKDGPQQYNWSAYQELIDMVRKHGLKVQAVMSFHQCGGNVGDSCNIPLPPWVLEEVRKNPDLAYTDKAGRRNSEYISLGADNVPALKGRTPVQCYADFMRSFRDNFDDFLGDFIVEIQCGMGPAGELRYPSYPESEGRWRFPG

SEQ ID NO: 141 - DNA, 655 bp, Physcomitrella patens
  CDS (3)..(215): 53_ppprot1_074_a06
ctttgttgtcggaaggaaaggtacctttgggcgtcggcgagaactcaaagctgaggaattgtattgtggacaagaatgcaagaattggcaaggatgttgtcattgcgaacactgacaatgtcttggaagcggagagacaaagtgaaggtttttacatccgttccggaattgtagtagtatacaagaacgcggttatcaagcacggaactgtaatctaaattacgaatctttctccatatcgtgaaaactgcttccttgcaacgccggtccctggtgagctgatccttggacctcatcatcgacggtgaaaaagatatcagtacttatccgagttgtgagattctcagctcgtgattactgatcctcttcctcttgccaccctgaagattgccgcgcaggatctggtttgtgtcggtatatcagatcagtagttcttacataagactgaattgaaatgtacaaagagatcagatcatcgggagtggaaccgttcagaggaagaaacctaccgtatagtcaagtggacgaagattatggtatcaattttggagtgtaaagtgtgagcgacttctactgtccttgtatcataccccattgaagtaaagaattgtgcaaagccttgacagcgatgctgttgatgtctgatgcttcactc

SEQ ID NO: 142 - protein, 71 aa, Physcomitrella patens
LLSEGKVPLGVGENSKLRNCIVDKNARIGKDVVIANTDNVLEAERQSEGFYIRSGIVVVYKNAVIKHGTVI

SEQ ID NO: 143 - DNA, 554 bp, Physcomitrella patens
  CDS (2)..(553): s_pp001047038r
gcacgagggtctggaaaatccaccaggagcacattgttgtttgcctcacgtagaaaatctgctggacgctcgtagtcccatggccgcccaagtatttttcaatggcagtttgctgggaggcattagttgcacttcaacacccagttcactttctgtgcctcgatcttcgctgacactacctgtgccaacttccttgcgcaagagtctttactctatggtaagctggaaggatttgctttctaggcaccgttcttttaagaggggtatgaggtcaagcgctttagctgattcagacttttcagtgaagaaagatgagatgcaaacgcaaagtatgtttccagctgcaagacgtggcaaggagaatgcggtttctacgaccactgtgaacgctacaagtgtacttccggaacgcaaaatcattcatgagacagcggtagttcatccggacgccttcataggtgagggggttgtcatcagcgcattttgtacagtgggacctggtgtttcaataggaaatggctgcaagttacatcctagtagtcacgtctgtggga

SEQ ID NO: 144 - protein, 184 aa, Physcomitrella patens
HEGLENPPGAHCCLPHVENLLDARSPMAAQVFFNGSLLGGISCTSTPSSLSVPRSSLTLPVPTSLRKSLYSMVSWKDLLSRHRSFKRGMRSSALADSDFSVKKDEMQTQSMFPAARRGKENAVSTTTVNATSVLPERKIIHETAVVHPDAFIGEGVVISAFCTVGPGVSIGNGCKLHPSSHVCG

SEQ ID NO: 145 - DNA, 1660 bp, Physcomitrella patens
  CDS (224)..(1270): c_pp030002055r
ttggcgcgccaggacaggcagaggacgagacaagggggatagcgttgcgacttcgtttgtttcccactctggagcactggaatcacgtcgtgtatctgagaccgagattggcagaggcgatcgccagacgtttgtcaatctcgactgctctctctgctcgtggtttcagttcgcgaggtgacatcgtaagcaaattgtgcggtctcggagcagattgtttgcaatgtctcaagaggcggcggcttcgaagagggcgctcatcactggtatcaccggccaagatggctcgtacttgaccgaattcttgttgaacaagggttatgaagtgcatgggatcatcagaagatcctcgaacttcaacactcagcgtttggagcacatctacatcgatccgcaccagtccagcgctcgcatgaaactgcactacggagatctgtcacacgcgtcgtcattgcggaaatgggttgactcgatccgcccggatgaggtttacaatctgggggcgcagtctcatgtgggagtgtcgttggagaatccggattatacctccgatgtggtaggcaccggaacgctaaggctgttggaagctattcgaattcacatccaagccaccggaaggctggtgaagtactaccaagctggatcttctgagatgtacggtgccactcctccgcctcaagacgagaccaccgtgttccatcctcgcagcccttacgccgtcgccaaggtggcagggcatttctacaccgtgaactaccgagaggcatacgggatgtttgcatgcaacggcatcctgttcaaccacgagtctccccgccgaggagagaacttcgtgacgaggaagatcactagagccattggtcgcatcaaggtgggcctgcagaagaagctgtatctaggtaacttgaaggcgtctcgtgattggggatttgccggagactacgtggagggaatgtggatgatgttacagcaggagaagcccgacgactacgtgctcgcgacggaggattcccacactgtggaggagtttctggaagaggcattcagctatgttggtctgaactggaaggaccatgtcgagattgatcccagatatttccgtccttcggaggtggacattctgcgaggcagtgcgcagaaagcgaaggaggtgctgggatggcagcctaaggtgcagttcaagcagctggtggcgatgatggtggatggtgatttggagaaggcgaagcgagagaaggtgcttgtggatgctggcttcattgactcgcaccagcagccctgaattttgggcaccgaatgaatagtgttaataattatatgaaacgaatggatataatatgacaggccttgcaatatatggttaatatattgatacatagtgatatgtcaacccgagagtacttcttcaattaggttatagccttagctttgccatgtaaggcttacaatatattcttcgctgccgcagtgcttagcacacaccaagtactagttccgagcaattttagtgggttgtttattcagcagaatgcactgacaccactcatctagaatataagcccgcattcgggtgcaaatcaatgctattctctgatgaggacgattttgccaacctgtgcaccctccttcgaaatgaatattcaattcttaaaactcgtgc

SEQ ID NO: 146 - protein, 349 aa, Physcomitrella patens
MSQEAAASKRALITGITGQDGSYLTEFLLNKGYEVHGIIRRSSNFNTQRLEHIYIDPHQSSARMKLHYGDLSHASSLRKWVDSIRPDEVYNLGAQSHVGVSLENPDYTSDVVGTGTLRLLEAIRIHIQATGRLVKYYQAGSSEMYGATPPPQDETTVFHPRSPYAVAKVAGHFYTVNYREAYGMFACNGILFNHESPRRGENFVTRKITRAIGRIKVGLQKKLYLGNLKASRDWGFAGDYVEGMWMMLQQEKPDDYVLATEDSHTVEEFLEEAFSYVGLNWKDHVEIDPRYFRPSEVDILRGSAQKAKEVLGWQPKVQFKQLVAMMVDGDLEKAKREKVLVDAGFIDSH

SEQ ID NO: 147 - DNA, 1490 bp, Physcomitrella patens
  CDS (347)..(1276): c_pp001064043r
aggacaggcagaggacgagacaagggggaccctcggtaagatttgacaggcagatctggctttgaaagaagcgagtctgcattggagtttcaggctgcgattttgtcggctggggattgttttgcgagattgtgtgagtcgacatgtattggatctggtggacagggttgagggttgtggtttctgcttggtctattagcagtgggtttgggagcctttgcattgccgtttgtgagttgggttttggcgcattttcttcagatatgaatctgtagtacaaatcaccaatcacacgttcaagaaaggtatttggcgtgtttgcagcacatctgtgcagatcgccagcatgggtgtcgacaaggacgccaagatctttgttgctggacaccgaggtctagtaggtgcggctgttgttcgtgctttgaagaaggatggttataacaatttggtgatgaagactcataaagaactagatcttacccgtcagcacgaggaatttttcgacacggagaaaccagcgtacgtcatcctagcagctgcgaaggtgggaggcattcacgcaaacagtacttaccctgcagagttcattgccgtgaatctgcagatccaaacgaatgtcatcgatgctgcttacaagtctggggtgaagaagctcttgtttctgggctcttcgtgtatctacccaaagtttgcccaggtacccatcgttgaggagtcgctcctgacagggcctttggaagctacaaacgagtggtatgctgtagcaaagattgcaggaatcaaaatgtgccaggcttacaggctgcagtataatttcgacgccatttctggaatgccgacaaacctctacggtccccacgacaatttccatcccgagaactcccacgtcttgccagccttgatcagacgctttcacgaggctaaggtgaacggcgctaaggaagtggttgtgtggggatcaggttccccattccgtgagtttcttcacgtggacgacttggcagaggcaacagtatttctgctgcagaattactccgcgcatgagcatgtcaacatgggcagtggctctgaggtctcaatcaaggaactcgccgaaatggtgaaggaagtggttggatttcaggggcagctgacatgggatacttctaagcctgatggaactccacgaaagctcatcgatagcagcaaacttgccaacatggggtggcaagcgagaattcccctcaaggaaggattggcagagacttacaaatggtactgtgagaactacaatgtctaggctattttattcggatcaaccttgaagcacctgtttttgaattcttactacgatagataaattcaagcggtggctatgtgaagcagtggtagctttgcaggatactgacctcgaggatatttatcacaattcattgcctgtttagtgggtactgcaaccttgtattgtgaggctgtcatggcaattttctttctagcatgctgactttaaa

SEQ ID NO: 148 - protein, 310 aa, Physcomitrella patens
MGVDKDAKIFVAGHRGLVGAAVVRALKKDGYNNLVMKTHKELDLTRQHEEFFDTEKPAYVILAAAKVGGIHANSTYPAEFIAVNLQIQTNVIDAAYKSGVKKLLFLGSSCIYPKFAQVPIVEESLLTGPLEATNEWYAVAKIAGIKMCQAYRLQYNFDAISGMPTNLYGPHDNFHPENSHVLPALIRRFHEAKVNGAKEVVVWGSGSPFREFLHVDDLAEATVFLLQNYSAHEHVNMGSGSEVSIKELAEMVKEVVGFQGQLTWDTSKPDGTPRKLIDSSKLANMGWQARIPLKEGLAETYKWYCENYNV

SEQ ID NO: 149 - DNA, 924 bp, Physcomitrella patens
  CDS (322)..(921): c_pp032009028r
attcatcagccggtctgccgtagcttcggcgcgcaggacaggcagaggacgagacaagggggctgcaggcaccaggctgcttccgcagctttagattgcaagagcaggttccctcaggacttcgaatctggatcgcgctcacagaaagtccacatgttagttgcctctcgtagtcgcgctgcttagtttcgacaggtttcaggctcctggaagtcttttgcaaccaggtttccgggccagcttgaacagcacttgttcggtactgtttagaagttgaactttgaagtgcgcaacgagatagtatttcgagaagtatcgacaatgggttccttgggaagacaaggatgtttattggtcggtgtcttgttttacttgagcatggctatcggcgctcaagctcagagttacccaggacttcaggctgcattcaattcttggacgccgaagcagattatcccggataagaatggaaggaaagtgcaactcgtgcttaacaattcatcttcggcatatactggcatgggatctaagcaatcgtggctgtttgggggtatcggggcctggatcaagctccccgctaacgattccgctggaactgtcaccacattctacatgtcatctactgggccgaagcattgcgagttcgacttcgagttcctaggcaactccagcggccaaccttaccttctccataccaacatcttcgtcgacggcgtcggaggccgcgagcagcagatccgcctatggtttgaccccactgcagcattccactactacaacttccagtggaacaacgacgtgctagtgttcttcattgacaacacagccatcgcatgttcagaacctagaaggcatcgtgccatcatgtaccccaaagtgtccatgggtgtaactgagcattgggatgaaactggcgatgcgggt

SEQ ID NO: 150 - protein, 200 aa, Physcomitrella patens
MGSLGRQGCLLVGVLFYLSMAIGAQAQSYPGLQAAFNSWTPKQIIPDKNGRKVQLVLNNSSSAYTGMGSKQSWLFGGIGAWIKLPANDSAGTVTTFYMSSTGPKHCEFDFEFLGNSSGQPYLLHTNIFVDGVGGREQQIRLWFDPTAAFHYYNFQWNNDVLVFFIDNTAIACSEPRRHRAIMYPKVSMGVTEHWDETGDA

SEQ ID NO: 151 - DNA, 1463 bp, Physcomitrella patens
  CDS (268)..(1128): c_pp004089354r
ggggcgtctaactagtggtcccccgggctgaggcaccggcacagcgatggtgcagcggattcaaccccacatcgatctggaactttgtcgttagtgctcacccagaagaagagcttctgcggagtagtcttcacccctatagtgccgacctagtggttatctgagcatttgatcgcaacgatttcacctagagcggtggagtgattttcagctgctgcctgataggaaggattgtctaacgggattgggggaacgtgcaatctagcaatggggtcgctcgggggttcgcgtagcaccctgctgattttgctgctactgtgtttgagcttggctgttggcggtcgcgcccaaacgcttgctcagcagttcactccgtggactgaaaatgcgaggttcactactgacactcaaatgcagctcaccttggatcaacgctatgcagctggggcaggatccgtgaacgtttggacgtacgtcgacatcagcgcgtacataaagatgccgccattcgattccgctggtactgtgacaacgttctacatgtcgtctcagggtgaccagcattacgagctggacatggagtttttgggaaacactagcggacagcccttcctgcttcacacgaatgtgttcgttgatggggttgggggtcgcgagcagcaaatgtacctgggattcgacccctctgctgacttccactactacagattccggtggagtaaggatatggttgttttctacgtcgataacaaacccgtccgagttttcaagaatctggaaggcacggtaccggggactaaatacctgaaccagcaagcaatgggggtgtacataagcatctgggacggtagcagttgggccacgcaaggagggcgtgtgcccatcaactgggcttccgctccattcactgcgacgtaccaggacttcgcactgaatgggtgcgtggtagaccccaacgatcccaatggagttgcagcatgccagaactctccgtatgcaaccggagcagccttgagcaatcaggaagtttatgagttggggcagaacaaagcttacatgatgaaatacgactactgcgacgacagggttcgatacccagatgtgccacctgaatgtccttacaacaacgtgttgaatacggaatgagtcgtgtacatgttacgtgctagctatttggggcgtggttgcctagtgaagatatagttgcgtagaggtcatctgattcttttgtatattaattgtacgcggaggtcgattctttgacgactatggacagatgtggcccgtcagctcagagtagtaaaagactaggaattttctttagtggaaagaataacgatctctttccacctgagttggtacattctatttgtaataatgctgatgctcatttggcagaagctatgaattctttgacttcaagtcatgtctctcaaaaaaaaaaaaaaaaaacctgcagcccg

SEQ ID NO: 152 - protein, 287 aa, Physcomitrella patens
MGSLGGSRSTLLILLLLCLSLAVGGRAQTLAQQFTPWTENARFTTDTQMQLTLDQRYAAGAGSVNVWTYVDISAYIKMPPFDSAGTVTTFYMSSQGDQHYELDMEFLGNTSGQPFLLHTNVFVDGVGGREQQMYLGFDPSADFHYYRFRWSKDMVVFYVDNKPVRVFKNLEGTVPGTKYLNQQAMGVYISIWDGSSWATQGGRVPINWASAPFTATYQDFALNGCVVDPNDPNGVAACQNSPYATGAALSNQEVYELGQNKAYMMKYDYCDDRVRYPDVPPECPYNN

SEQ ID NO: 153 - DNA, 1009 bp, Physcomitrella patens
  CDS (1)..(501): s_pp002010066r
ggcatcacgctggaggaatggtggcgaaatgagcaattctgggtgatcggtggcacgagcgctcacttagctgccgtctttcagggtttcctgaaagtcatcgccggggtcgacatctccttcacgcttacatccaaggcaactggggacgagggggatgacgagtttgccgatctgtacgtggtgaagtggagcgctctcatgatccctcccatcaccatcatgatcaccaacgtagtggctattgcggtgggcacctcgcgccagatttacagcaccatcccggagtggagcaagctcatcggcggcgtcttcttctccttgtgggtgctctctcatctctacccctttgccaagggcctcatgggccgcaagggcaaaactccgaccattatctacgtgtggtcaggtttgctctccgtcatcatctccctcatgtgggtgtatataaatccgccttcaggaacttctgtcactgggggcggcctctcctttccttgaaaattctttagcaaatttccaacagaaatttgcacagcatagctaccgctaacttgcccgacattggactagcatcggaccagctcactttcagcttatcacttccggtcccttgcacgcacacatgccttgaggattgcaaaactggactcggagagatccagtgcggctgtatcgaaaattgtgggaactgacccagagtaaggaattctggtgcacatcatttttctgctgtgagccgcaaagaaggatcttgagaaacgaaaaacaagcggagcaagcacgcggcgttgagaactgcaggtgccggtcataaccactgacgaatcatcgcccctcgcagtgtagattgctcattgcgaacggtgattaatgcgatcctcatgcatcgagttcggtaagatgaaccttaggcattcaagtccatgggcctagcgcgttgcatcttctgtagagttgatcatttgttcaataaaaaatttgacccagttttgaaacctcaaaaaaa

SEQ ID NO: 154 - protein, 166 aa, Physcomitrella patens
GITLEEWWRNEQFWVIGGTSAHLAAVFQGFLKVIAGVDISFTLTSKATGDEGDDEFADLYVVKWSALMIPPITIMITNVVAIAVGTSRQIYSTIPEWSKLIGGVFFSLWVLSHLYPFAKGLMGRKGKTPTIIYVWSGLLSVIISLMWVYINPPSGTSVTGGGLSFP

SEQ ID NO: 155 - DNA, 1337 bp, Physcomitrella patens
  CDS (187)..(1167): c_pp001002092f
cagccatcgaatgctcatcggctctgctcgattcctttagagcttaatcgcggatcggcggcggcagcagcagcagcaccgagtgcagcgagcccatccatctcgttgcaacgcaggaactggagcactccgagtcgtagcgatttcgagagcttcgttgcgcgcgagtgcttgtgttcgggagcaatggcattggctgcgtgcagggctgcgcactccgttgcgggggcttcgccgtcgtctctcgctgctgctgctgccaaaccctcttcgtcgctcgcgcgcccccaattcgctggactgcgccgtgctgatgtcgccaacgagtcatcgttcggggcagtcttgtctcaacggttgcagagtgcgggtacagggagcaggggagtcgtctccatggctggaactggaaagttcttcgtcgggggcaactggaagtgcaatggcacgactgagagcattaagaagcttgtggatgagctcaacagtgtcatgctcgaggagggtgtggaagttgtcgtgtcgccaccatatctgtacataagccaggtgctgggatcgttgtccaataggattgaggtcgcggctcagaactcgtgggttggaaagggtggagcgttcacgggggagattagtgcggagcagttggctgatgctggcgtgaaatgggtgattcaagggcattctgagcgaagacatgtgatcggcgagaccgatgccatgattgggaagaagagcgcgtatgcattgtcccaaggcctaggtgtgatcgcctgtgtgggggagaagcttgaggatcgtgaagcgaatcgcaccaccgatgttgtgttcgagcagttgcaagcttacgctgatgctgttggatcggactggtcgaacattgtggtagcgtacgagcccgtctgggctattggaactggcaaggtggccagccctcagcaagctcaagaggtgcacgctgccatccgtcagtggttgaaggagaaagtttctgatgatgtgtcttcaaagacccgcatcatctatggtggctcagtgaacggtgccaacagcgccgagcttgccacacaagaggacattgatggattccttgtcggaggagcttcactgaagggtgctgagtttggtgttatttgcaacgcggtcactgcaaagaaagttgctgcataagtcgattgctggctctgcttaattttgtctgttgtagccaaatcgcttgccttacacaacttgtttttgttttaggttagtgaacatgttgggccttgcaaatccgtgtatggggtatttttggtgaatgtccactgaggtgtctagtttgcgagctgagctaaaaa

SEQ ID NO: 156 - protein, 327 aa, Physcomitrella patens
MALAACRAAHSVAGASPSSLAAAAAKPSSSLARPQFAGLRRADVANESSFGAVLSQRLQSAGTGSRGVVSMAGTGKFFVGGNWKCNGTTESIKKLVDELNSVMLEEGVEVVVSPPYLYISQVLGSLSNRIEVAAQNSWVGKGGAFTGEISAEQLADAGVKWVIQGHSERRHVIGETDAMIGKKSAYALSQGLGVIACVGEKLEDREANRTTDVVFEQLQAYADAVGSDWSNIVVAYEPVWAIGTGKVASPQQAQEVHAAIRQWLKEKVSDDVSSKTRIIYGGSVNGANSAELATQEDIDGFLVGGASLKGAEFGVICNAVTAKKVAA

SEQ ID NO: 157 - DNA, 1060 bp, Physcomitrella patens
  CDS (3)..(917): s_pp013006066r
cagctatcgctgcttctttttctgctcccctcgcgtctgcccctgccttctccggcctccgtcgcctccctcttgctcccgcttcgtctcccgctttcggtgtcgtcttctctttgagtgaggggaaggggcacagaggtgtcgtcaccatgactggggccgggaagtttttcgttggcgggaactggaagtgcaatggcacaactgagtcgatcaagaagctcgtggaggatttgaacagtgcccaaattgaggacgacgttgatgtcgtcgtcgctcccccgtttttgtatatcagccaggtggtcgggtctttgacggaccgcattgaggtctccgctcagaactcttgggtcggcaagggaggagccttcactggtgagattagcgccgaccagctggtcgatgtcggtgtgaagtgggtaattcagggccactctgagcgccgacacgtcattggcgagtcgaattcaaccgttgggaagaagagcgcgtacgcattgtccaaaggcttgggattgattgcttgcgttggagagttgctcgaggagcgcgaagccggccgtaccacagatgttgtgtttgagcagctgcaagcatatgccgatgagatctcagattggtcgaaggtggtgattgcctacgaaccagtgtgggccattggaactggcaaagttgcctctcctcagcaagcgcaggaggtgcacagcgccattcgatcgtggttgagtgacaagatctcgccagaggtgtcctctgcgactcgcattatttacggtggttctgtgaacggagctaacagtgccgagcttgccaagcaagaagatattgatggtttccttgtcggtggagcctcattgaaaggacctgagttcgccacaatctgcaatgctgtcaccgcaaagaaagtttctgcataatctcgtacattgaatgcgctggtttagccgaatgacttagactaagcgcctccctttgtaaatcgctctcggacatgctgtaatggtagcccatgtttatggtaaaaaatcattgtaattcttcaaaaaaaaaaaaaaaa

SEQ ID NO: 158 - protein, 305 aa, Physcomitrella patens
AIAASFSAPLASAPAFSGLRRLPLAPASSPAFGVVFSLSEGKGHRGVVTMTGAGKFFVGGNWKCNGTTESIKKLVEDLNSAQIEDDVDVVVAPPFLYISQVVGSLTDRIEVSAQNSWVGKGGAFTGEISADQLVDVGVKWVIQGHSERRHVIGESNSTVGKKSAYALSKGLGLIACVGELLEEREAGRTTDVVFEQLQAYADEISDWSKVVIAYEPVWAIGTGKVASPQQAQEVHSAIRSWLSDKISPEVSSATRIIYGGSVNGANSAELAKQEDIDGFLVGGASLKGPEFATICNAVTAKKVSA

SEQ ID NO: 159 - DNA, 856 bp, Physcomitrella patens
  CDS (2)..(778): c_pp004048178r
gcaccaggcaggattgtctttcaatccatgtaatgtacagccatcgtcgcggcctgtatcgcagcccgttgtgacggtatctaactctgcatcgcaatcgtatgtgttctcttctagggggagatcaccatctcttccctcgcttaagtcatcatttcttcatcctatgactgctaatcgctccaatcgagcgatcaggaaaggtgtcacttcacctaggttgcattgcaccacttcccaagccagagatatggacgacttggtggtatgctttggagagctcctgatagattttgtgcccactgtgggcggtctatcgcttgctgaagctcccgcattcaagaaagcgcctggaggtgcacctgccaatgttgcttgtgggatagctaagcttggcggaaacgctgcttttgtcggaaaagttggcgacgatgagtttgggtacatgctttgcgaagttctcaaggataacaaagtgcaaaccaagggtgtcagattcgatgctcaagcaagaacagccctcgcttttgttacattgcgcgacgacggggagcgggaatttatgttttaccgcaatcctagcgccgacatgctcttccagacagatgagttggatattgagttgttgaaccaagcttccatcttgcattatggctccatcagtttgatcacagagccatctcgctcgacccacttggaggctatgcgcattgccaaagaggcaggcgcactcctggtcctacgatccaaaccttcggctgcccttgtggccatctgctgacgccgcaaaagaggggatatgtcgattgggatccgggtgatttctcaggtcggacgaggggtctcttctactgtg

SEQ ID NO: 160 - protein, 259 aa, Physcomitrella patens
HQAGLSFNPCNVQPSSRPVSQPVVTVSNSASQSYVFSSRGRSPSLPSLKSSFLHPMTANRSNRAIRKGVTSPRLHCTTSQARDMDDLVVCFGELLIDFVPTVGGLSLAEAPAFKKAPGGAPANVACGIAKLGGNAAFVGKVGDDEFGYMLCEVLKDNKVQTKGVRFDAQARTALAFVTLRDDGEREFMFYRNPSADMLFQTDELDIELLNQASILHYGSISLITEPSRSTHLEAMRIAKEAGALLVLRSKPSAALVAIC

SEQ ID NO: 161 - DNA, 979 bp, Physcomitrella patens
  CDS (1)..(798): c_pp001074086r
cggcgcgcaggacaggcagaggacgagacaaggggaaatgagtctagcagtgtgcaggatgatatagagaaggggtggtcttcggtgcagtgcttgccgaggcatatctggttggatgaagaatcgagtgcgaatttggtgcagtggccgattgaggaagtcgataagcttcggcggaatgaaatgacggagaagaatgtggaggttggggtgggtaaggttgtgcccgtcaaggcggcgaagggcgcgcagcttgacattgtggtagatttcgccctgcctgagaagagcgagggattggaacaaaacccaaacctgctggcggagatgggacatttgacatgcagcgatttggtacccaaggggtcgaatgcagctggaccacatagcttcggcccgtttggtgttcacgtgcttgcgaccggtgatctccaggagcggacgtccatcttcttccatttgatacacgatggcaagcaccagaactggaagacgctcttctgcggcgaccagagccaatcctccttgcagcaggacgtcgacaagacggtgtatgggtcctacgtgcgcgtggatgacagcgacaaggtgctgtccgtgcgcattctcgtcgaccactccatcgtggagagcttcgcccaaggcggccgcacggtaatgacatccagagtatacccggagctggcggtgaaagacgccgctcacgtgtttttgttcaacaacggtactgagcccgtgacagtgaaatcggtatccacctgggagatgaagagtgtcaacatcaagttttacaaaccttgatttgcacgctctttccgcttaactccggttgataactagccgaaatgttgctttcccaatttagaattgtttggacatcctcgaagctcaagctgctgcatgcatacagagatattcaaaatcatgtacacatctccacttgtaataaaacataataaaccactctgtttctcgtgcc

SEQ ID NO: 162 - protein, 266 aa, Physcomitrella patens
RRAGQAEDETRGNESSSVQDDIEKGWSSVQCLPRHIWLDEESSANLVQWPIEEVDKLRRNEMTEKNVEVGVGKVVPVKAAKGAQLDIVVDFALPEKSEGLEQNPNLLAEMGHLTCSDLVPKGSNAAGPHSFGPFGVHVLATGDLQERTSIFFHLIHDGKHQNWKTLFCGDQSQSSLQQDVDKTVYGSYVRVDDSDKVLSVRILVDHSIVESFAQGGRTVMTSRVYPELAVKDAAHVFLFNNGTEPVTVKSVSTWEMKSVNIKFYKP

SEQ ID NO: 163 - DNA, 1499 bp, Physcomitrella patens
  CDS (482)..(1390): c_pp004102322r
cggcaccagcgtgactccgtgtgtgagaagtatcgtttgttggagtgcgctgtcgatgccagtgtgtgttgggcgcgtgtcggaattaagctggtcgtgaaggtgctggcactccttgactgggtcgaccttggcgtgtgggaggagacttttgagatttgggagtttcgtttttgttgcgtgctgtgggttgttctgcacgagaggctttgcaggggagtgaattgggttcgctctgttagtcgtcctggctctcttttcgttcccgcgtgtggctagatcgagcgccgaagcactctgcaaggcgttctactgtcctgttgcttcgagattagagcccttgcaagcccggtcgttgttcgtgtctcccatcgcttcgcatcctaattcctcggtcgcttctgtatgtggtgcttccttctctctgagatcaagcaccgttttgcattttagaggctctagaaaaggcttggtttcttgcatggcaagcaattctattgccggagagctcatccctgagagtgttgagaagaagcaagtggagatgggcagcactgaagagaatttagatgcaacgcatggatttacgcgacctgaaatgatgaagaagaaaccgctggttggtagcgtcgacttgtatgatcgccacgtatttttacgctacaaccagccaagtagctggccagcgaaagtagaagctgcagactaccatcctttgccatctaaactggtcagtacgctgcgtagcaagagaaacgaattaccgaagaagactcgcttgactattgccgatggccaagatgagcctgaaaggacaaacggagatattttggtgtttcccgacatggtgaaggggaatttcaggaatcagatgttgaatctttgtgtcgatgaggtactgttgaagggggacaagtgggccttaggggaatctgagcctcttgttggaacccatgtgttcatctgtgcacatggcagtcgtgacaagaggtgtggtgtatgcggacctcctttaagagagcgtttcaatcaggaaattgctctgcgtgggctaggtgaacaagtgtttgtaactatttgctctcacattggaggccataagtatgccggtaacgtgattgtgtttagacctgatggaggttctggaggttgctcgggtcattggtacgggtacgtcactcctgatgatgtcccagagataatggagaagcacattggacttggcgaggtggtgggtcggctttggaggggtcagatgggattgactgaggatgagcagaaggaagttcagcagaaaaggaacccctccagtaatctaactcaggagggcactaaaccagaagggaaggtggatgcagcttcaacatctactggaggcaactgctcctattaggaaaaagcctcctcctcctctacttccacgcattgggtcacgcgacctgcaccaaatgtgtggggacgaacaggaagaagaaggtccacttaaaccgtacgaggag

SEQ ID NO: 164 - protein, 303 aa, Physcomitrella patens
MASNSIAGELIPESVEKKQVEMGSTEENLDATHGFTRPEMMKKKPLVGSVDLYDRHVFLRYNQPSSWPAKVEAADYHPLPSKLVSTLRSKRNELPKKTRLTIADGQDEPERTNGDILVFPDMVKGNFRNQMLNLCVDEVLLKGDKWALGESEPLVGTHVFICAHGSRDKRCGVCGPPLRERFNQEIALRGLGEQVFVTICSHIGGHKYAGNVIVFRPDGGSGGCSGHWYGYVTPDDVPEIMEKHIGLGEVVGRLWRGQMGLTEDEQKEVQQKRNPSSNLTQEGTKPEGKVDAASTSTGGNCSY

SEQ ID NO: 165 - DNA, 1777 bp, Physcomitrella patens
  CDS (135)..(1559): c_pp004089380r
gctaaacctagtgatgcacgagccgttttgcccatcggcacgaggaaacctcgatcatctccgcctgtgcagcagctgcagccgttctgttggagtgcttgtgtggtgccgcgcgcggtctttaagcttgaaggatggagtccctcgcgctgcgatctgccgtggttgccaccggattgacctccagtgtggcatcccaaacctctgtgcagacccgcgctacggtttcgtctgctttcatcgggaagagcatacgtgtgaacactaaactcaacgcctcagctgtgcccgtccagcagaagttccgctacgtgcgtgccgacgctggtgcacagactgcgcaggttgagaccgttgagaagaaggctagcattaaggacgttcccgagtcggaattccagggcaaggttgtgttcgtgcgtgctgatctgaacgtacctctcaatgatgcatgcgaaatcaccgatgacacccgaatccgtgcctccctcccgaccatccagcatcttacgaaggccggagccaaggtggtgctcgctagccatttgggtcgccccaagaagggccctgaggacaaattcagcttgaagcccgttgcagggagactgaccgagttgcttggccagaccgtggaattggcccctgattgcattggtgctgaagtagaatctaagattgcagcactgaagaatggtgaagttctccttctggagaatgttaggttctacaaggaagaggagaagaacgacagcgagttctcccaaaagcttgccaagggtgtcgatatcttcgttaacgacgccttcggcactgcccaccgcgctcactcatctactgcaggaattgctgagtacgttggcaagactgtggctgggttcctcttggagaaggagttggcttatctcgctggcgccgtgaaggccccagccaggcctttcgttgccattgttggaggcagcaaggtctcatcgaagattactgtgattgagtccttgatgaacgtgtgcgacaaggtcattttgggaggtggcatgatctttaccttcttcaaggcagacggcaaggatgttggaagttccctagtggaggatgacaagattgatttggccaaggacctcgtggctctagctaagaagaagggcgttgagctcattctccccgtcgatgtcaccgcagctgataagttctctcccgaagccaacactcaagtgtgcagctcctcgaacatccctgccggctggatgggactagacattggcccgaaggcaatcgaccaattccaggatgccctgaagggcgccaagacggttctgtggaacggaccgatgggagtgttcgagttcgagaagttcgcggacggaacaactgccgtcgctaaaactttggcaggtttgaccaaggagggtgccatcaccatcattggaggaggtgactccgtcgcagccgttgagaaggctggactcgccgaccagatgagccatgtgtccaccggaggaggggccagtctcgagttgttggaaggcaaggtattgccaggagttgctgctcttgacaacgcttaaatgcctcttttgccgaagaggtgaattccgtccacgaactcgcagtagagtgacaatgtcactgagtgcacccccggctctggctgtctaatcatatgattcatttttttggtagttttttttgtgatttgcctttgtaccagtaaacacagattatgaccagtaaagaactccagctcgatttacctggatgcttggtattctcttaaaaaaaa

SEQ ID NO: 166 - protein, 475 aa, Physcomitrella patens
MESLALRSAVVATGLTSSVASQTSVQTRATVSSAFIGKSIRVNTKLNASAVPVQQKFRYVRADAGAQTAQVETVEKKASIKDVPESEFQGKVVFVRADLNVPLNDACEITDDTRIRASLPTIQHLTKAGAKVVLASHLGRPKKGPEDKFSLKPVAGRLTELLGQTVELAPDCIGAEVESKIAALKNGEVLLLENVRFYKEEEKNDSEFSQKLAKGVDIFVNDAFGTAHRAHSSTAGIAEYVGKTVAGFLLEKELAYLAGAVKAPARPFVAIVGGSKVSSKITVIESLMNVCDKVILGGGMIFTFFKADGKDVGSSLVEDDKIDLAKDLVALAKKKGVELILPVDVTAADKFSPEANTQVCSSSNIPAGWMGLDIGPKAIDQFQDALKGAKTVLWNGPMGVFEFEKFADGTTAVAKTLAGLTKEGAITIIGGGDSVAAVEKAGLADQMSHVSTGGGASLELLEGKVLPGVAALDNA

SEQ ID NO: 167 - DNA, 1566 bp, Physcomitrella patens
  CDS (81)..(1160): c_pp004044298r
gttttctgagaccttgtagcggagctggtgagtaataaaccggccagccctgactgtcgggattgacccacagtctcgcaatggcgaagggcgggtcgagcgccactgccaacagcggcgtgttacagagaattgtcttgagctacacgtatgtcgcagtatggatctttctcagcttctccgtgatcatctttaacaaatatattcttgaccgcggaatgtacaactggccatacccagtctctctgactatgattcacatggcgttctcctccgggctcgccttcctcctcgtgcgcgggctgaagttggttgagccctgcgccgcgatgacgaaggacctctacttcaggtccatcgtccccatcggcctcctcttctcgctctctctgtggttctcgaattcggcttacatctaccttagcgtctccttcatccagatgctcaaggcgctcatgccggtggcagtctactctcttggggtacttttcaagaaggatgtattcaactcttcgaccatggctaacatggtcatgatctccattggtgtcgccattgcggcctacggggaggcgcggttcaatgtctggggtgtcacgctgcagcttgcggctgtatgcgtggaagccctccgtcttgtcttgatccaaattcttctcaactcccggggaatttccctcaatcccattacaacactctattacgtcgcgcccgcgtgttttgtcttcctctctgtcccttggtatctcatcgaatggccgaagctgctggtaatgtcgtccttccacttcgacttcttcacgttcggcctcaactctatggtcgcgttcctgctcaacatcgccgtctttgttctggtcgggaaaacatccgccctcaccatgaatgtggcgggcgtggtgaaggactggctcctcatcgccttctcctggtccgtcatcttggaccgagtgactttcatcaatctcttcggctacggcatcgctttcgtcgccgtctgttactacaattacgccaaactgcagaccatgaaggccaaggaacagcagaaatcacagaaggtcagcgaggacgaggagaatttgcggctgctggattctaagctggagagactcgatgagagttcatctccttctcacaagtccgacgctcaaacccactaatttttttcttttatttttcaccttttttcttcccaccacttcaagccgctgaagcccattcaaccccatgagaaagtcccatcagatgtcttctcccatttctgtggcatgatcacttggaagtggatgtgttattgcaaaaggcagcgattttcacatccgacactcaaaccctagctgtcgttacatggatgcgagctttaggacaggcaggcagatgcagatcccggaggtgtgagtcttgggtacagttgtagcggcactgggtgtcggtgagagatctttggtaggattaagaagttggctgcggaggactctcactccctccctcgctcaattttctattatgtctccgttacccattttttaatttttaacataagttttgcagcgtttaaaaaaa

SEQ ID NO: 168 - protein, 360 aa, Physcomitrella patens
MAKGGSSATANSGVLQRIVLSYTYVAVWIFLSFSVIIFNKYILDRGMYNWPYPVSLTMIHMAFSSGLAFLLVRGLKLVEPCAAMTKDLYFRSIVPIGLLFSLSLWFSNSAYIYLSVSFIQMLKALMPVAVYSLGVLFKKDVFNSSTMANMVMISIGVAIAAYGEARFNVWGVTLQLAAVCVEALRLVLIQILLNSRGISLNPITTLYYVAPACFVFLSVPWYLIEWPKLLVMSSFHFDFFTFGLNSMVAFLLNIAVFVLVGKTSALTMNVAGVVKDWLLIAFSWSVILDRVTFINLFGYGIAFVAVCYYNYAKLQTMKAKEQQKSQKVSEDEENLRLLDSKLERLDESSSPSHKSDAQTH

SEQ ID NO: 169 - DNA, 1536 bp, Physcomitrella patens
  CDS (82)..(1239): c_pp004075307r
gcagcaggcaccaccttcatcgccgccgcgctccgtcttgccctgtgtgtcagggttcccccggactgagcatctacgacaatggctgaccagcgttgccccagcgtagtgagcaagatgggagggacgtcatacctgggatccaggttggcacctggccgcgccatgtaccccgcgtcggagatgagcactccgttcgcggccgctgccaagctgggtgcgatgcccaggcagacggggctgagctccctgtgccccatcgacgtgaccggcgggaggaacatgtcgagccaggtgttcgttccggccgcgaacgagaagacgttcgcgtcgttcatgaccgactttctgatgggcggtgtgtcggccgcggtgtcgaagacggccgctgcgcccatcgagcgcgtgaagctgctgatccagaaccaggacgagatgctgaagtcggggcgtctgtcgcacccgtacaagggcatcggcgagtgcttcagccgaacggtgaaggacgagggaatgatgtcgttgtggcgtggaaacacggcgaatgtgatcagatactttccgacgcaggcactgaacttcgcgttcaaggactacttcaagtcgctgttcgggtacaagaaggacaaggacgggtattggaagtggttcgcgggtaacttggcgtcgggaggtgctgcgggagcgtcgtctctgctgttcgtgtactctctggactacgcgcgtacccgattggcgaacgacgcgaagtcgtcgaagaagggaggaggcgagaggcagttcaacgggctggtggacgtgtacaagaagacgttggcgacggacggaatcgcggggctgtacagagggttcgcgatctcttgcgcgggtatcatcgtgtacaggggtctgtacttcggaatttacgactcgctgaagccggtggtgttggtgggcaacctggagggcaatttcttggcgagtttcttgttgggatggggaatcacgatcggagcgggtctggcgtcgtaccccatcgacacggttcggcgtaggatgatgatgacctccggagaggcagtgaagtacaacgggtcgatggacgcgttcaagcagattttggcgaaggagggagcgaagtcgttgttcaagggcgctggtgcgaacatccttcgtgcggtggctggagccggagtgttgtcgggatacgatcagttgcagatcttgcttctgggcaaggcctactctggaggcagcggctgagtgcttcgtagcggattatgaagagaattttgttgccctggtcaatctttaatttagcactttcttttttgtagtgtaactttttgagttctttcgcgttctgactatcatagtgcacatgcgtatagtgcgctgagctggttatcgtgttagttttgtgtcttcgaattaacttcgagattactcaacttaggcgccataatgtgcttatttacaactcttatgaagcaagaattgctggcttctggttcgtccattgtgctctctttttatcttcatttaatatcattctat

SEQ ID NO: 170 - protein, 386 aa, Physcomitrella patens
MADQRCPSVVSKMGGTSYLGSRLAPGRAMYPASEMSTPFAAAAKLGAMPRQTGLSSLCPIDVTGGRNMSSQVFVPAANEKTFASFMTDFLMGGVSAAVSKTAAAPIERVKLLIQNQDEMLKSGRLSHPYKGIGECFSRTVKDEGMMSLWRGNTANVIRYFPTQALNFAFKDYFKSLFGYKKDKDGYWKWFAGNLASGGAAGASSLLFVYSLDYARTRLANDAKSSKKGGGERQFNGLVDVYKKTLATDGIAGLYRGFAISCAGIIVYRGLYFGIYDSLKPVVLVGNLEGNFLASFLLGWGITIGAGLASYPIDTVRRRMMMTSGEAVKYNGSMDAFKQILAKEGAKSLFKGAGANILRAVAGAGVLSGYDQLQILLLGKAYSGGSG

SEQ ID NO: 171 - DNA, 1905 bp, Physcomitrella patens
  CDS (1)..(1440): s_pp001024093f
cgcattcagaagcgggctacatcttccgtgcgcgcccaagctgctgatggagaagcctcgggggatgttgccactagacaatctaatcctgctaccactggaatggtcttgcctgcagttggtattgcctgccttggggcaatcttgtttggttaccatctcggggtggttaatggtgcattggagtacatttctaaggatctagggtttgccacggatgctgtaaaacaaggatgggtggtaagctcaactctagctggtgccactgtgggttcctttactggaggcgcccttgctgacaacttaggtcgcaagcgtacattccagattaacgccgtgcctcttattgtgggcactcttctcagtgcaaaagcaaccagtttcgaggctatggtgattggaagaattttggttggtgttgggattggagtttcatctggtgttgtgcctctatacatttcggaggtctcgcccacagagattcgaggtaccatggggacattgaatcagctctttatttgcgtgggtatcctgttagctctgattgctggccttcctttgggcagtaaccctgtctggtggcgcaccatgtttgccttagctacagttcctgccgttttgctgggtttaggcatggcgtactgtccggagagtccacgctggctatacaagaatggtaagaccgcagaggcggaaaccgcagtaaggagactttggggcaaggcaaaggtcgagagttcaatggcagatttgaaggctagcagcgtggaaacagtgaaaggtgacactcaagatgcaagttggggcgagctatttggcaaaagataccgtaaagttgtcacggttggaatggcgctcttccttttccaacaatttgccggaatcaatgctgtggtatacttctctactcaggttttcaggagtgctggcatcacgaatgatgtagctgccagtgctcttgtaggtgctgcaaatgtggcaggtaccactgtggcgtccggcatgatggataagcaagggcgtaagagcctgctaatgggcagcttcgctggcatgtcactttcaatgcttgtgctttcgttggcgctttcatggagcccccttgcaccgtactctggcacgctggctgtccttggaacagtttcatacattttgtccttctcccttggtgctggaccagtgcctggccttctgttgcccgagatcttcggtgctcgcatccgtgctaaggccgttgctctttctctcggtgtccactggatttgtaacttcatgattggactatttttcttgaacgtcgttcagaagttcggtgttagcacagtatatctcttcttctctgcagtatgcgcggcagcaattgcctatgtaggcggtaatgtggtagaaacaaaggggcggtcactggaggacatcgaacgcgagcttagccctgctgtatagtagcatcagtcgaaaccatcacttttttctgaaagtgcaatgcagtggctgttggtgtggaaaagtctctcccgaacgggtgtctgacatgagctgcatgaatcgttgacgactcggtgtctagagtcggtctgcgccttctacgcaacgctgaagtgaattgaagagaattactgattgagtagtgtgtattatcctcgaaggccaggagtgttttgcgcaaccttcggtgtcactgttgaggacgatgctgacactcaacatggttaggtttgagcattatggttgtaattaggtgacacattagacattagggtgatggatacggtgttgcagttagagtgtcagaagttcgattcaggagcaggctttcgtgcatgtttggttacttaaatggttgtgtaaattactgcaaatcgctacgttgccggatatttctttgaagtaaacggccgctttttt

SEQ ID NO: 172 - protein, 480 aa, Physcomitrella patens
RIQKRATSSVRAQAADGEASGDVATRQSNPATTGMVLPAVGIACLGAILFGYHLGVVNGALEYISKDLGFATDAVKQGWVVSSTLAGATVGSFTGGALADNLGRKRTFQINAVPLIVGTLLSAKATSFEAMVIGRILVGVGIGVSSGVVPLYISEVSPTEIRGTMGTLNQLFICVGILLALIAGLPLGSNPVWWRTMFALATVPAVLLGLGMAYCPESPRWLYKNGKTAEAETAVRRLWGKAKVESSMADLKASSVETVKGDTQDASWGELFGKRYRKVVTVGMALFLFQQFAGINAVVYFSTQVFRSAGITNDVAASALVGAANVAGTTVASGMMDKQGRKSLLMGSFAGMSLSMLVLSLALSWSPLAPYSGTLAVLGTVSYILSFSLGAGPVPGLLLPEIFGARIRAKAVALSLGVHWICNFMIGLFFLNVVQKFGVSTVYLFFSAVCAAAIAYVGGNVVETKGRSLEDIERELSPAV

SEQ ID NO: 173 - DNA, 1668 bp, Physcomitrella patens
  CDS (1)..(1290): c_pp010010057r
atgctgctaagccccagctcaagcgctctcggagccagcacgagcaaagcccgaggtggcaacatcaaggcagatcctctcagagtaatcatgtttcaggggttcaactgggagtcgtggaagagctcgtgctggtatgatgtcatgggcgagactgcggaagatctagcagcagcaggcattactgatgtctggtttcctccttctagccattccgtgtccccccagggatacatgccaggaaggctttacgatttgaacgactgtaaatatggcaatgaagagaagctgagggaaaccattgaaaagtttcacagagtgggagttcggtgcattgctgatattgtcgtgaatcatagatgcggtgaagaacaagacgagaggggtgaatgggttatttttgaaggaggaacgcccgatgatgctctcgactggggtccttgggctatagtcggagatgactatccctatggtaacggaacaggtgctcccgacaccggagatgactttgaggctgcacccgacattgatcacacaaacgatatcgttcaaagcgaccttatcgtctggatgaattggatgaagttcaaaattgggtttgatgggtggagattcgactttgccaagggttacggtggatacttcgtcggtcgctacatcagaaaaactgaaccacagtttgcagttggggagttctggacgagcttgaattacggacatgacggtctggaatacaaccaggatagtcacaggcagcaactggttgattggatccacgcaacgaaggagaggtccactgcattcgatttcaccaccaagggtatcttgcaagaggccgtgaaaggtcagctgtggcggctgcgggatccgaacagcaagccaccagggttgatcggttattggccttcaaaagcagtaacgtttctcgacaaccatgacacaggatctacacaaggacactggccttttcctggtgaacatatcatgcaaggctatgcttacatactcactcatcctggcaacccttgtatcttctacgaccatttctatgattggggtttgaaagaggagatcaaacgcttattgatcgtgcgcaagcggaatgacatcaatgcgaagagcaaagtgcacatttgttgcgctgagcacgatttgtacgttgcgaagatagatgatcgtgtcatcctcaagatggggccccgatatgacataggcgatctagctccaactgcgacgaatataaaattgcagctgtggggaaaagactactgcgtatgggagaagtgtacataatttcttacagagggttgcatttatcttcaagcggggcctagccagccgacgcgtgtggaatgtagtttcctaatccgtgtagctattttttcagtatcaatgtattgtttttaattttggagtgaagctggtaacagatttgagacgtttgatgacgtatgtcacgtttactacctgcttcagaagcgagcgtgagacacttcatcaatcttctgccaatatcaatcttcaggaacaattcgtgtacaggagaagtaacttatttagagtagaatagtgcgccgttactattcctcagccatgttctgaggtaccacttatgttacaactcgtttaaaatatatatatcgctctattggagtgtaacatagatgt

SEQ ID NO: 174 - protein, 430 aa, Physcomitrella patens
MLLSPSSSALGASTSKARGGNIKADPLRVIMFQGFNWESWKSSCWYDVMGETAEDLAAAGITDVWFPPSSHSVSPQGYMPGRLYDLNDCKYGNEEKLRETIEKFHRVGVRCIADIVVNHRCGEEQDERGEWVIFEGGTPDDALDWGPWAIVGDDYPYGNGTGAPDTGDDFEAAPDIDHTNDIVQSDLIVWMNWMKFKIGFDGWRFDFAKGYGGYFVGRYIRKTEPQFAVGEFWTSLNYGHDGLEYNQDSHRQQLVDWIHATKERSTAFDFTTKGILQEAVKGQLWRLRDPNSKPPGLIGYWPSKAVTFLDNHDTGSTQGHWPFPGEHIMQGYAYILTHPGNPCIFYDHFYDWGLKEEIKRLLIVRKRNDINAKSKVHICCAEHDLYVAKIDDRVILKMGPRYDIGDLAPTATNIKLQLWGKDYCVWEKCT

SEQ ID NO: 175 - DNA, 2068 bp, Physcomitrella patens
  CDS (168)..(1628): c_pp004072377r
cggcacccaaaggctcggaggaatggtggaggaagcactcaagtactcctggagactctatgtccgagcccaaagttggccatctttctcaaatgagtactatgtttccaaagcacaaatctcttcttgattgggagaatgcgggggaggagtggagtgaatatttgatgcacgagacggcaacaagcagaggtgttcgtggcggcgtccctgtgttcgtgatgctccctttggacaccgtaagcatgaataacactctgaacagacgtcgcgccttggacgcatctttgctggctctgaaatcggctggtgtggagggggttatgatggatgtttggtggggaatcgtcgagaaagatggccctcagcagtacaattggtctgcgtatcaagagttaattgatatggtgcggaagcatggtttgaaggttcaggctgtgatgtcctttcaccagtgtggtggcaacgttggcgacagttgcaatattcctctgcctccatgggtgttggaagaggtacgaaagaatccagacttggcctacaccgataaggctggaaggcgcaactcagaatacatctctcttggcgctgacaacgtgcccgctttgaagggaaggacaccggttcaatgctatgcggatttcatgaggagcttcagagacaacttcgacgattttttgggagattttattgtcgaaatccaatgcggaatgggacccgctggtgaacttcgttacccttcataccctgagagtgagggtaggtggcgttttccaggcattggtgagtttcagtcttacgacaaatacatgattgcgagcttgaaagccaatgctcagaaggttggaaagcctgcatggggttttagcggtcctcacgatgctggcagttacaaccagtggcccgaggaagcaggattcttcaagaaagatggcacgtggtcttcagaatatgggcagtttttcttggaatggtattcagagatgcttctggcccatggtgaacgcattttgtcacaagctactggcattttcaggggcactggagctatcatttcaggcaaagttgctggtatccattggcactacggcaccagaagtcatgctgctgagttgacggcgtggatactacaacactcggaccagggatggaatattcggccattgcccagatgttcgccaagtatgggggttaccctgaatttcacatgcatcgagatgcgagacttcgaacaaccatcacatgcactgtgcagccccagaaggtctggtgagacagcgtggcattgggcaacacgaaaggcacggaatttccaaatggcctggggaagaaacgctcttcccaggttcgacaattcagcgcatgaaccagatagttcgcgagtcgagattgcaaatgaacgagaaaggggattgtcaggaggatatgagccgatgtctgctttcaccttcttaagaatgtgtgagagcttgttccacagtgaaaactggagactgttcgttccgtttgtgcgccacatggaggaaggacgaacgtttcagccctgggaggaagaatcacacagaacgcagaatcatatgcatgtgactcagcccttgggccaagaagcagcctcgttgatgtaccactgatccgtaagcgtgtcatggattttagaggttggtgattgcctcccattttgcttgttacatacccatatacatatttgtagccagtacgatataccatacaaacatatacagacaaatacatgtattctcgaactagaggaaatcgccttccattacaggcatgaaaactttttgttctactcaacaatgagagtagatcaacagcgtaaaattatttcatagtctgactctctgtttcagtagatttggaaatactgaacaagggcaacagtctggcgtacggttgtattattatctaggacactagttactactccagtcagattgcaaccttagaatcaccactgcgtatcactggattttgtggtagaaaagaagtgggcagctgcttctgcagtcgttttgtgctagtatcgaatagggtgtaacacgaaaaa

SEQ ID NO: 176 - protein, 487 aa, Physcomitrella patens
MHETATSRGVRGGVPVFVMLPLDTVSMNNTLNRRRALDASLLALKSAGVEGVMMDVWWGIVEKDGPQQYNWSAYQELIDMVRKHGLKVQAVMSFHQCGGNVGDSCNIPLPPWVLEEVRKNPDLAYTDKAGRRNSEYISLGADNVPALKGRTPVQCYADFMRSFRDNFDDFLGDFIVEIQCGMGPAGELRYPSYPESEGRWRFPGIGEFQSYDKYMIASLKANAQKVGKPAWGFSGPHDAGSYNQWPEEAGFFKKDGTWSSEYGQFFLEWYSEMLLAHGERILSQATGIFRGTGAIISGKVAGIHWHYGTRSHAAELTAWILQHSDQGWNIRPLPRCSPSMGVTLNFTCIEMRDFEQPSHALCSPRRSGETAWHWATRKARNFQMAWGRNALPRFDNSAHEPDSSRVEIANERERGLSGGYEPMSAFTFLRMCESLFHSENWRLFVPFVRHMEEGRTFQPWEEESHRTQNHMHVTQPLGQEAASLMYH

SEQ ID NO: 177 - DNA, 2450 bp, Physcomitrella patens
  CDS (736)..(2055): c_pp001109095r
gcgcgccaggacaggcagaggacgagacaagggggaccgaggtccagagatcagctcgcaggacctcgaaactgagctctagtagcgtagaattagcgcaagggtggtgtaggtgaacagagcgacgagaccattctcgtcatcgtagttataagacgcatacgagattccgcagtgtcgagagtcgggtaccgagcccgcgcgagagtgaggatttgctacaggccttccttttatttgtcgacattattgtccgagtaggttgtggctgaagttgcggtggaacaggttgtgtgtagaactcgaagcagtcaagtttgcctgtcgaatttctcaaacttactacagttcttctcaaacaagaagtttgttagccgcgatggcagcaacagggactttcgcctccacccgttacaccgcactggcgaggccagctcgctgcgaagcagtcggctacgatgccaccatgcggtcgggctttaagggggacctcaagcctgcatcttctgggttcttggctggtggcggtcgtctggctctcgtcaccagcgttggtcccaaacgcacagttgccagagccaagaccatcggccttgaagtctccgctgtgttggccgaacgcccaatgggggctctgaccagacaagtaacacgggagatggagaaagaaatggagagggaaagggagaaggagaaatcgagggagagcatgagctcgaaggagcaggtgactacatgacgaaggtgttctcaatcatcctgggaggaggggcaggcactcgactccaacccctcactcttcgccgagcaaagccagcggttccacttgggggtggctatcgattgatcgatgtgcccatgagcaactgcattaacagcgggattaacaagatttatgttctcactcagttcaattctacgtcttcgatcaaccgccaccttgccaacacttacaatttcggcaacggttgcaacttcggtgatggttacgtggaggtcctggctgctgcccagaggcctggcttcggcggtgacaggtggttcgaaggtactgcggacgcagtgaggcagtacatgtggttgcacttggaagatgccaaaaacaaagacgtcgaagatgtggtgatcctgtccggggatcacctgtaccgcatggattaccgagatttcgtccagaaacacaaggattccggagctgatgtgactgtttcttgcatacccatggacgatagtcgtgcttctgattttggcttgatgaagatcgacggaaaggggcgaatcaaccacttttctgaaaagccgaaaggaaaggacctgcagtcgatgcaagtggacaccactgtactcggcctgtccgcggaggaggctcaaaagaagccatacatcgcttccatgggcatttacgtcttcaagaagagcgtgctcgccaagctgctcaggtggaggtatcccctcgccaacgactttggctccgagatcatccctaaggctgccaaggagttcaatgtgaacgcctacctctttaacgattactgggaagacattggaaccatcaaatccttcttcgacgcgaacttggcgctccgcagccagagaatccccaacttcagtttctacgacgctgaaaagcccatttacacatccgcccgctacttgcccccaaccaagattgagaagtgtagagtgaaggattcaattgtctcccacggatgcttcttgcgggaatgcagtgtagaggattccgtcattggaatccgatcccggctcgaggctgggtgcgatgtcaagcgcgccatggtgatgggagctgactcctatgaaaccgatcccgaagcggctgctttgttggcggaaggaaaggtacctttgggcgtcggcgagaactcaaagctgaggaattgtattgtggacaagaatgcaagaattggcaaggatgttgtcattgcgaacactgacaatgtcttggaagcggagagacaaagtgaaggtttttacatccgttccggaattgtagtagtatacaagaacgcggttatcaagcacggaactgtaatctaaattacgaatctttctccatatcgtgaaaactgcttccttgcaacgccggtccctggtgagctgatccttggacctcatcatcgacggtgaaaaagatatcagtacttatccgagttgtgagattctcagctcgtgattactgatcctcttcctcttgccaccctgaagattgccgcgcaggatctggtttgtgtcggtatatcagatcagtagttcttacataagactgaattgaaatgtacaaagagatcagatcatcgggagtggaaccgttcagaggaagaaacctaccgtatagtcaagtggacgaagattatggtatcaattttggagtgtaaagtgtgagcgacttctactgtccttgtatcataccccattgaagtaaagaattg

SEQ ID NO: 178 - protein, 440 aa, Physcomitrella patens
MTKVFSIILGGGAGTRLQPLTLRRAKPAVPLGGGYRLIDVPMSNCINSGINKIYVLTQFNSTSSINRHLANTYNFGNGCNFGDGYVEVLAAAQRPGFGGDRWFEGTADAVRQYMWLHLEDAKNKDVEDVVILSGDHLYRMDYRDFVQKHKDSGADVTVSCIPMDDSRASDFGLMKIDGKGRINHFSEKPKGKDLQSMQVDTTVLGLSAEEAQKKPYIASMGIYVFKKSVLAKLLRWRYPLANDFGSEIIPKAAKEFNVNAYLFNDYWEDIGTIKSFFDANLALRSQRIPNFSFYDAEKPIYTSARYLPPTKIEKCRVKDSIVSHGCFLRECSVEDSVIGIRSRLEAGCDVKRAMVMGADSYETDPEAAALLAEGKVPLGVGENSKLRNCIVDKNARIGKDVVIANTDNVLEAERQSEGFYIRSGIVVVYKNAVIKHGTVI

SEQ ID NO: 179 - DNA, 18 bp, Artificial Sequence
  Artificial sequence is a sequencing primer
caggaaacagctatgacc

SEQ ID NO: 180 - DNA, 19 bp, Artificial Sequence
  Artificial sequence is a sequencing primer
ctaaagggaacaaaagctg

SEQ ID NO: 181 - DNA, 18 bp, Artificial Sequence
  Artificial sequence is a sequencing primer
tgtaaaacgacggccagt